(19) United States
(12) Patent Application Publication
(10) Pub. No.: US 2023/0113948 A1
(43) Pub. Date: Apr. 13, 2023

(54) SYNTHETIC PROCESSES AND INTERMEDIATES

(71) Applicant: ARBUTUS BIOPHARMA CORPORATION, Warminster, PA (US)

(72) Inventors: Mahesh PALLERLA, Chalfont, PA (US); Ganapati Reddy PAMULAPATI, Warrington, PA (US); Jan SPINK, Hatboro, PA (US)

(73) Assignee: ARBUTUS BIOPHARMA CORPORATION, Warminster, PA (US)

(21) Appl. No.: 17/787,089

(22) PCT Filed: Dec. 17, 2020
(86) PCT No.: PCT/US2020/065666

§ 371 Date: Jun. 17, 2022

Related U.S. Application Data
(60) Provisional application No. 62/951,836, filed on Dec. 20, 2019.

(51) Int. Cl. A61K 47/54; C07D 491/048; C07D 207/08; C07C 213/08; C07H 1/00; C07H 15/08; C07C 231/12; C07C 231/02; C07D 207/46; C07C 201/12; C07C 227/04; C07C 231/10; C07C 237/06; C07C 237/16; C12N 15/113; A61K 45/06

(52) U.S. Cl. CPC A61K 47/549; C07D 491/048; C07D 207/08; C07C 213/08; C07H 1/00; C07H 15/08; C07C 231/12; C07C 231/02; C07D 207/46; C07C 201/12; C07C 227/04; C07C 231/10; C07C 237/06; C07C 237/16; C12N 15/1131; A61K 45/06; C07B 2200/13; C12N 2310/14; C12N 2320/32; C12N 2310/351

(57) ABSTRACT

The invention provides synthetic processes and synthetic intermediate compounds that can be used to prepare therapeutic conjugates. The invention also provides methods for treating HBV and/or HDV infection in a human by administering a therapeutic conjugate prepared by the synthetic methods of the invention.

### CROSS-REFERENCE TO RELATED APPLICATION(S)

This patent application claims the benefit of priority of U.S. application Ser. No. 62/951,836, filed Dec. 20, 2019, which application is herein incorporated by reference.

### BACKGROUND

Hepatitis B virus (HBV) is a member of the Hepadnavirus family. Infection of humans with HBV can cause an infectious inflammatory illness of the liver. Infected individuals may not exhibit symptoms for many years. It is estimated that about a third of the world population has been infected at one point in their lives, including 350 million who are chronic carriers.

Hepatitis D virus (HDV) is a small circular enveloped RNA virus that can propagate only in the presence of the hepatitis B virus (HBV). In particular, HDV requires the HBV surface antigen protein to propagate itself. Infection with both HBV and HDV results in more severe complications compared to infection with HBV alone. In combination with hepatitis B virus, hepatitis D has the highest mortality rate of all the hepatitis infections.

International Patent Application Publication Number WO 2018/191278 describes conjugates that are useful to target siRNA to the liver that are suitable for treating, e.g., HBV and/or HDV. Currently there is a need for synthetic processes and synthetic intermediates that can be used to prepare such conjugates.

### SUMMARY

In one aspect the invention provides synthetic processes and synthetic intermediate compounds that can be used to prepare therapeutic conjugates.

The invention also provides a method for treating HBV and/or HDV infection in a human by administering a therapeutic conjugate prepared by a method of the invention.

The invention also provides a method for treating HBV and/or HDV infection in a human subject comprising administering to the human subject, a therapeutically effective amount of a therapeutic conjugate prepared by a methods of the invention, and a second therapeutic agent that is useful for treating HBV and/or HDV.

The invention also provides a compound prepared by a method of the invention.

The invention also provides a therapeutic conjugate prepared by a method of the invention for use in medical therapy.

The invention also provides a therapeutic conjugate prepared by a method of the invention for the prophylactic or therapeutic treatment of HBV and/or HDV, optionally in combination with another therapeutic agent.

The invention also provides the use of a therapeutic conjugate prepared by a method of the invention to prepare a medicament for the treatment of HBV and/or HDV, optionally in combination with another therapeutic agent.

### DETAILED DESCRIPTION OF THE INVENTION

The following definitions are used, unless otherwise described.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethyl silyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

### Capsid Inhibitors

As described herein the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA. Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

The term capsid inhibitor includes compounds described in International Patent Applications Publication Numbers WO2013006394, WO2014106019, and WO2014089296, including the following compounds:

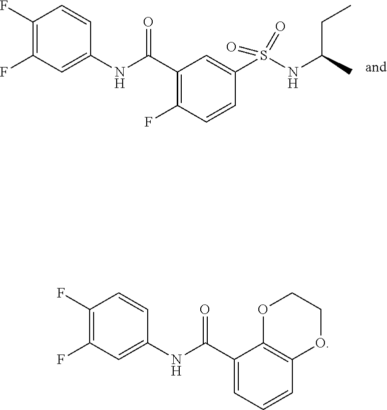 [SMILES: CC[C@@H](C)NS(=O)(=O)c1ccc(F)c(C(=O)Nc2ccc(F)c(F)c2)c1 ; O=C(Nc1ccc(F)c(F)c1)c1cccc2c1OCCO2]

The term capsid inhibitor also includes the compounds Bay-41-4109 (see International Patent Application Publication Number WO/2013/144129), AT (see International Patent Application Publication Number WO/1998/33501; and King, R W, et al., Antimicrob Agents Chemother., 1998, 42, 12, 3179-3186), DVR-01 and DVR-23 (see International Patent Application Publication Number WO 2013/006394; and Campagna, M R, et al., J. of Virology, 2013, 87, 12, 6931, and pharmaceutically acceptable salts thereof:

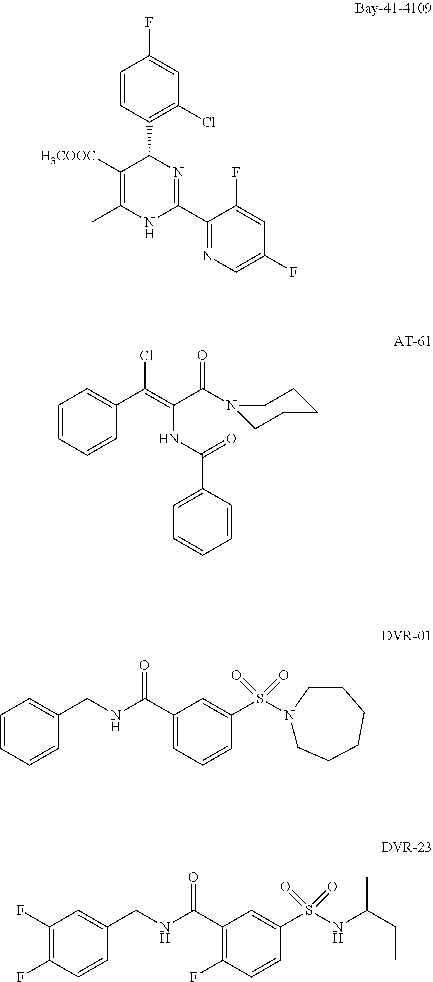

The term capsid inhibitor also includes:

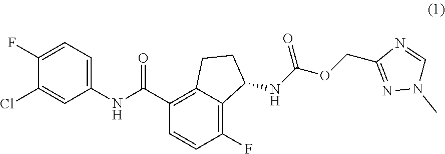 [SMILES: Cn1cnc(COC(=O)N[C@H]2CCc3c(C(=O)Nc4ccc(F)c(Cl)c4)ccc(F)c32)n1]

and a pharmaceutically acceptable salt thereof.

### sAg Secretion Inhibitors/RNA Destabilizers

As described herein the term “sAg secretion inhibitor” includes compounds that are capable of inhibiting, either directly or indirectly, the secretion of sAg (S, M and/or L surface antigens) bearing subviral particles and/or DNA containing viral particles from HBV-infected cells. As used herein, “sAg secretion inhibitors” are also known as “RNA destabilizers”, and these terms are used interchangeably. For example, in certain embodiments, the inhibitor detectably inhibits the secretion of sAg as measured, e.g., using assays known in the art or described herein, e.g., ELISA assay or by Western Blot. In certain embodiments, the inhibitor inhibits the secretion of sAg by at least 5%, at least 10%, at least 20%, at least 50%, at least 75%, or at least 90%. In certain embodiments, the inhibitor reduces serum levels of sAg in a patient by at least 5%, at least 10%, at least 20%, at least 50%, at least 75%, or at least 90%.

The term sAg secretion inhibitor includes compounds described in U.S. Pat. No. 8,921,381, as well as compounds described in United States Patent Application Publication Numbers 2015/0087659 and 2013/0303552. For example, the term includes the compounds PBHBV-001 and PBHBV-2-15, and pharmaceutically acceptable salts thereof:

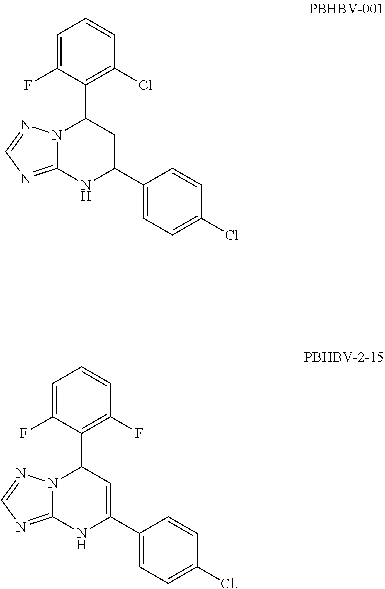 [SMILES: Fc1cccc(Cl)c1C1CC(c2ccc(Cl)cc2)Nc2ncnn21 ; Fc1cccc(F)c1C1C=C(c2ccc(Cl)cc2)Nc2ncnn21]

Specific embodiments of the invention are described below.

In one embodiment, the invention provides a method for preparing a compound of formula 1:

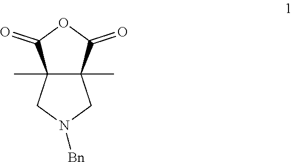 [SMILES: C[C@@]12CN(Cc3ccccc3)C[C@]1(C)C(=O)OC2=O]

comprising reacting a compound of formula 1-1:

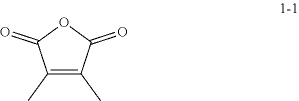

with a compound of formula 1-2:

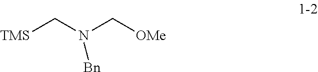 [SMILES: COCN(Cc1ccccc1)C[Si](C)(C)C]

at a temperature of 40° C. or greater to provide the compound of formula 1. The reaction can be carried out neat or in the presence of one or more solvents. In one embodiment, the invention is carried out in a polar aprotic solvent, such as, for example, tetrahydrofuran, 1,2-dichloroethene, methyltetrahydrofuran, toluene, acetonitrile, dimethoxyethane, or carbon tetrachloride. In one embodiment, the reaction is carried out at a temperature in the range from about 0° C. to about 100° C. In another embodiment, the reaction is carried out at a temperature of 60° C. or greater. In another embodiment, the reaction is carried out at a temperature in the range from about 60° C. to about 80° C.

In one embodiment, the invention provides a method for preparing a crystalline form of compound 3:

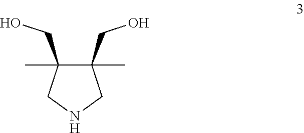

comprising converting a compound of formula 1:

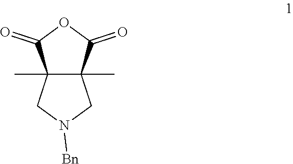 [SMILES: C[C@@]12CN(Cc3ccccc3)C[C@]1(C)C(=O)OC2=O]

to the crystalline form of compound 3 without using column chromatography during the conversion. In one embodiment, the compound can be crystallized from a solvent that comprises dichloromethane or ethyl acetate. In another embodiment, the compound is crystallized from dichloromethane or ethyl acetate.

In one embodiment, the invention provides a crystalline form of compound 3:

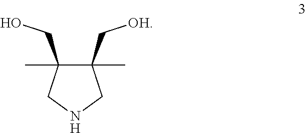

In one embodiment, the invention provides a method for preparing a compound of formula 9:

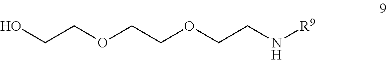

wherein R⁹ is an optionally substituted benzyloxycarbonyl group, comprising converting a compound of formula 8:

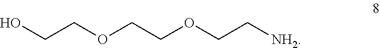

or a salt thereof to the compound of formula 9. The conversion can be carried out at any suitable temperature and can be carried out neat or in the presence of one or more solvents. In one embodiment of the invention the conversion is carried out in a polar or nonpolar aprotic solvent, such as, for example, dichloromethane, chloroform, tetrahydrofuran, methyltetrahydrofuran, carbon tetrachloride, acetonitrile, pyridine, dimethylformamide, dimethylacetamide, or toluene. In one embodiment, the conversion is carried out at a temperature in the range from about 0° C. to about 100° C. In another embodiment, the conversion is carried out at a temperature in the range from about 15° C. to about 25° C. In one embodiment, R⁹ is benzyloxycarbonyl or nitrobenzyloxycarbonyl. In one embodiment, the compound of formula 8 is converted to the compound of formula 9 by treating the compound of formula 8 with benzyloxycarbonyl chloride in a suitable solvent in the presence of a suitable base. In one embodiment, the base is an amine base, such as, for example, trimethylamine, triethylamine, pyridine, dimethylaminopyridine, diisopropylethylamine, or tripropyl amine.

In one embodiment, the invention provides a method for preparing a compound of formula 10:

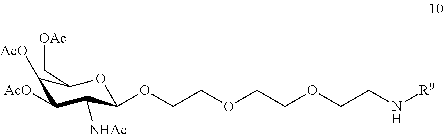

wherein R⁹ is an optionally substituted benzyloxycarbonyl group, comprising converting a corresponding compound of formula 9:

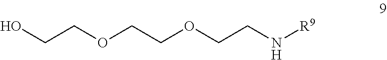

to the compound of formula 10. The conversion can be carried out at any suitable temperature and can be carried out neat or in the presence of one or more solvents. In one embodiment, the conversion provides the compound of formula 10 as at least about 85%, 90%, or 95% the beta-isomer. In one embodiment of the invention the conversion is carried out in a nonpolar aprotic solvent, such as, for example, dichloroethane, dichloromethane, acetonitrile, methyltetrahydrofuran, tetrahydrofuran, dimethoxyethane, or toluene. In one embodiment, the conversion is carried out at a temperature in the range from about 0° C. to about 100° C. In another embodiment, the conversion is carried out at a temperature in the range from about 80° C. to about 85° C. In another embodiment, the conversion is carried out at a temperature in the range from about 35° C. to about 45° C. In another embodiment, the conversion is carried out at a temperature in the range from about 45° C. to about 55° C. In another embodiment, the conversion is carried out at a temperature in the range from about 55° C. to about 65° C. In another embodiment, the conversion is carried out at a temperature that optimizes the beta:alpha ratio of the product. In one embodiment, R⁹ is benzyloxycarbonyl or nitrobenzyloxycarbonyl. In one embodiment, the compound of formula 9 is converted to the compound of formula 10 by treatment with a compound of formula 7:

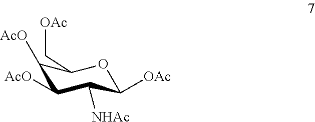

in the presence of a suitable catalyst and a suitable solvent. In one embodiment, the catalyst is Sc(OTf)₃, trimethylsilyl trifluoromethanesulfonate, zinc chloride, or 4A molecular sieves.

In one embodiment, the invention provides a method for preparing a compound of formula 10:

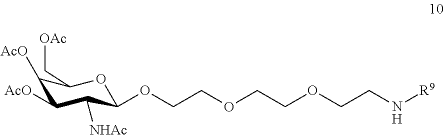

wherein R⁹ is an optionally substituted benzyloxycarbonyl group, comprising converting a compound of formula 8:

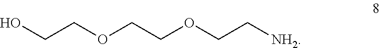

or a salt thereof to a corresponding compound of formula 9;

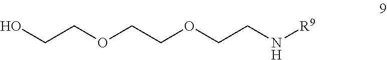

and subsequently converting the corresponding compound of formula 9 to the compound of formula 10, without purifying the compound of formula 9 by chromatography.

In one embodiment, the invention provides a method for preparing a salt of formula 11:

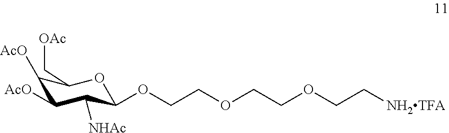

comprising treating a compound of formula 10:

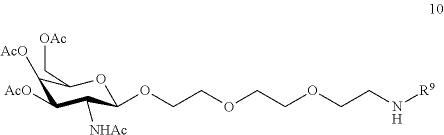

wherein R⁹ is an optionally substituted benzyloxycarbonyl group, with hydrogen and trifluoroacetic acid in the presence of a suitable catalyst and in the presence of a suitable solvent.

In one embodiment, the suitable catalyst comprises palladium on carbon. In one embodiment, the suitable solvent comprises tetrahydrofuran. The reaction can be carried out at any suitable temperature. In one embodiment, the reaction is carried out at a temperature in the range from about 0° C. to about 50° C. In another embodiment, the reaction is carried out at a temperature in the range from about 20° C. to about 25° C. In one embodiment, R⁹ is benzyloxycarbonyl or nitrobenzyloxycarbonyl.

In one embodiment, the invention provides a method for preparing a compound of formula 15D:

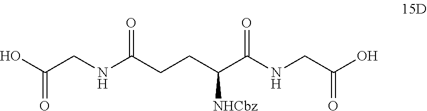 [SMILES: O=C(O)CNC(=O)CC[C@H](NC(=O)OCc1ccccc1)C(=O)NCC(=O)O]

or a salt thereof, comprising converting a compound of formula 15C:

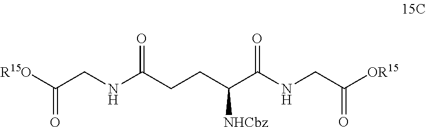

wherein each R¹⁵ is a (C₁-C₆)alkyl, to the compound of formula 15D or the salt thereof. The conversion can be carried out at any suitable temperature and can be carried out neat or in the presence of one or more solvents. In one embodiment of the invention, the conversion is carried out in a polar protic solvent, such as, for example, methanol, ethanol, tetrahydrofuran, and/or water. In one embodiment, the conversion is carried out at a temperature in the range from about 0° C. to about 100° C. In another embodiment, the conversion is carried out at a temperature in the range from about 15° C. to about 25° C. In one embodiment, the conversion is carried out in the presence of a suitable base, such as, for example, sodium hydroxide, lithium hydroxide, or potassium hydroxide.

In one embodiment, the invention provides a method for preparing a compound of formula 15C:

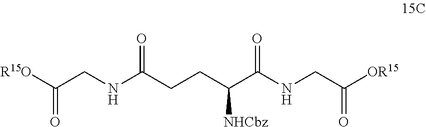

wherein each R¹⁵ is a (C₁-C₆)alkyl, comprising reacting a compound of formula 15A:

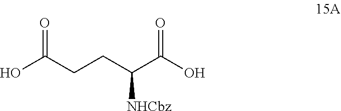 [SMILES: O=C(O)CC[C@H](NC(=O)OCc1ccccc1)C(=O)O]

or a salt thereof, with a corresponding compound of formula 15B:

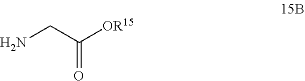

or a salt thereof, to provide the compound of formula 15C. The reaction can be carried out at any suitable temperature and can be carried out neat or in the presence of one or more solvents. In one embodiment of the invention the reaction is carried out in a polar aprotic solvent, such as, for example, dimethylformamide, dichloromethane, 1,2-dichloroethane, or dimethylacetamide. In one embodiment, the reaction is carried out at a temperature in the range from about 0° C. to about 50° C. In another embodiment, the reaction is carried out at a temperature in the range from about 5° C. to about 10° C. In one embodiment, the reaction is carried out in the presence of a suitable base. In one embodiment, the base is a hindered amine base, such as, for example, diisopropylethylamine, trimethylamine, pyridine, or dimethylaminopyridine. In one embodiment, the reaction is carried out in the presence of a suitable coupling agent, such as, for example, 1-ethyl-3-(3-dimethylaminopropyl)carbodiimide EDC, N,N′-dicyclohexyl-carbodiimide DCC, (1-[bis(dimethylamino)methylene]-1H-1,2,3-triazolo[4,5-b]pyridinium 3-oxide hexafluorophosphate HATU, (2-(1H-benzotriazol-1-yl)-1,1,3,3-tetramethyluronium hexafluorophosphate HBTU, or propanephosphonic acid anhydride T3P).

In one embodiment, the invention provides a method for preparing a compound of formula 13A:

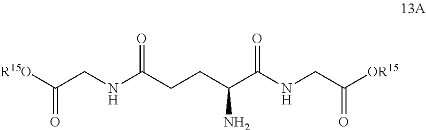

wherein each R¹⁵ is a (C₁-C₆)alkyl, comprising converting a corresponding compound of formula 15C:

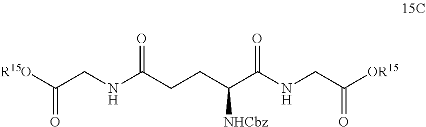

wherein each R¹⁵ is a (C₁-C₆)alkyl, to the compound of formula 13A. The conversion can be carried out at any suitable temperature and can be carried out neat or in the presence of one or more solvents. In one embodiment of the invention, the conversion is carried out in a polar protic solvent, such as, for example, methanol, ethyl acetate, tetrahydrofuran, methyltetrahydrofuran, or ethanol. In one embodiment, the conversion is carried out at a temperature in the range from about 0° C. to about 100° C. In another embodiment, the conversion is carried out at a temperature in the range from about 15° C. to about 25° C. In one embodiment, the conversion is carried out in the presence of a suitable catalyst, such as, for example, palladium on carbon or Pd(OH)₂.

In one embodiment, the invention provides a method for preparing a compound of formula 13B:

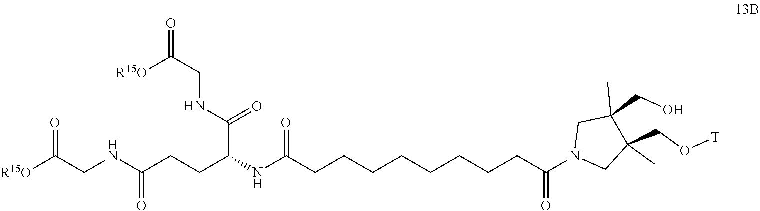

wherein each R¹⁵ is a (C₁-C₆)alkyl and T is an optionally substituted triphenylmethyl group, comprising converting a corresponding compound of formula 13A:

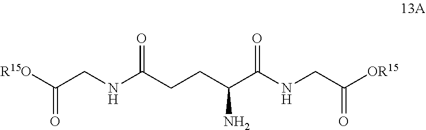

to the compound of formula 13B. The conversion can be carried out at any suitable temperature and can be carried out neat or in the presence of one or more solvents. In one embodiment of the invention, the conversion is carried out in a nonpolar aprotic solvent, such as, for example, dichloromethane, 1,2-dichloroethane, dimethylformamide, or dimethylacetamide. In one embodiment, the conversion is carried out at a temperature in the range from about −78° C. to about 100° C. In another embodiment, the conversion is carried out at a temperature in the range from about 0° C. to about 30° C. In one embodiment, the conversion is carried out in the presence of a suitable coupling agent, such as, for example, 1-ethyl-3-(3-dimethylamino-propyl)carbodiimide EDC, N,N′-dicyclohexylcarbodiimide DCC, (1-[bis(dimethylamino)-methylene]-1H-1,2,3-triazolo[4,5-b]pyridinium 3-oxide hexafluorophosphate HATU, (2-(1H-benzotriazol-1-yl)-1,1,3,3-tetramethyluronium hexafluorophosphate HBTU, or propanephosphonic acid anhydride T3P). In one embodiment, the compound of formula 13A is converted to the compound of formula 13B, by treating the compound of formula 13A with a corresponding compound of formula 6:

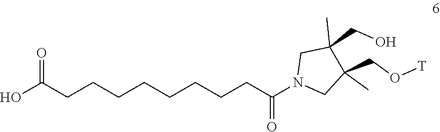

or a salt thereof, wherein DMTr is 4,4-dimethoxytriphenylmethyl under suitable amide forming conditions. In one embodiment, the compound of formula 13A is treated with the compound of formula:

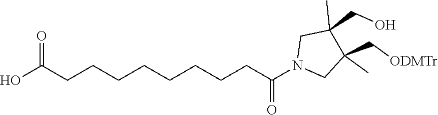

in dichloromethane at a temperature in the range from about 0° C. to about 30° C. in the presence of 1-ethyl-3-(3-dimethylaminopropyl)carbodiimide.

In one embodiment, the invention provides a method for preparing a compound of formula 13C:

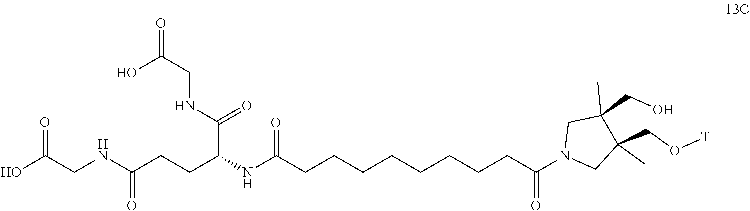

comprising converting a compound of formula 13B:

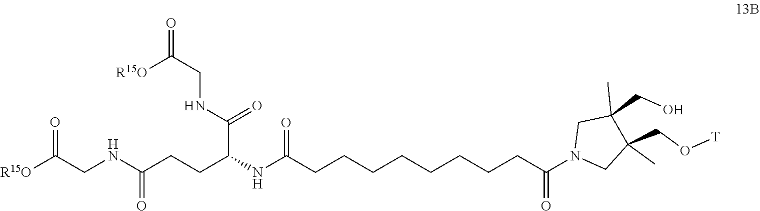

wherein each R¹⁵ is a (C₁-C₆)alkyl and T is an optionally substituted triphenylmethyl group, to the compound of formula 13C. The conversion can be carried out at any suitable temperature and can be carried out neat or in the presence of one or more solvents. In one embodiment of the invention, the conversion is carried out in a polar protic solvent, such as, for example, methanol, ethanol, tetrahydrofuran, and/or water. In one embodiment, the conversion is carried out at a temperature in the range from about 0° C. to about 100° C. In another embodiment, the conversion is carried out at a temperature in the range from about 20° C. to about 40° C. In one embodiment, the conversion is carried out in the presence of a suitable base, such as, for example, potassium hydroxide, lithium hydroxide, or sodium hydroxide. In one embodiment, the compound of formula 13B is converted to the compound of formula 13C by treatment with potassium hydroxide in a solvent comprising methanol and water.

In one embodiment, the invention provides a method for preparing a crystalline potassium salt of a compound of formula 13CC:

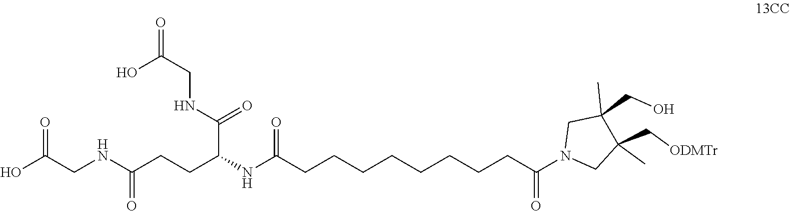

comprising treating a compound of formula 13CC or a salt thereof with potassium hydroxide in methanol. In one embodiment, the crystalline potassium salt of a compound of formula 13CC can be prepared as described in Example 30.

In one embodiment, the invention provides a method for preparing a compound of formula 11B:

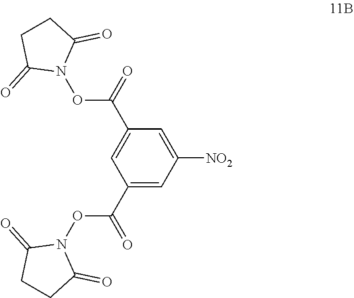 [SMILES: O=C(ON1C(=O)CCC1=O)c1cc(C(=O)ON2C(=O)CCC2=O)cc([N+](=O)[O-])c1]

comprising converting a compound of formula 11A:

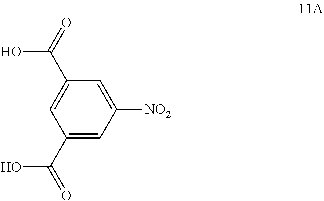 [SMILES: O=C(O)c1cc(C(=O)O)cc([N+](=O)[O-])c1]

or a salt thereof, to the compound of formula 11B. The conversion can be carried out at any suitable temperature and can be carried out neat or in the presence of one or more solvents. In one embodiment of the invention the conversion is carried out in a nonpolar aprotic solvent, such as, for example, dichloromethane, 1,2-dichloroethane, methyltetrahydrofuran, tetrahydrofuran, dimethylformamide, or dimethylacetamide. In one embodiment, the conversion is carried out at a temperature in the range from about 0° C. to about 100° C. In another embodiment, the conversion is carried out at a temperature in the range from about 5° C. to about 30° C. In one embodiment, the compound of formula 11A is converted to the compound of formula 11B by treating the compound of formula 11A or the salt thereof with 1-ethyl-3-(3-dimethylaminopropyl)carbodiimide EDC, N,N′-dicyclohexylcarbodiimide DCC, (1-[bis(dimethylamino)methylene]-1H-1,2,3-triazolo[4,5-b]pyridinium 3-oxide hexafluorophosphate HATU, (2-(1H-benzotriazol-1-yl)-1,1,3,3-tetramethyluronium hexafluorophosphate HBTU, or propanephosphonic acid anhydride T3P) in dichloromethane.

In one embodiment, the invention provides a method for preparing a compound of formula 12:

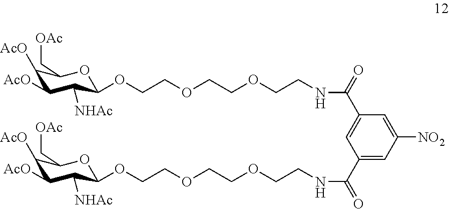

comprising converting a compound of formula 11B:

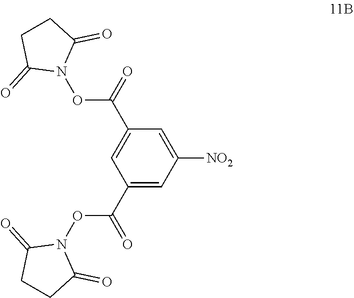 [SMILES: O=C(ON1C(=O)CCC1=O)c1cc(C(=O)ON2C(=O)CCC2=O)cc([N+](=O)[O-])c1]

to the compound of formula 12. The conversion can be carried out at any suitable temperature and can be carried out neat or in the presence of one or more solvents. In one embodiment of the invention the conversion is carried out in a nonpolar aprotic solvent, such as, for example, dichloromethane, 1,2-dichloroethane, methyltetrahydrofuran, tetrahydrofuran, dimethylformamide, or dimethylacetamide. In one embodiment, the conversion is carried out at a temperature in the range from about 0° C. to about 50° C. In another embodiment, the conversion is carried out at a temperature in the range from about 0° C. to about 30° C. In one embodiment, the conversion is carried out in the presence of a suitable base. In one embodiment, the base is a hindered amine base, such as, for example, diisopropylethylamine, trimethylamine, dimethylaminopyridine, or pyridine. In one embodiment, the compound of formula 11B is converted to the compound of formula 12 by treating the compound of formula 11B with a compound of formula 11:

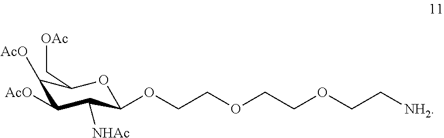

or a salt thereof, in the presence of a suitable base and a suitable solvent. In one embodiment, the compound of formula 11B is converted to the compound of formula 12 by treating the compound of formula 11B with the trifloroacetic acid salt of a compound of formula 11:

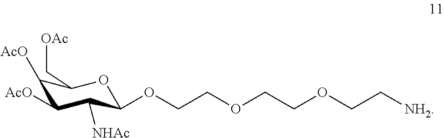

in the presence of diisopropylethylamine and a solvent that comprises dichloromethane.

In one embodiment, the invention provides a method for preparing a compound of formula 13:

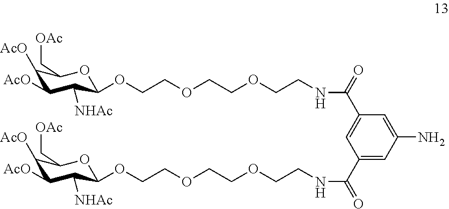

or a salt thereof, comprising reducing a compound of formula 12:

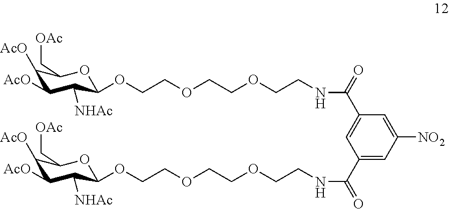

to provide the compound of formula 13 or the salt thereof. The reduction can be carried out at any suitable temperature and can be carried out neat or in the presence of one or more solvents. In one embodiment of the invention the reduction is carried out in a polar aprotic solvent, such as, for example, tetrahydrofuran, methyltetrahydrofuran, or ethyl acetate. In one embodiment, the reduction is carried out at a temperature in the range from about 0° C. to about 50° C. In another embodiment, the reduction is carried out at a temperature in the range from about 0° C. to about 30° C. In one embodiment, the reduction is carried out in the presence of a suitable catalyst, such as, for example, palladium on carbon. In one embodiment, the compound of formula 13 or the salt thereof, is a trifluoroacetic acid salt of the following formula:

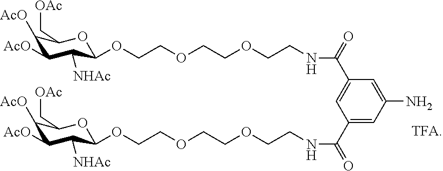

In one embodiment, the invention provides a method for preparing a compound of formula 14:

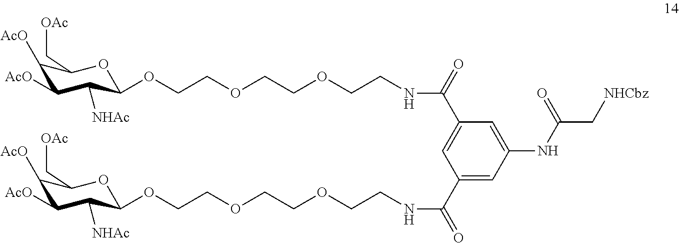

comprising converting a compound of formula 13:

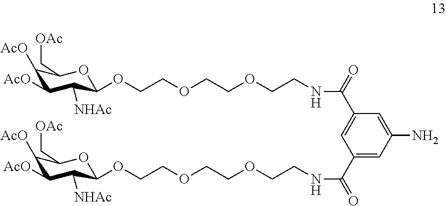

or a salt thereof to the compound of formula 14. The conversion can be carried out at any suitable temperature and can be carried out neat or in the presence of one or more solvents. In one embodiment of the invention the conversion is carried out in a nonpolar aprotic solvent, such as, for example, dichloromethane, 1,2-dichloroethane, methyltetrahydrofuran, tetrahydrofuran, dimethylformamide, or dimethylacetamide. In one embodiment, the conversion is carried out at a temperature in the range from about −78° C. to about 25° C. In another embodiment, the conversion is carried out at a temperature in the range from about −25° C. to about 30° C. In one embodiment, the conversion is carried out in the presence of a suitable base. In one embodiment, the base is an amine base, such as, for example, trimethylamine, triethylamine, diisopropylethylamine, dimethylaminopyridine, pyridine, or tripropylamine. In one embodiment, the conversion is carried out in the presence of a suitable coupling reagent, such as, for example, propanephosphonic acid anhydride. In one embodiment, the compound of formula 13 is converted to the compound of formula 14, by treating the compound of formula 13 with a compound of formula:

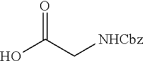 [SMILES: O=C(O)CNC(=O)OCc1ccccc1]

or a salt thereof, in a solvent comprising dichloromethane in the presence of a coupling agent, such as, for example, 1-ethyl-3-(3-dimethylaminopropyl)carbodiimide EDC, (1-[bis(dimethylamino)methylene]-1H-1,2,3-triazolo[4,5-b]pyridinium 3-oxide hexafluorophosphate HATU, (2-(1H-benzotriazol-1-yl)-1,1,3,3-tetramethyluronium hexafluorophosphate HBTU, or propanephosphonic acid anhydride T3P, at a temperature n the range from about −15° C. to about 15° C.

In one embodiment, the invention provides a method for preparing a compound of formula 16:

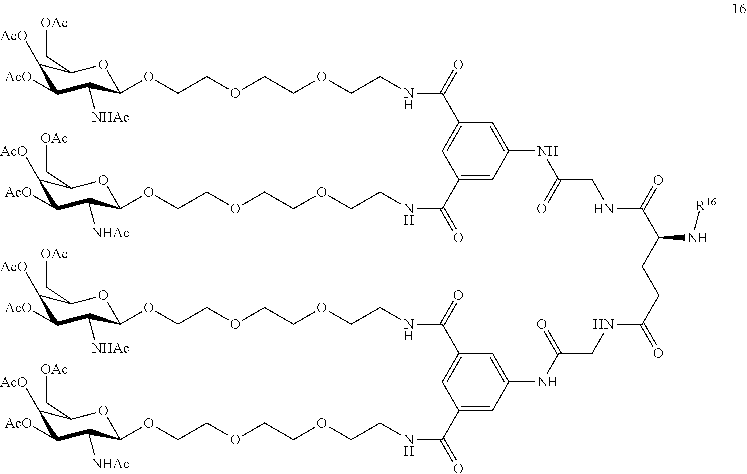

wherein R¹⁶ is an amine protecting group, comprising converting a compound of formula 13:

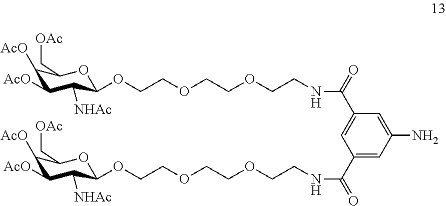

or a salt thereof, to the compound of formula 16. The conversion can be carried out at any suitable temperature and can be carried out neat or in the presence of one or more solvents. In one embodiment of the invention the conversion is carried out in a nonpolar aprotic solvent, such as, for example, dichloromethane, 1,2-dichloroethane, methyltetrahydrofuran, tetrahydrofuran, dimethylformamide, or dimethylacetamide. In one embodiment, the conversion is carried out at a temperature in the range from about −78° C. to about 50° C. In another embodiment, the conversion is carried out at a temperature in the range from about −25° C. to about 50° C. In one embodiment, the conversion is carried out in the presence of a suitable base. In one embodiment, the base is an amine base, such as, for example, trimethylamine, triethylamine, or tripropylamine, diisopropylethylamine, dimethylaminopyridine, or pyridine. In one embodiment, the conversion is carried out in the presence of a suitable coupling reagent, such as, for example, 1-ethyl-3-(3-dimethyl aminopropyl)carbodiimide EDC, (1-[bis(dimethylamino)methylene]-1H-1,2,3-triazolo[4,5-b]pyridinium 3-oxide hexafluorophosphate HATU, (2-(1H-benzotriazol-1-yl)-1,1,3,3-tetramethyluronium hexafluorophosphate HBTU, or propanephosphonic acid anhydride T3P. In one embodiment, the compound of formula 13 or the salt thereof is converted to the compound of formula 16, by treating the compound of formula 13 with a corresponding compound of formula 15DD:

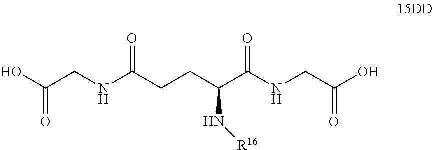

wherein R¹⁶ is an amine protecting group, or a salt thereof, under suitable coupling conditions. In one embodiment, a trifluoroacetic acid salt of a compound of formula 13:

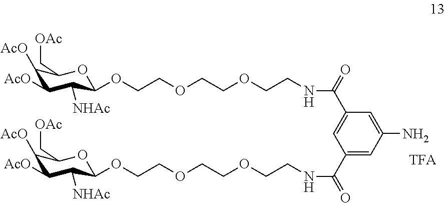

is treated with a compound of formula 15D wherein R¹⁶ is benzyloxycarbonyl:

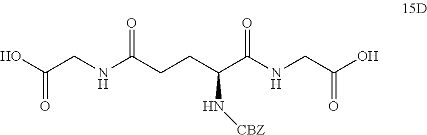

under suitable coupling conditions to provide a compound of formula 16 wherein R¹⁶ is benzyloxycarbonyl. In one embodiment, the compound of formula 13 is treated with the compound of formula 15D or 15DD in the presence of propanephosphonic acid anhydride, trimethylamine, and a solvent comprising dichloromethane to provide the compound of formula 16.

In one embodiment, the invention provides a method for preparing a compound of formula 18:

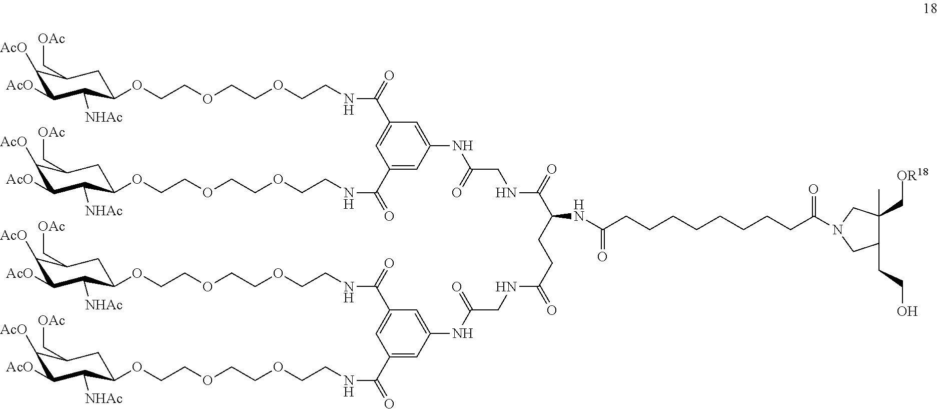

wherein V is a suitable protecting group, comprising converting a compound of formula 13:

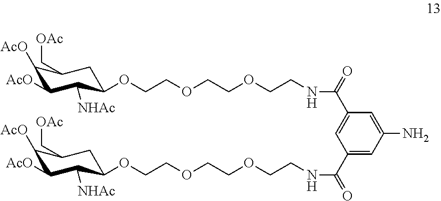

or a salt thereof, to the compound of formula 18. The conversion can be carried out at any suitable temperature and can be carried out neat or in the presence of one or more solvents. In one embodiment of the invention the conversion is carried out in a nonpolar aprotic solvent, such as, for example, dichloromethane, 1,2-dichloroethane, methyltetrahydrofuran, tetrahydrofuran, dimethylformamide, or dimethylacetamide. In one embodiment, the conversion is carried out at a temperature in the range from about −78° C. to about 50° C. In another embodiment, the conversion is carried out at a temperature in the range from about −25° C. to about 50° C. In one embodiment, the conversion is carried out in the presence of a suitable base. In one embodiment, the base is an amine base, such as, for example, trimethylamine, triethylamine, or tripropylamine, diisopropylethylamine, dimethylaminopyridine, or pyridine. In one embodiment, the conversion is carried out in the presence of a suitable coupling reagent, such as, for example, 1-ethyl-3-(3-dimethylaminopropyl)carbodiimide EDC, (1-[bis(dimethylamino)methylene]-1H-1,2,3-triazolo[4,5-b]pyridinium 3-oxide hexafluorophosphate HATU, (2-(1H-benzotriazol-1-yl)-1,1,3,3-tetramethyluronium hexafluorophosphate HBTU, or propanephosphonic acid anhydride T3P. In one embodiment, the compound of formula 13 or the salt thereof is converted to the compound of formula 18, by treating the compound of formula 13 or the salt thereof with a compound of formula 13CCC:

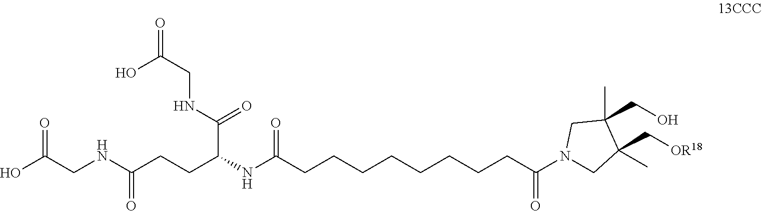

wherein R¹⁸ is a suitable protecting group, or a salt thereof, under suitable coupling conditions. In one embodiment, a trifluoroacetic acid salt of a compound of formula 13:

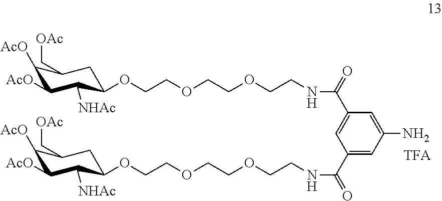

is treated with a compound of formula 13CCC, wherein V is 4,4-dimethoxytriphenylmethyl under suitable coupling conditions to provide a compound of formula 18:

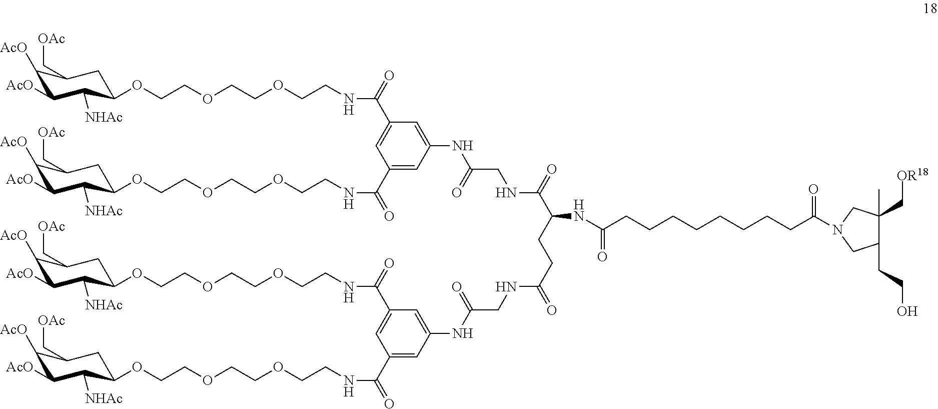

wherein R¹⁸ is 4,4-dimethoxytriphenylmethyl. In one embodiment, the compound of formula 13 is treated with the compound of formula 13CCC in the presence of propanephosphonic acid anhydride, trimethylamine, and a solvent comprising dichloromethane to provide the compound of formula 18.

In one embodiment, the invention provides a method for preparing a compound of formula 16-2:

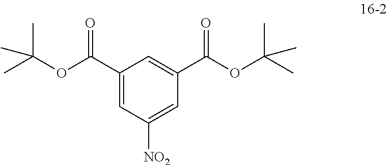 [SMILES: CC(C)(C)OC(=O)c1cc(C(=O)OC(C)(C)C)cc([N+](=O)[O-])c1]

comprising converting a compound of formula 16-1:

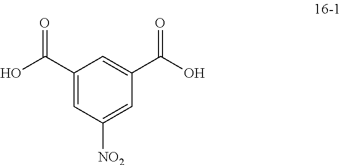 [SMILES: O=C(O)c1cc(C(=O)O)cc([N+](=O)[O-])c1]

or a salt thereof, to the compound of formula 16-2. The conversion can be carried out at any suitable temperature and can be carried out neat or in the presence of one or more solvents. In one embodiment of the invention, the conversion is carried out in a nonpolar aprotic solvent, such as, for example, dichloromethane, 1,2-dichloroethane, chloroform, or carbon tetrachloride. In one embodiment, the conversion is carried out at a temperature in the range from about −78° C. to about 100° C. In another embodiment, the conversion is carried out at a temperature in the range from about −0° C. to about 30° C. In one embodiment, the conversion is carried out by activating the carboxylic acid groups in the compound of formula 16-1, for example, by treating the compound of formula 16-1 with oxalyl chloride, and treating the resulting carboxylic acid chloride groups with tert-butanol to provide the compound of formula 16-2.

In one embodiment, the invention provides a method for preparing a compound of formula 16-3:

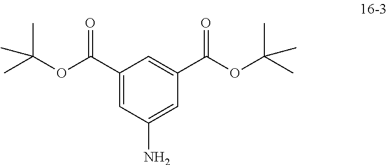 [SMILES: CC(C)(C)OC(=O)c1cc(N)cc(C(=O)OC(C)(C)C)c1]

comprising converting a compound of formula 16-2:

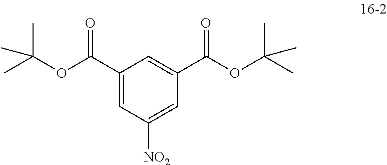 [SMILES: CC(C)(C)OC(=O)c1cc(C(=O)OC(C)(C)C)cc([N+](=O)[O-])c1]

to the compound of formula 16-3. The conversion can be carried out at any suitable temperature and can be carried out neat or in the presence of one or more solvents. In one embodiment of the invention the conversion is carried out in a polar protic solvent, such as, for example, methanol or ethanol. In one embodiment, the reaction is carried out at a temperature in the range from about −78° C. to about 50° C. In another embodiment, the conversion is carried out at a temperature in the range from about −0° C. to about 50° C. In one embodiment, the conversion is carried out in the presence of a suitable catalyst, such as, for example, palladium on carbon.

In one embodiment, the invention provides a method for preparing a compound of formula 16-4:

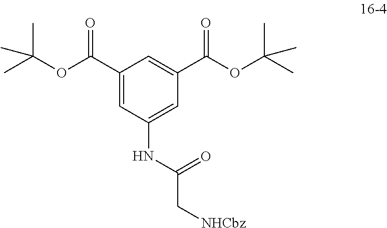 [SMILES: CC(C)(C)OC(=O)c1cc(NC(=O)CNC(=O)OCc2ccccc2)cc(C(=O)OC(C)(C)C)c1]

comprising converting a compound of formula 16-3:

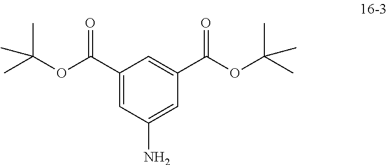 [SMILES: CC(C)(C)OC(=O)c1cc(N)cc(C(=O)OC(C)(C)C)c1]

to the compound of formula 16-4. The conversion can be carried out at any suitable temperature and can be carried out neat or in the presence of one or more solvents. In one embodiment of the invention the conversion is carried out in a nonpolar aprotic solvent, such as, for example, dichloromethane, 1,2-dichloroethane, methyltetrahydrofuran, tetrahydrofuran, dimethylformamide, or dimethylacetamide. In one embodiment, the conversion is carried out at a temperature in the range from about −78° C. to about 50° C. In another embodiment, the conversion is carried out at a temperature in the range from about −0° C. to about 50° C. In one embodiment, the conversion is carried out in the presence of a suitable base. In one embodiment, the base is an amine base, such as, for example, trimethylamine, triethylamine, or tripropylamine, diisopropylethylamine, dimethylaminopyridine, or pyridine. In one embodiment, the conversion is carried out in the presence of a suitable coupling reagent, such as, for example, 1-ethyl-3-(3-dimethylaminopropyl)carbodiimide EDC, (1-[bis(dimethylamino)methylene]-1H-1,2,3-triazolo[4,5-b]pyridinium 3-oxide hexafluorophosphate HATU, (2-(1H-benzotriazol-1-yl)-1,1,3,3-tetramethyluronium hexafluorophosphate HBTU, or propanephosphonic acid anhydride T3P.

In one embodiment, the invention provides a method for preparing a compound of formula 16-5:

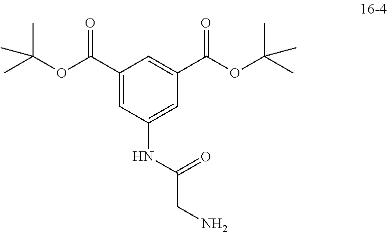 [SMILES: CC(C)(C)OC(=O)c1cc(NC(=O)CN)cc(C(=O)OC(C)(C)C)c1]

or a salt thereof, comprising converting a compound of formula 16-4:

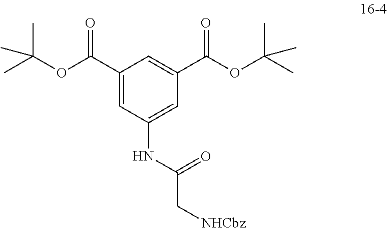 [SMILES: CC(C)(C)OC(=O)c1cc(NC(=O)CNC(=O)OCc2ccccc2)cc(C(=O)OC(C)(C)C)c1]

to the compound of formula 16-5. The conversion can be carried out at any suitable temperature and can be carried out neat or in the presence of one or more solvents. In one embodiment of the invention the conversion is carried out in a polar protic solvent, such as, for example, methanol, ethanol, tetrahydrofuran, or ethyl acetate. In one embodiment, the conversion is carried out at a temperature in the range from about −78° C. to about 50° C. In another embodiment, the conversion is carried out at a temperature in the range from about −0° C. to about 50° C. In one embodiment, the conversion is carried out in the presence of a suitable catalyst, such as, for example, palladium on carbon.

In one embodiment, the invention provides a method for preparing a compound of formula 16D:

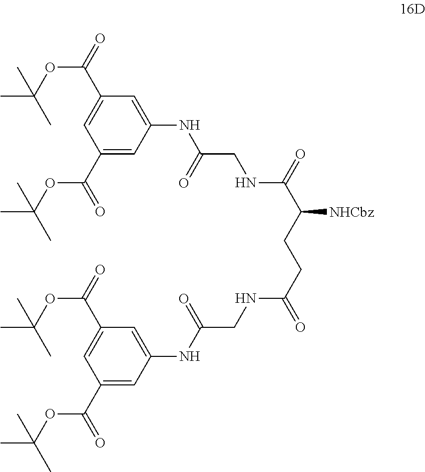 [SMILES: CC(C)(C)OC(=O)c1cc(NC(=O)CNC(=O)CC[C@H](NC(=O)OCc2ccccc2)C(=O)NCC(=O)Nc2cc(C(=O)OC(C)(C)C)cc(C(=O)OC(C)(C)C)c2)cc(C(=O)OC(C)(C)C)c1]

or a salt thereof, comprising converting a compound of formula 16-5:

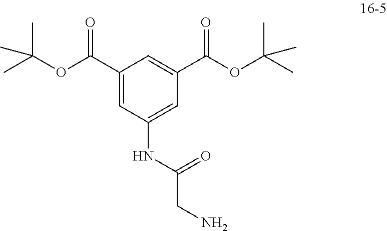 [SMILES: CC(C)(C)OC(=O)c1cc(NC(=O)CN)cc(C(=O)OC(C)(C)C)c1]

to the compound of formula 16D. The conversion can be carried out at any suitable temperature and can be carried out neat or in the presence of one or more solvents. In one embodiment of the invention the conversion is carried out in a nonpolar aprotic solvent, such as, for example, dichloromethane, 1,2-dichloroethane, methyltetrahydrofuran, tetrahydrofuran, dimethylformamide, or dimethylacetamide. In one embodiment, the conversion is carried out at a temperature in the range from about −78° C. to about 50° C. In another embodiment, the conversion is carried out at a temperature in the range from about 0° C. to about 50° C. In one embodiment, the conversion is carried out in the presence of a suitable base. In one embodiment, the base is an amine base, such as, for example, trimethylamine, triethylamine, or tripropylamine, diisopropylethylamine, dimethylaminopyridine, or pyridine. In one embodiment, the conversion is carried out in the presence of a suitable coupling reagent, such as, for example, 1-ethyl-3-(3-dimethylaminopropyl)carbodiimide EDC, (1-[bis(dimethyl-amino)methylene]-1H-1,2,3-triazolo[4,5-b]pyridinium 3-oxide hexafluorophosphate HATU, (2-(1H-benzotriazol-1-yl)-1,1,3,3-tetramethyluronium hexafluorophosphate HBTU, or propanephosphonic acid anhydride T3P.

In one embodiment, the invention provides a method for preparing a compound of formula 16E:

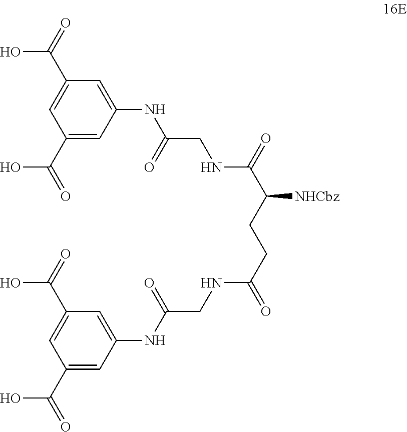 [SMILES: O=C(CC[C@H](NC(=O)OCc1ccccc1)C(=O)NCC(=O)Nc1cc(C(=O)O)cc(C(=O)O)c1)NCC(=O)Nc1cc(C(=O)O)cc(C(=O)O)c1]

or a salt thereof, comprising converting a compound of formula 16D:

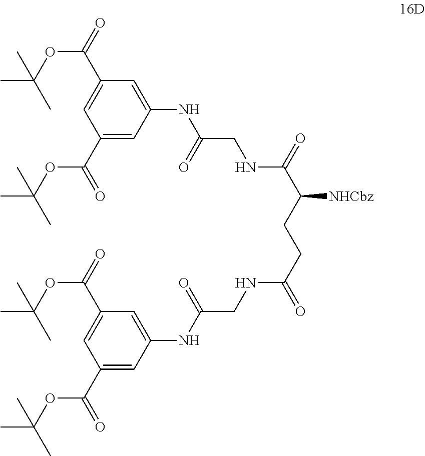 [SMILES: CC(C)(C)OC(=O)c1cc(NC(=O)CNC(=O)CC[C@H](NC(=O)OCc2ccccc2)C(=O)NCC(=O)Nc2cc(C(=O)OC(C)(C)C)cc(C(=O)OC(C)(C)C)c2)cc(C(=O)OC(C)(C)C)c1]

or a salt thereof, to the compound of formula 16D. The conversion can be carried out at any suitable temperature and can be carried out neat or in the presence of one or more solvents. In one embodiment of the invention the conversion is carried out in a nonpolar aprotic solvent, such as, for example, dichloromethane, chloroform, or carbon tetrachloride. In one embodiment, the conversion is carried out at a temperature in the range from about −25° C. to about 50° C. In another embodiment, the conversion is carried out at a temperature in the range from about 0° C. to about 50° C. In one embodiment, the conversion is carried out in the presence of a suitable acid. In one embodiment, the acid is trifluoroacetic acid.

In one embodiment, the invention provides a method for preparing a compound of formula 16:

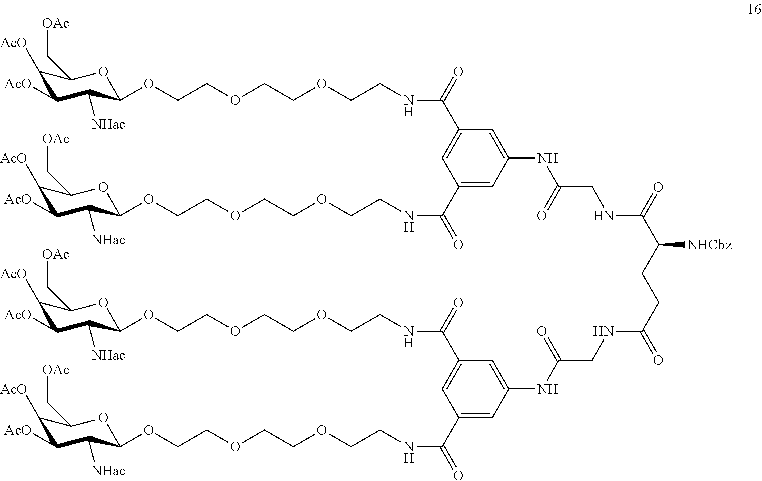

or a salt thereof, comprising converting a compound of formula 16E:

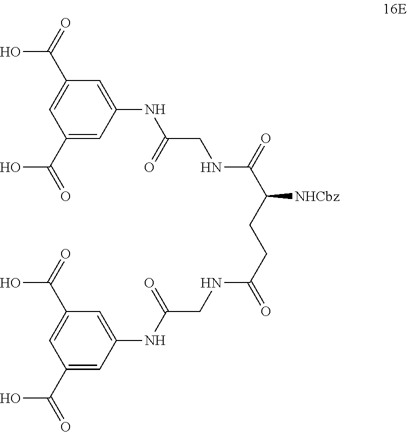 [SMILES: O=C(CC[C@H](NC(=O)OCc1ccccc1)C(=O)NCC(=O)Nc1cc(C(=O)O)cc(C(=O)O)c1)NCC(=O)Nc1cc(C(=O)O)cc(C(=O)O)c1]

or a salt thereof, to the compound of formula 16. The conversion can be carried out at any suitable temperature and can be carried out neat or in the presence of one or more solvents. In one embodiment of the invention the conversion is carried out in a polar aprotic solvent, such as, for example, dimethylformamide, dichloromethane, or dimethylaminopyridine. In one embodiment, the conversion is carried out at a temperature in the range from about −25° C. to about 25° C. In another embodiment, the conversion is carried out at a temperature in the range from about 0° C. to about 10° C. In one embodiment, the conversion is carried out in the presence of a suitable base. In one embodiment, the base is a hindered amine base, such as, for example, diisopropylethylamine, trimethylamine, dimethylaminopyridine, or pyridine. In one embodiment, the conversion is carried out in the presence of a suitable coupling agent, such as, for example, 1-ethyl-3-(3-dimethylaminopropyl)carbodimide EDC. In one embodiment, the conversion is carried out in the presence of a suitable hydroxybenzotriazole, N,N′-dicyclohexylcarbodiimide DCC, (1-[bis(dimethylamino)-methylene]-1H-1,2,3-triazolo[4,5-b]pyridinium 3-oxide hexafluorophosphate HATU, or propanephosphonic acid anhydride T3P). In one embodiment, the compound of formula 16E or the salt thereof is converted to the compound of formula 16 or the salt thereof, by reacting the compound of formula 16E or the salt thereof with a compound of formula 11:

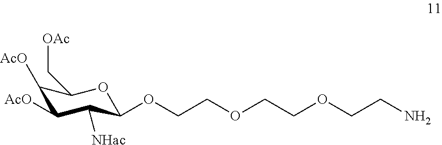

or a salt thereof, under suitable coupling conditions.

Compounds of formula 16 and formula 18 can be used to prepare therapeutic conjugates, including the corresponding therapeutic conjugates described in International Patent Application Publication Number WO 2018/191278.

The invention will now be illustrated by the following non-limiting examples.

### EXAMPLES

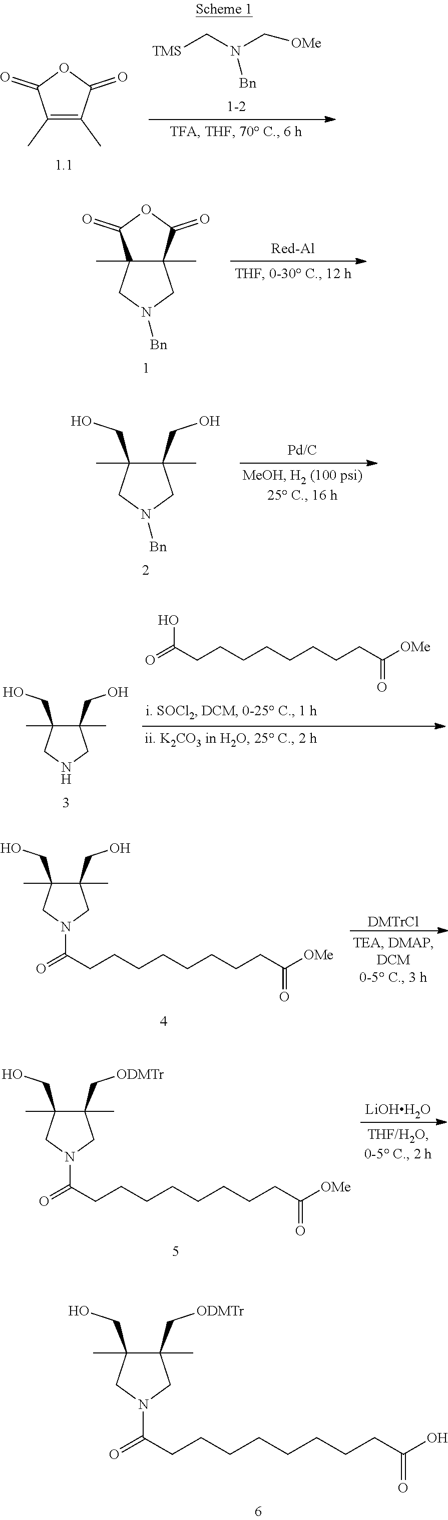

For Compounds 1-6, the bold-wedge bonds indicate a cis-isomer, not absolute stereochemistry. The invention provides Compounds 1-6 having both cis-configurations. When Compounds 1-6 are incorporated into other compounds herein, the bold-wedge bonds from Compounds 1-6 indicate a cis-conformation, while any other bold, bold-wedge, dashed or dashed-wedge bonds therein indicate absolute stereochemistry.

### Example 1. Synthesis of Compound 3

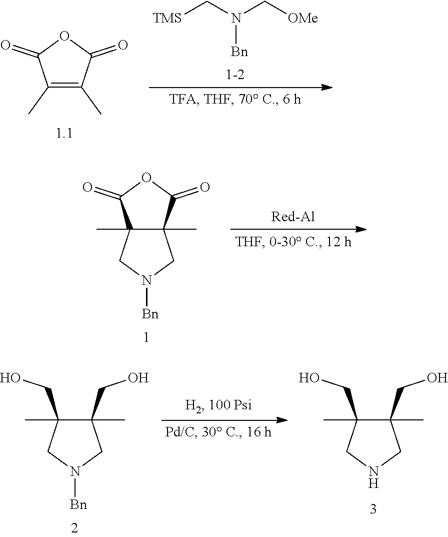

To a solution of compound 1-1 (200 g, 1.58 mol) in 2-MeTHF (2.4 L) was added trifluoroacetic acid TFA (5.4 g, 4.7 mmol). The reaction mixture was heated to 65-70° C. to 70° C. and compound 1-2 (414 g, 1.74 mol) was added slowly maintaining reaction temperature 65-70° C. After completion of addition, the reaction mixture was heated at 65° C. to 70° C. for not less than 2 hours until completion of reaction as confirmed by UPLC (disappearance of compound 1-1). The reaction mixture was then cooled to −5 to 0° C. Red-Al (1.6 Kg, 4.75 mol, 60-70% solution in toluene) was added slowly maintaining the temperature below −5 to 0° C. ° C. The reaction mixture was then warmed to 25° C. to 30° C. and stirred for not less than 12 hours until completion of reaction as confirmed by UPLC (disappearance of compound 1-2). In another reactor-2 10% NaOH solution (4.0 L) was cooled to 0° C. The reaction mixture was quenched by transferring into the cold 10% NaOH solution while maintaining the temperature below 30° C. After completion of transfer, quench mixture allowed to stir for 3 hours and then layers allowed to separate. The organic layer was separated. The organic layer was, washed with water (2.0 L), 15% brine (2.0 L), and evaporated to dryness. The crude residue of compound 2 (424 g), a pale yellow oil was used as is in next step.

Compound 2 (424 g, 1.7 mol) was taken in MeOH (1.7 L). Activated carbon (42.4 g, 0.10 w/w) added and heated to 40-45° C. for 2 h. The hot solution was filtered through hyflo bed and washed with MeOH (424 mL). Filtrate was transferred to hydrogenation autoclave flask, degassed and purged with N₂, twice. 10 wt % Pd/C (50% wet, 42.4 g) was charged and the mixture was degassed and purged with H₂, twice. The reaction mixture allowed to agitate under H₂ atmosphere (100 psi) for not less than 20 hours until completion of reaction as confirmed by UPLC. The mixture was degassed and purged with N₂, filtered through a pad of celite. The filtrate was evaporated to near dryness, co-distilled with Ethyl acetate (2×848 mL), triturated with Ethyl acetate (424 mL) at 25 to 30° C. for 3 h. Solids were filtered, washed with cold Ethyl acetate (212 mL) and dried in vacuum at <30° C. Compound 3 (180.0 g, 71%) was obtained as off-white solid. m/z 160.11 [M+H]⁺. ¹H NMR (400 MHz, DMSO-d₆) δ 4.18 (s, 2H), 3.34 (s, 2H), 3.25 (d, J=10.5 Hz, 2H), 2.80 (d, J=10.6 Hz, 2H), 2.44 (d, J=10.5 Hz, 2H), 0.88 (d, J=1.7 Hz, 6H).

### Example 2. Synthesis of Compound 4

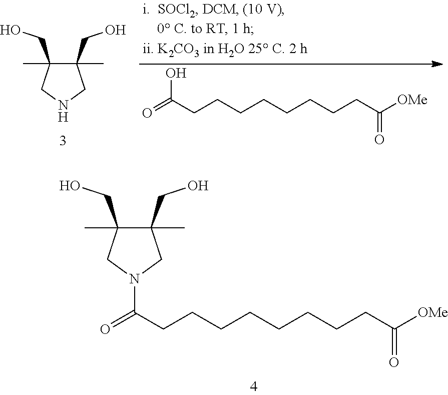 [SMILES: C[C@]1(CO)CNC[C@@]1(C)CO ; COC(=O)CCCCCCCCC(=O)O ; COC(=O)CCCCCCCCC(=O)N1C[C@](C)(CO)[C@](C)(CO)C1]

To a solution of methyl sebacate (135.0 g, 848 mmol) in DCM (1350 mL), thionyl chloride (100.9 g, 848 mmol) and DMF (1.0 g) were sequentially charged with agitation while maintaining internal temperature 20 to 30° C. The mixture allowed to agitate at 20° C. to 30° C. for not less than 2 hours until completion of reaction as confirmed by disappearance of methyl sebacate by UPLC. The mixture was evaporated to dryness and the residue co-distilled with DCM (675 mL) and then the acid chloride was made into a solution in DCM (675 mL). In another flask, a mixture of compound 3 in DCM (675 mL), water (1350 mL), K₂CO₃ (239.0 g, 2544 mmol) was cooled to 0 to 5° C. To this cold mixture, acid chloride solution in DCM was added slowly dropwise with agitation in four portions with 15 min intervals, maintaining the temperature below 5° C. The reaction mixture was then warmed to 25° C. to 30° C. and agitated for not less than 12 hours until completion of reaction as confirmed by UPLC. The mixture was diluted with Ethyl acetate (1500 mL), organic layer separated, washed with water (1350 mL), brine (675 mL), dried over Na₂SO₄ (135 g), evaporated and dried in vacuum at <45° C. Compound 4 (260 g, 86%) was obtained as light-yellow colored liquid. m/z 358.18 [M+H]⁺. ¹H NMR (400 MHz, DMSO-d₆) δ 4.66 (ddd, J=13.8, 6.1, 3.8 Hz, 2H), 3.51 (dd, J=10.2, 2.2 Hz, 1H), 3.41-3.32 (m, 1H), 3.31 (tt, J=7.6, 3.8 Hz, 4H), 3.09 (dd, J=10.3, 2.3 Hz, 1H), 2.99 (dd, J=11.9, 2.4 Hz, 1H), 2.49 (d, J=1.7 Hz, 1H), 2.27 (td, J=7.4, 2.4 Hz, 2H), 2.14 (t, J=7.6 Hz, 2H), 1.55-1.40 (m, 4H), 1.26-1.21 (m, 8H), 1.01-0.90 (m, 6H).

### Example 3. Synthesis of Compound 5

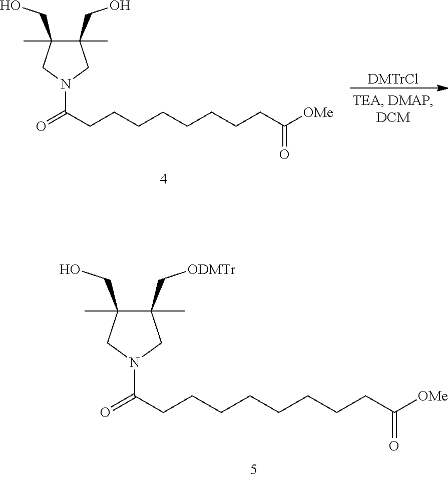

To a solution of compound 4 (35.0 g, 9.8 mmol) in DCM (350 mL), trimethylamine TEA (14.87 g, 14.7 mmol) and DMAP (1.2 g, 1.0 mmol) were added at 20° C. to 30° C. This mixture was cooled to 0° C. to 5° C. and DMTrCl (33.2 g, 9.8 mmol) was added. The reaction mixture allowed to stir at the same temperature for not less than 2 hours until completion of reaction as confirmed by UPLC. Water (350 mL) was added and mixture allowed to warm to 20 to 30° C. and stirred for 30 minutes. The aqueous layer was separated and extracted with DCM (70 mL). The organic layers were pooled and washed with aqueous NaHCO₃ solution (350 mL), brine (350 mL), dried over Na₂SO₄ (35.0 g), filtered and evaporated to dryness. The crude residue was purified by silica gel column chromatography (15 to 60% EA/hexanes) to give pure compound 5 (35.0 g, 54.1%) as pale-yellow oil. m/z 660.58 [M+H]⁺. ¹H NMR (400 MHz, Chloroform-d) δ 7.40 (d, J=8.1 Hz, 2H), 7.34-7.17 (m, 9H), 6.83 (dd, J=8.8, 2.6 Hz, 4H), 3.80 (s, 6H), 3.67 (s, 3H), 3.56 (d, J=12.3 Hz, 1H), 3.49-3.18 (m, 3H), 3.12 (d, J=9.9 Hz, 1H), 3.04 (s, 1H), 2.29 (td, J=7.7, 3.8 Hz, 2H), 2.17 (q, J=6.2, 4.8 Hz, 2H), 2.08-2.02 (m, 1H), 1.30 (s, 12H), 1.18 (d, J=23.3 Hz, 3H), 1.03 (d, J=4.8 Hz, 3H).

### Example 4. Synthesis of Compound 6

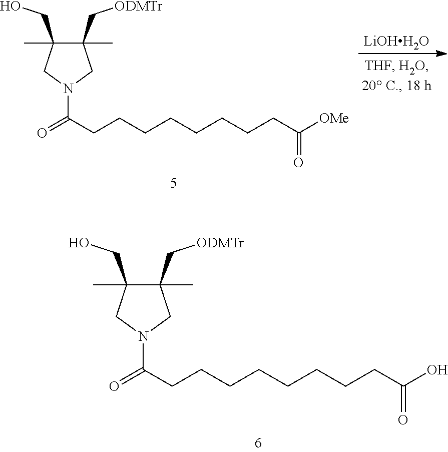

A solution of compound 5 (35.0 g, 5.3 mmol) in MeOH/Water (1:1, 700 mL) was cooled to 0° C. LiOH.H₂O (4.86 g, 11.6 mmol) was added and mixture stirred for not less than 1 hour until completion of reaction as confirmed by UPLC. Methanol was evaporated, water added and the mixture cooled to 0 to 5° C. The mixture was neutralized to −pH 7.0 with sodium dihydrogen phosphate solution and then acidified to pH 6 to 6.5 using acetic acid while maintaining the temperature below 5° C. The aqueous mixture was extracted with DCM (2×350 mL) and evaporated to complete dryness and then further dried in a vacuum oven at 45° C. Compound 6 (28.3 g, 82%) was obtained as off-white solid. m/z 646.54 [M+H]⁺. ¹H NMR (400 MHz, DMSO-d₆) δ 11.96 (s, 1H), 7.32 (p, J=7.6 Hz, 4H), 7.21 (t, J=7.6 Hz, 5H), 6.87 (d, J=8.2 Hz, 4H), 4.60-4.48 (m, 1H), 3.72 (s, 6H), 3.46 (dd, J=30.2, 11.0 Hz, 1H), 3.20 (dd, J=25.3, 11.0 Hz, 1H), 3.13-2.83 (m, 5H), 2.12 (dq, J=31.7, 7.6 Hz, 4H), 1.49-1.41 (m, 4H), 1.22 (d, J=10.2 Hz, 10H), 1.11-0.99 (m, 4H).

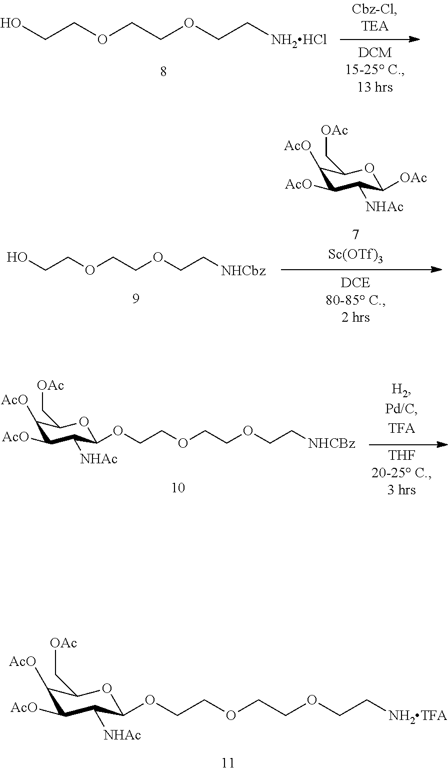

### Example 5. Synthesis of Compound 9

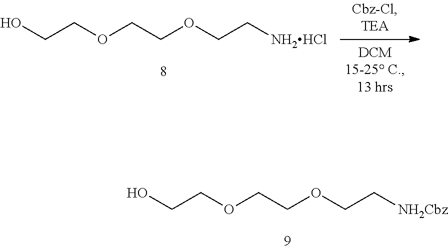

19.0 g (1.0 equiv) of Compound 8 after azeotrope concentration with toluene to remove any water content) and 190 mL (10 V) of DCM were charged to a 500 mL of reactor. After cooling to −20° C., 20.6 g (0.95 equiv) of Cbz-Cl was slowly charged at −20˜−10° C. for 2 hours using a syringe pump. Then, 14.2 g (1.10 equiv) of TEA was slowly charged at −20˜−7° C. for 2 hours using a syringe pump. The reaction mixture was agitated at room temperature for 17 hours to complete the reaction conversion. The contents were washed with 95 mL (5 V) of 1N HCl, 95 mL (5 V) of 8 wt % NaHCO₃ and 95 mL (5 V) of brine in a sequence. Then, 38 mL (2 V) of purified water was added to the organic layer and the contents were concentrated under 20 torr at 65° C. with a water bath. The process for azeotrope concentration was repeated 2 times with water (distillation with water removes impurities). After azeotrope concentration, the concentrate was diluted with 57 mL (3 V) of DCM and treated with 19 g (1 S) of Na₂SO₄. The contents were filtered and the waste was washed with 38 mL (2 V) of DCM. The filtrates were concentrated under 20 torr at 65° C. with a water bath, and then it was dried under full vacuum at 50° C. for weekend to give the product compound 9 (185 g, 97% Yield) as a colorless oil. m/z 284.2 [M+H]⁺. ¹H NMR (600 MHz, DMSO-d₆) δ 7.39-7.28 (m, 3H), 7.32-7.22 (m, 2H), 5.73 (s, 1H), 5.00 (s, 3H), 4.56 (t, J=5.5 Hz, 2H), 3.47 (t, J=5.3 Hz, 4H), 3.40 (td, J=5.6, 5.2, 2.2 Hz, 7H), 3.33 (s, 1H), 3.14 (q, J=6.0 Hz, 4H)

### Example 6. Synthesis of Compound 10

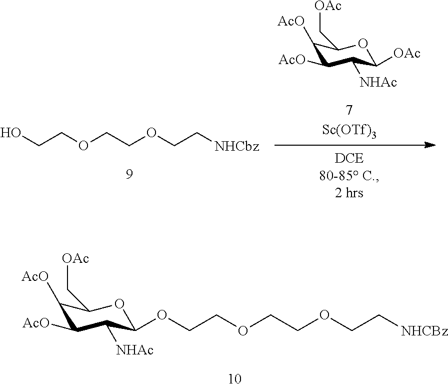

To a solution of compound 9 (10 g, 35.3 mmol) in DCE (100 mL) was added compound 7 (16.5 g, 42.4 mmol) and TMSOTf (0.6 mL, 3.5 mmol). The mixture was agitated at 60 to 65° C. for not less than 3 hours until completion of reaction as confirmed by UPLC. The mixture was allowed to cool to 20° C. to 25° C. and sequentially washed with 8 wt % aq. NaHCO₃ (2×60 mL), 1N HCl (120 mL), brine (120 mL), dried over Na₂SO₄ (120 g) and evaporated to dryness to give compound 10 (22.7 g, quantitative yield) as a light-yellow syrup. m/z 613.3 [M+H]⁺. ¹H NMR (600 MHz, DMSO-d₆) δ 7.78 (d, J=9.2 Hz, 1H), 7.38-7.26 (m, 5H), 7.29-7.22 (m, 1H), 5.20 (d, J=3.4 Hz, 1H), 5.01-4.93 (m, 3H), 4.54 (d, J=8.5 Hz, 1H), 4.01 (m, 3H), 3.86 (m, 1H), 3.76 (m, 1H), 3.60-3.51 (m, 1H), 3.54-3.43 (m, 6H), 3.39 (t, J=6.0 Hz, 2H), 3.13 (q, J=6.0 Hz, 2H), 2.08 (s, 3H), 1.98 (s, 3H), 1.87 (s, 3H), 1.75 (s, 3H).

### Example 7. Synthesis of Compound 11

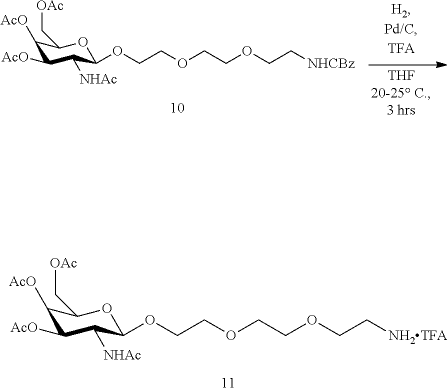

To a solution of compound 10 (110 g, 179 mmol) in THF (100 mL), TFA (20.5 g, 179 mmol) was added. The mixture was degassed and purged with N₂, twice. 10 wt % Pd/C (11 g) was charged and the mixture was degassed and purged with H₂, twice. The mixture was allowed to agitate under H₂ atmosphere (70 psi) for not less than 3 hours until completion of reaction as confirmed by UPLC. The mixture was degassed and purged with N₂, filtered through a pad of celite. The filtrate was evaporated to complete dryness to give compound 11 (106 g, quantitative yield) as a light-yellow foamy solid. m/z 479.2 [M+H]⁺. ¹H NMR (600 MHz, DMSO-d₆) δ 7.93 (dd, J=12.4, 5.3 Hz, 4H), 5.20 (d, J=3.4 Hz, 1H), 4.96 (dd, J=11.2, 3.4 Hz, 1H), 4.54 (d, J=8.5 Hz, 1H), 4.06-3.96 (m, 3H), 3.88 (dt, J=11.1, 8.8 Hz, 1H), 3.78 (m, 1H), 3.58 (t, J=5.2 Hz, 3H), 3.58-3.45 (m, 6H), 2.96 (h, J=5.6 Hz, 2H), 2.08 (s, 3H), 1.98 (s, 3H), 1.87 (s, 3H), 1.76 (s, 3H).

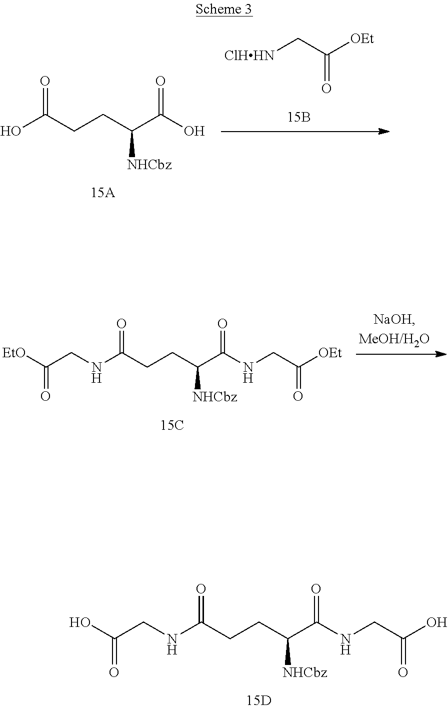

### Example 8. Synthesis of Compound 15C

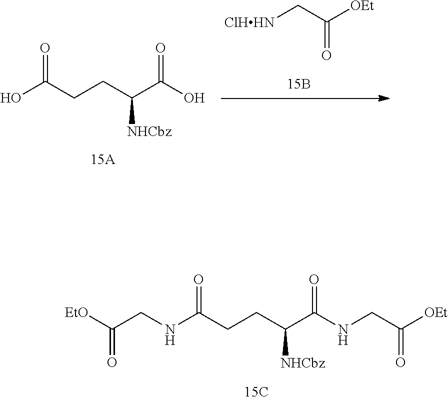

A solution of DMF (1000 mL) and DIPEA (275.3 g, 2.13 mmol) was cooled to 0 to 5° C. Sequentially charged compound 15-A (100 g, 0.35 mol), EDC.HCl (217.1 g, 1.13 mol) and HOBt monohydrate (173.9 g, 1.13 mol) while maintaining temperature 0 to 5° C. Agitated for 10 min and then compound 15-B (163.4 g, 1.17 mol) was charged. The reaction mixture was allowed to warm to 25° C. to 30° C. and stirred for not less than 16 hours until completion of reaction as confirmed by UPLC. The reaction mixture was diluted by slow addition of Ethanol (1000 mL) followed by water (4500 mL) and allowed to stir for not less than 4 h at 25° C. to 30° C. A precipitate formed which was filtered, washed with water (1000 mL) and solids dried in vacuum at <50° C. Compound 15C (134.0 g, 83% Yield) was obtained as a white solid. m/z 452.21 [M+H]⁺.

### Example 9. Synthesis of Compound 15D

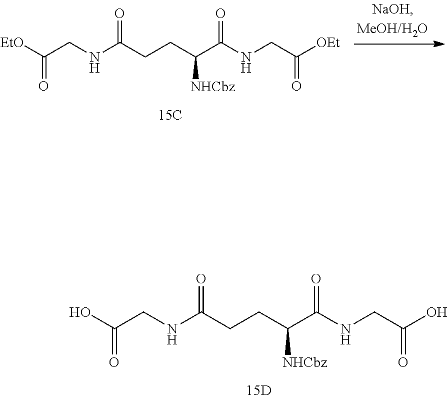 [SMILES: CCOC(=O)CNC(=O)CC[C@H](NC(=O)OCc1ccccc1)C(=O)NCC(=O)OCC ; O=C(O)CNC(=O)CC[C@H](NC(=O)OCc1ccccc1)C(=O)NCC(=O)O]

To a solution of compound 15C (50 g, 11.1 mmol) in MeOH/Water (1:1, 500 mL), a solution of NaOH (9.8 g, 24.4 mmol) in water (250 mL) was added slowly at 25° C. to 35° C. The reaction mixture was allowed to stir for not less than 6 hours until completion of reaction as confirmed by UPLC. MeOH was evaporated. The aqueous solution was rendered acidic to pH-1-2 by addition of 6.0 N HCl solution and saturated with NaCl. The aqueous layer was extracted with ethyl acetate (3×750 mL). The ethyl acetate layers were pooled, dried over Na—SO₄ (100 g) and evaporated to dryness. The crude residue was triturated with hexanes (250 mL), filtered, washed with hexanes (100 mL) and dried under vacuum at 45° C. Compound 15D (20.0 g, 49%) was obtained as a white solid. m/z 396.11 [M+H]t

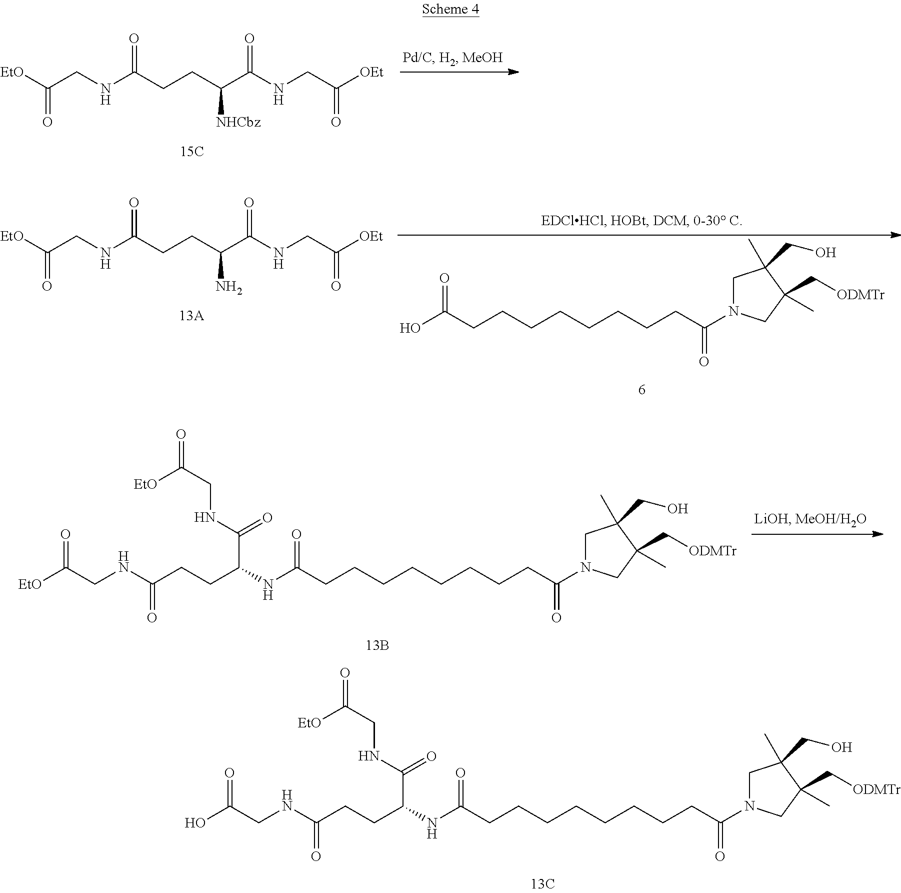

### Example 10. Synthesis of Compound 13A

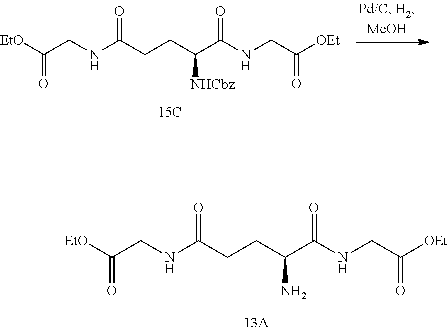 [SMILES: CCOC(=O)CNC(=O)CC[C@H](NC(=O)OCc1ccccc1)C(=O)NCC(=O)OCC ; CCOC(=O)CNC(=O)CC[C@H](N)C(=O)NCC(=O)OCC]

A solution of compound 15C (80.0 g, 0.17 mol) in THF(2800 mL) was degassed and purged with N₂, twice. 10 wt % Pd/C (50% wet, 8.0 g) was charged and the mixture was degassed and purged with H₂, twice. The mixture was allowed to agitate under H₂ atmosphere (100 psi) for not less than 6 hours until completion of reaction as confirmed by UPLC. The mixture was degassed and purged with N₂, filtered through a pad of celite. The filtrate was evaporated, solvent swapped with Ethyl acetate (2×400 mL). Residue taken in Ethyl acetate (400 mL) at 45° C. and n-Heptane (320 mL) slowly added, stirred at 45° C. for 1 h and then at 0 to 5° C. for 1 h. Solids were filtered, washed with cold solution of Ethyl acetate/n-Heptane (1:2, 160 mL) and dried in vacuum oven 25 to 30° C. to give compound 13A (46.2 g, 82% yield) as white solid. m/z 318.14 [M+H]⁺.

### Example 11. Synthesis of Compound 13B

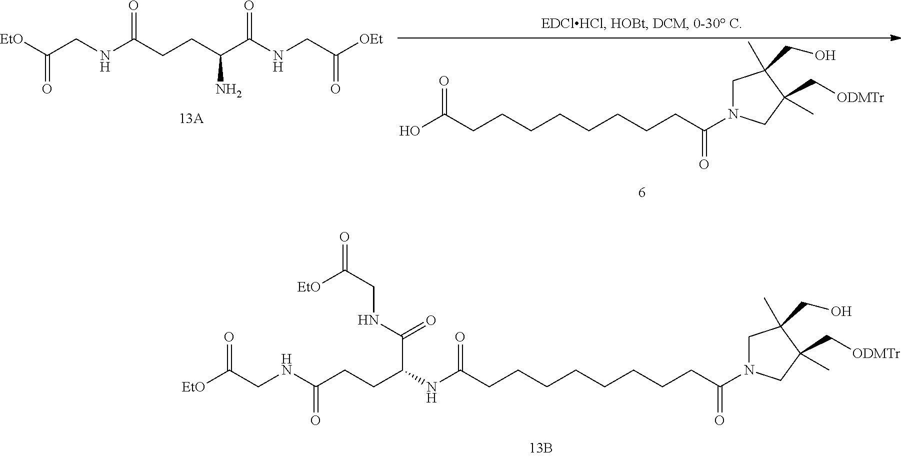

A solution of DCM and DIPEA (5.9 g, 46.4 mmol) was cooled to 0 to 5° C. Sequentially charged compound 6 (15.0 g, 23.2 mmol), EDC.HCl (5.1 g, 26.7 mmol), HOBt monohydrate (4.1 g, 26.7 mmol) while maintaining 0 to 5° C. Mixture was stirred for 10 min, compound 13A (7.74 g, 24.3 mmol) was charged and allowed to stir at 0 to 5° C. for NLT 21 h until completion of reaction as confirmed by UPLC. Charged purified water (150 mL) while maintaining below 30° C. Organic layer separated, washed with aq. NaHCO₃ (2×105 mL) and 10% aq. NaCl. Organic layer evaporated, solvent swapped with Ethyl acetate twice (300 ml and 150 mL). When at 4V total volume, n-Heptane added and heated 50 to 55° C. for 1 h and then cooled at 0 to 5° C. for 1 h. Solids filtered, washed with mother liquor, n-Heptane (30 mL) an dried solids in vacuum at 40 to 45° C. Compound 13B (17.9 g, 81%) was obtained as white solid. m/z 946.05 [M+H]t

### Example 12. Synthesis of Compound 13C

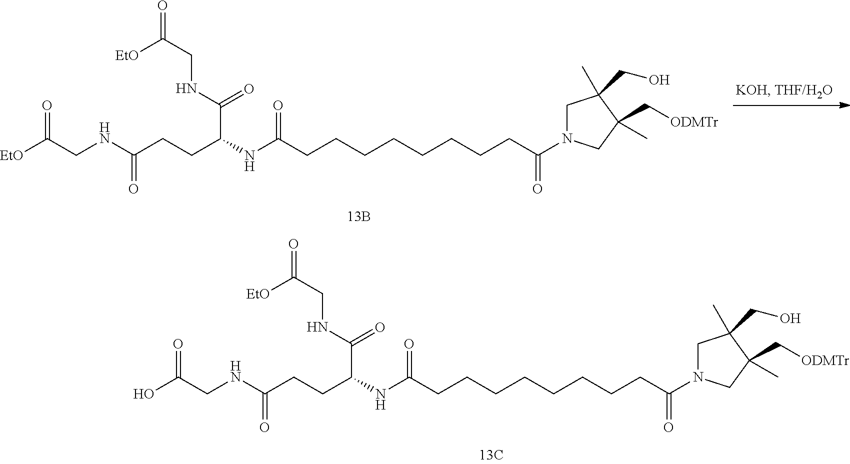

A solution of compound 13B (10.0 g, 10.6 mmol) in THF (100 mL) was cooled to 0 to 5° C. KOH solution (1.5 g, 26.4 mmol in 50 mL water) was added at below 5° C. The reaction mixture was warmed to 25 to 30° C. and stirred for not less than 4 hours until completion of reaction as confirmed by UPLC. Mixture cooled to 0 to 5° C., adjusted pH to ˜7.0 with aqs. Sodium dihydrogen phosphate solution while maintaining temperature below 10° C. Charged 2-methyltetrahydrofuran (150 mL). Adjusted pH to 4-5 by using 1N HCl while maintaining temperature below 10° C. Allowed the mixture to stir at 25 to 30° C. for 20 min. Separated organic layer, back washed aqueous layer with 2-methyltetrahydrafuran. Organic layers pooled and washed with 10% aqs NaCl (50 mL). To organic layer triethylamine (4.3 g, 42.4 mmol) was added, allowed to stir for 1 h and then evaporated, solvent swapped with tetrahydrofuran (20 mL) and then evaporated to complete dryness and further dried in vacuum oven. compound 13C (TEA salt) (9.0 g, 84%) was obtained as a white hygroscopic solid. m/z 889.13 [M+H]t

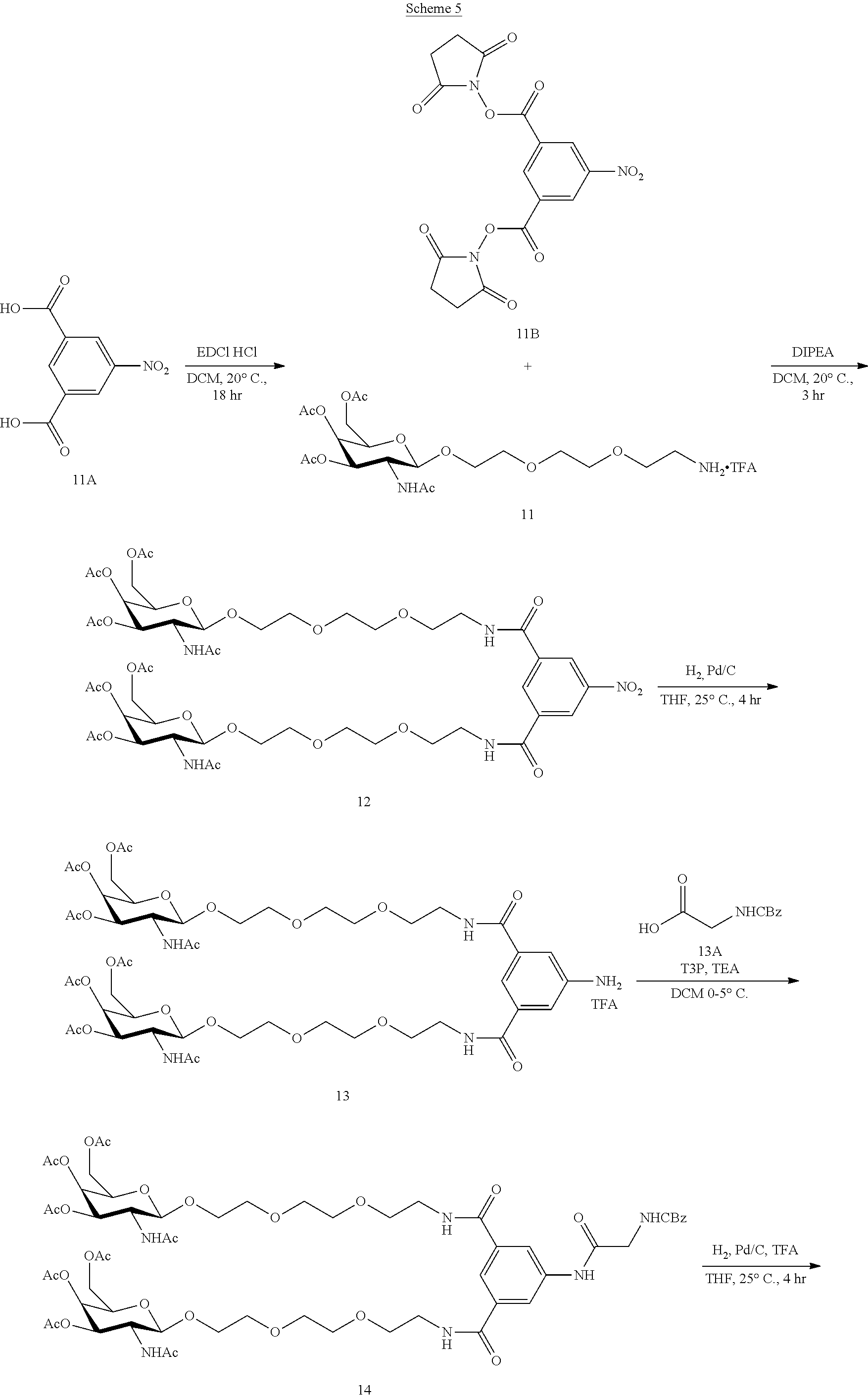

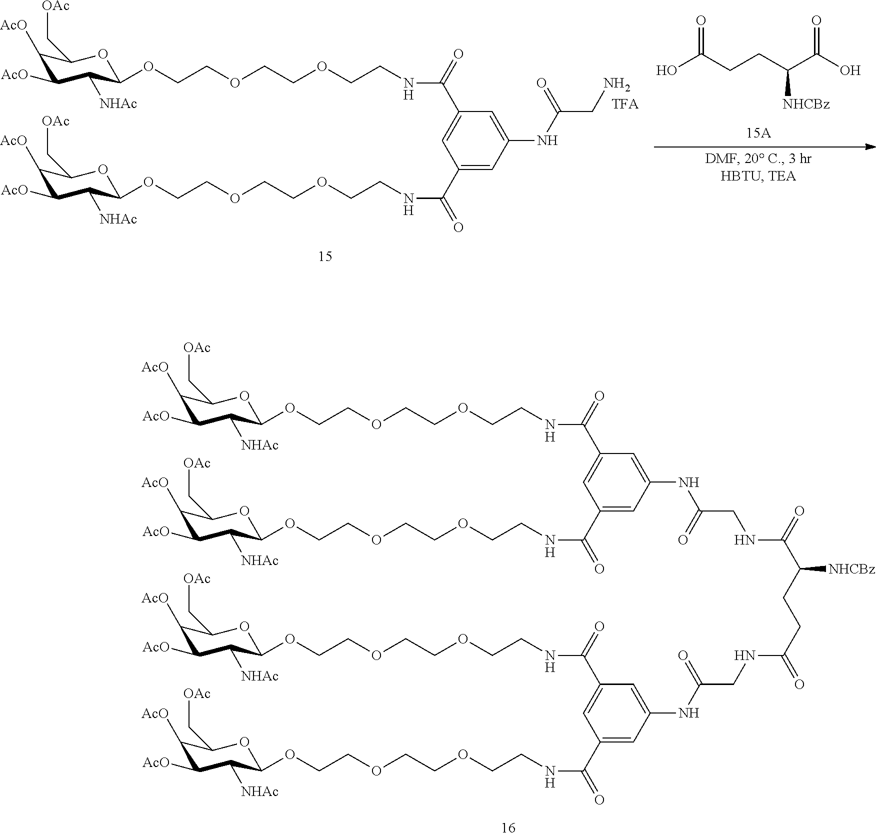

### Example 13. Synthesis of Compound 12

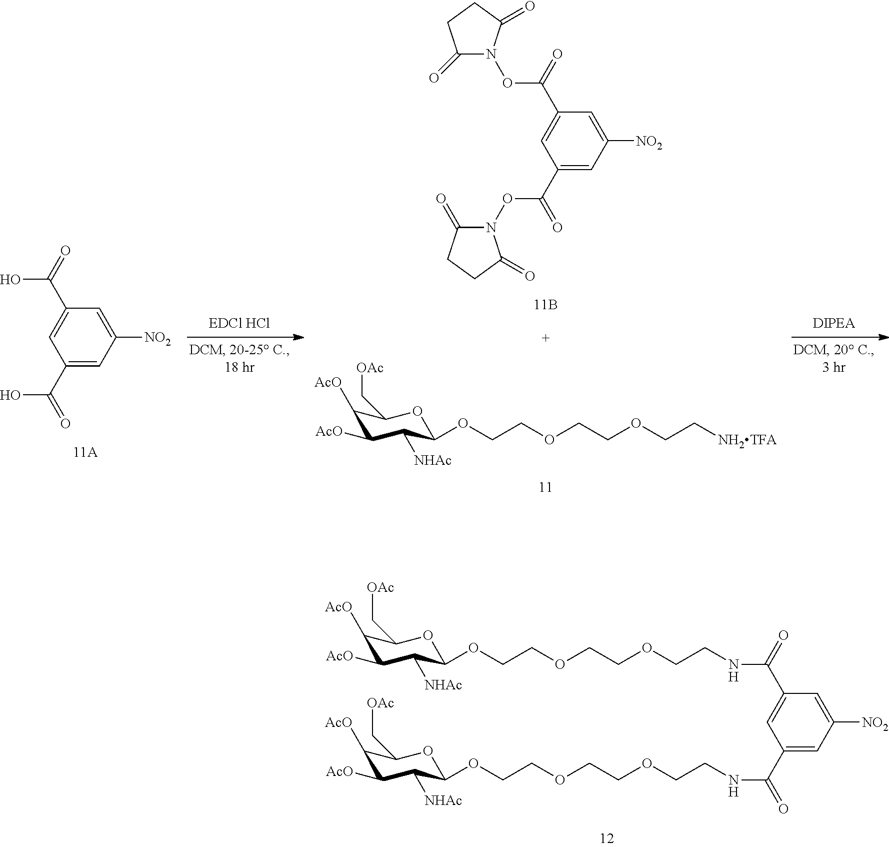

To a solution of compound 16-1 (41.5 g, 196.6 mmol) in DCM (415 mL), N-Hydroxysuccinimide (49.7 g, 432.4 mmol) and EDC.HCl (82.9 g, 432.4 mmol) were added. The reaction mixture was allowed to stir at 25° C. to 30° C. for not less than 16 hours until completion of reaction to form compound 11B as confirmed by TLC. Reaction mixture was evaporated to 2-3V, water (415 mL) was added and allowed to stir the solids at 25 to 30° C. for 1 h. Filtered the solids, washed with water (415 mL) and wet solids triturated with aqs. NaHCO₃ solution (415 mL) at 25 to 30° C. for 1 h. Solids were filtered again, washed with water (415 mL), MTBE (210 mL) and dried in vacuum below 45° C. to give compound 11B as a white solid (45.0 g, 56% yield).

A solution of compound 11 (110.0 g, 185.64 mmol) in DCM (1100 mL) was cooled to 0 to 5° C. Compound 11B (33.85 g, 83.54 mmol) and DIPEA (47.9 g, 371.3 mmol) were charged at below 10° C. and allowed to stir at 20° C. to 25° C. for no less than 3 hours until completion of reaction as confirmed by UPLC. Water (1100 mL) was added to the mixture at below 30° C. and stirred for 45 min. Organic layer was separated, washed with aqs. NaHCO₃ solution (1100 mL), 1N HCl (1100 mL) and 15% aqs. NaCl (1100 mL). Organic layer evaporated and solvent swapped with MTBE (500 mL) and taken into MTBE (500 mL), stirred for 3 h at 25 to 30° C. Resulting solids were filtered, washed with MTBE (250 mL) and dried in vacuum at <50° C. Compound 12 (90.0 g, 86% Yield) was obtained as a light-yellow foam solid. m/z 1132.5 [M+H]⁺.

### Example 14. Synthesis of Compound 13

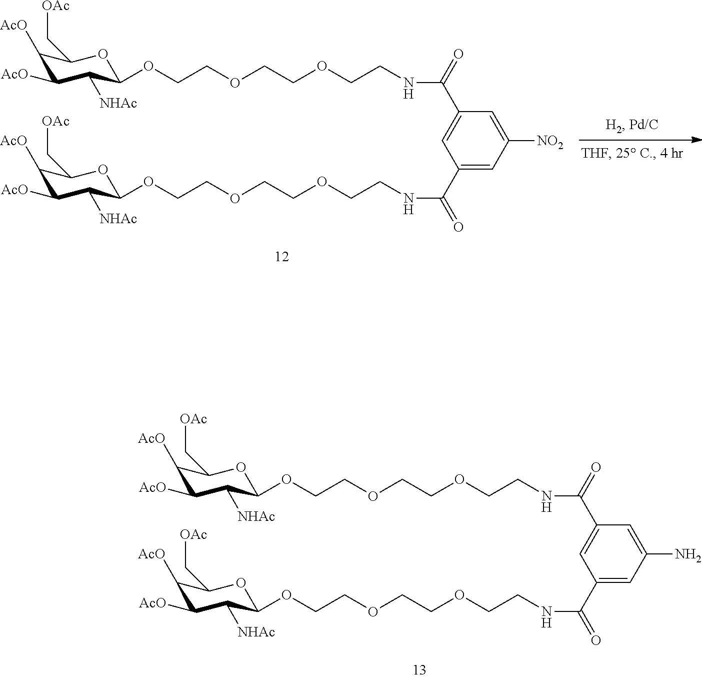

A solution of compound 12 (39 g, 34.45 mmol) in THF (240 mL) was degassed and purged with N₂, twice. 10 wt % Pd/C (3.9 g) was charged and the mixture was degassed and purged with H₂, twice. The reaction mixture allowed to agitate under H₂ atmosphere for not less than 4 hours until completion of reaction as confirmed by UPLC. The mixture was degassed and purged with N₂, filtered through a pad of celite (39 g). The filtrate was evaporated to complete dryness to give compound 13 (36.4 g, 95% yield) as a grey foam solid. m/z 1102.5 [M+H]⁺.

### Example 15. Synthesis of Compound 14

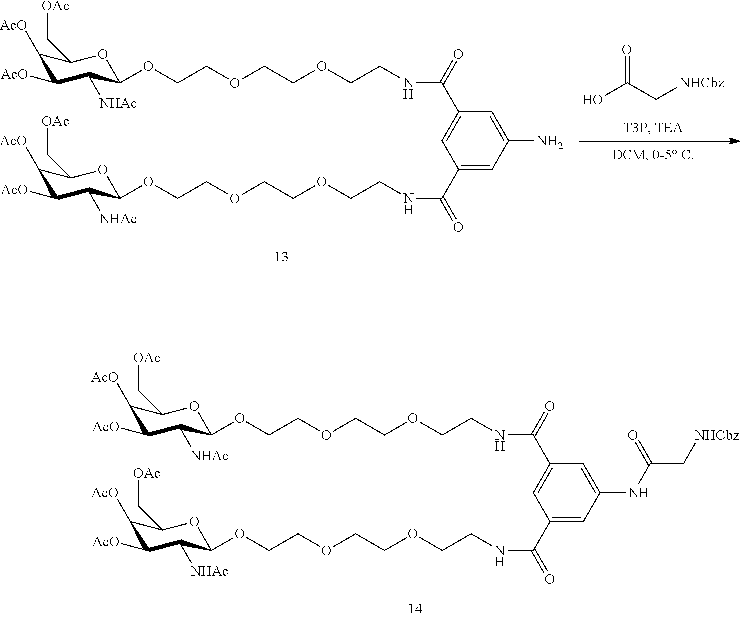

A solution of compound 13 (30 g, 22.72 mmol) and N-carbobenzoxyglycine (7.97 g, 38.11 mmol), in DCM (150 mL) was cooled to 0 to 5° C., the following were charged sequentially with agitation at below 5° C., TEA (7.6 mL, 54.44 mmol), and T3P (29.2 mL, 49 mmol, 50% solution in ethyl acetate). The reaction mixture was agitated at 0° C. to 5° C. for not less than 3 hours until completion of reaction as confirmed by UPLC. Reaction mixture was washed sequentially with water (110 mL), saturated aq. NaHCO₃ (110 mL), brine (110 mL), then dried over Na₂SO₄ (60 g) and evaporated to complete dryness to give compound 14 (32 g, 91% yield) as a grey foam solid. m/z 1293.6 [M+H]t

### Example 16. Synthesis of Compound 15

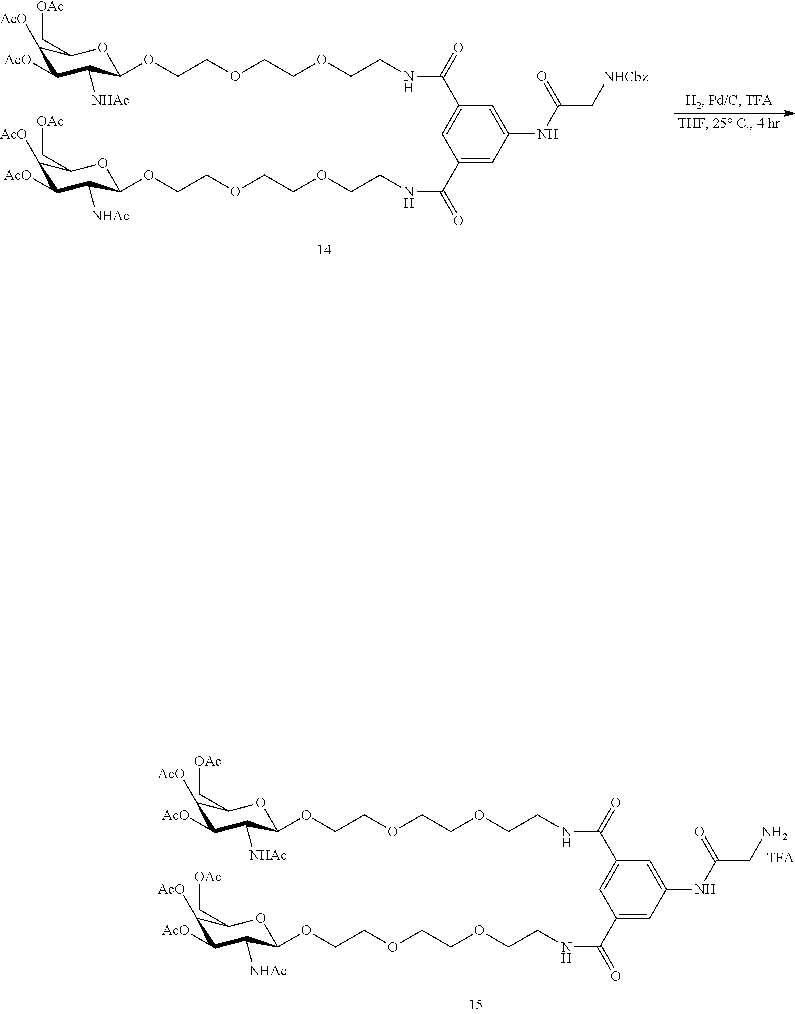

A solution of compound 14 (68 g, 52.78 mmol) in THF (400 mL) was degassed and purged with N₂, twice. 10 wt % Pd/C (6.8 g) and TFA (4.4 mL, 57.84 mmol) were charged and the mixture was degassed and purged with H₂, twice. The reaction mixture allowed to agitate under H₂ atmosphere for not less than 4 hours until completion of reaction as confirmed by UPLC. The mixture was degassed and purged with N₂, filtered through a pad of celite (68 g). The filtrate was evaporated to complete dryness to give compound 15 (63 g, 95% yield) as a grey foam solid. m/z 1159.6 [M+H]⁺ (Free base).

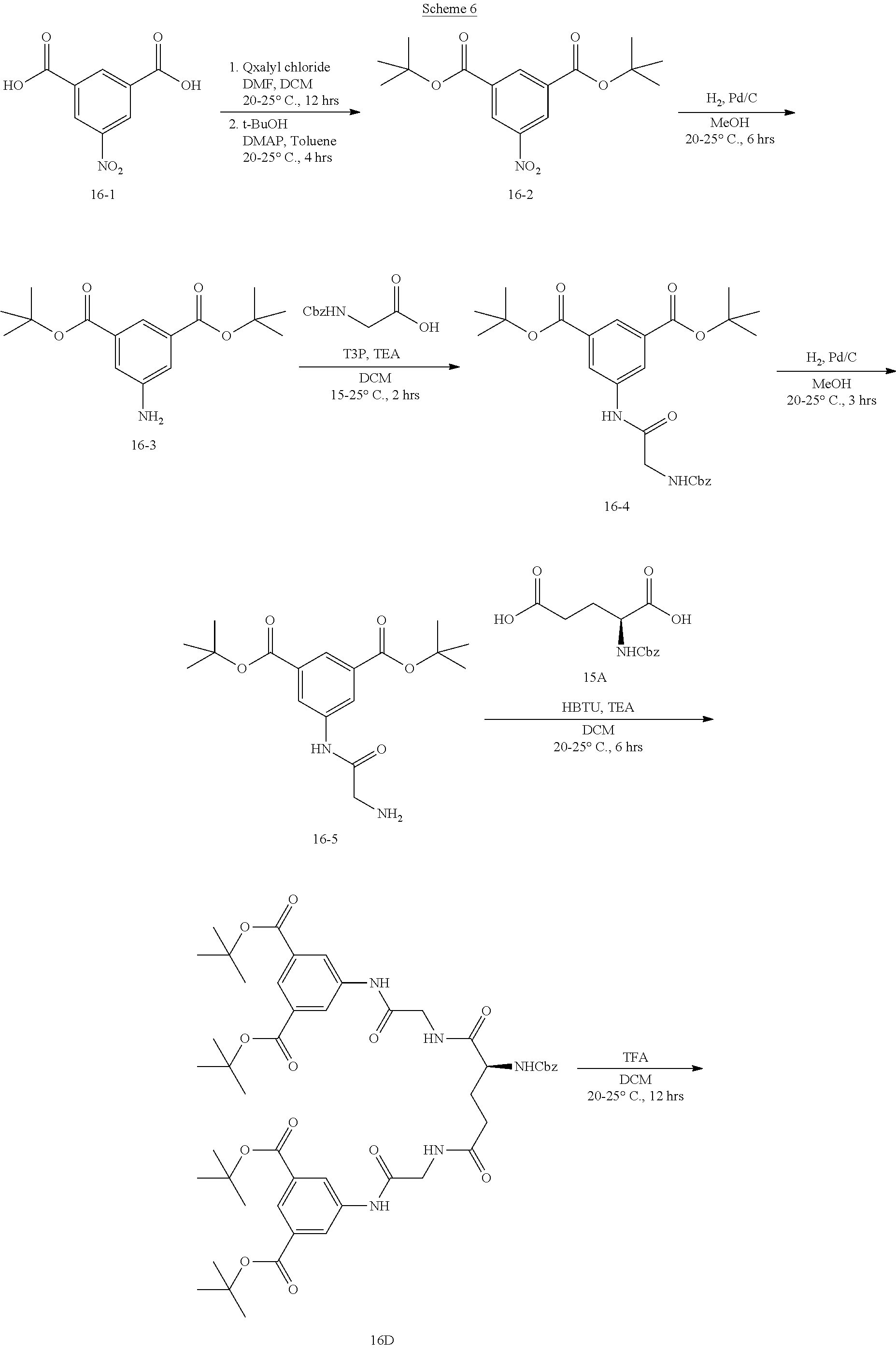

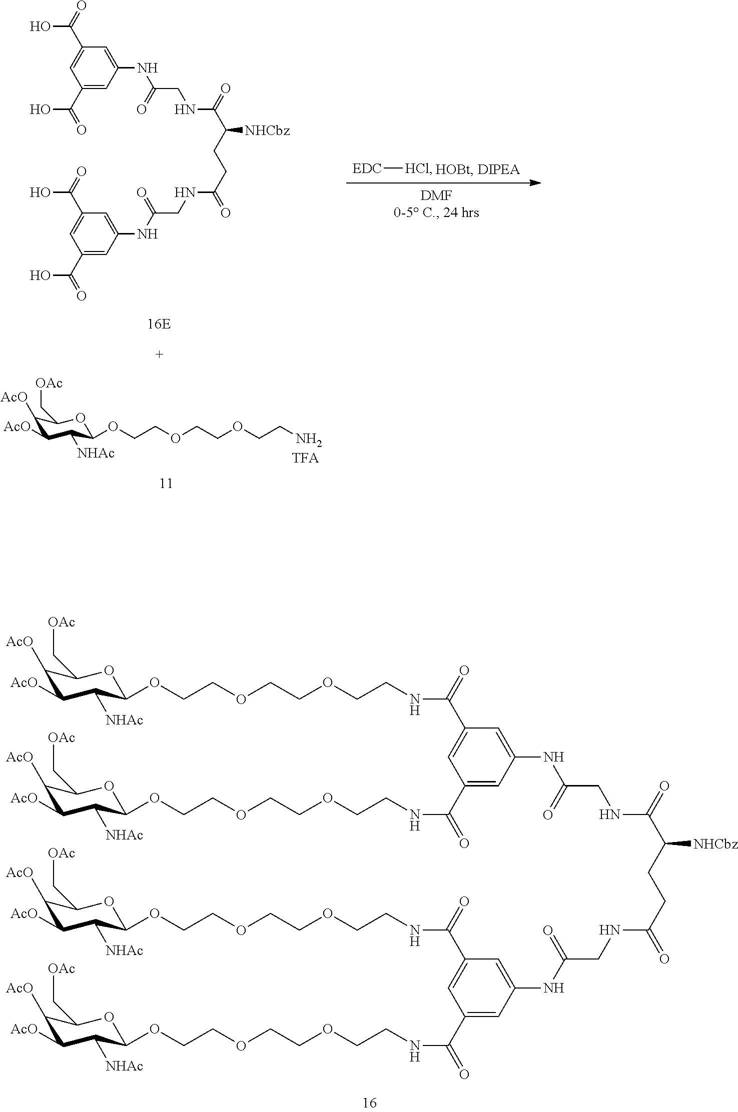

### Example 17. Synthesis of Compound 16-2

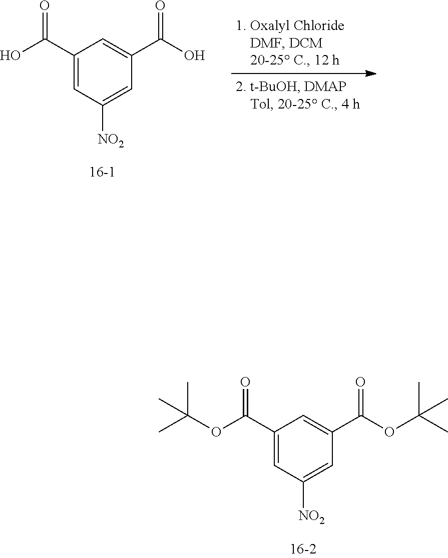 [SMILES: O=C(O)c1cc(C(=O)O)cc([N+](=O)[O-])c1 ; CC(C)(C)OC(=O)c1cc(C(=O)OC(C)(C)C)cc([N+](=O)[O-])c1]

To a suspension of compound 16-1 (50 g, 236 mmol) in DCM (500 mL), oxalyl chloride (69 g, 543 mmol) and DMF (172 mg, 2.3 mmol) were sequentially charged with agitation while maintaining internal temperature 20 to 30° C. The mixture was allowed to agitate 20° C. to 30° C. for not less than 12 hours until completion of reaction as confirmed by disappearance of compound 16-1 by UPLC. The mixture is evaporated to dryness and the residue is taken into toluene. To the toluene solution were charged t-BuOH (52.5 g, 708 mmol) and DMAP (66.3 g, 543 mmol). This mixture was allowed to agitate for not less than 4 hours at 20° C. to 25° C. until completion of reaction as confirmed by UPLC. The mixture was filtered, filtrate washed with 5% aqueous citric acid solution (500 mL), brine (500 mL), dried over NaSO₄ (100 g) and evaporated. The residue was azeotroped with n-hexane (250 mL), evaporated and dried to give compound 16-2 (73 g, 95% Yield) as off-white solid. m/z 341.2 [M+H]t ¹H NMR (400 MHz, Chloroform-d) δ 8.92 (s, 2H), 8.87 (s, 1H), 1.64 (s, 18H).

### Example 18. Synthesis of Compound 16-3

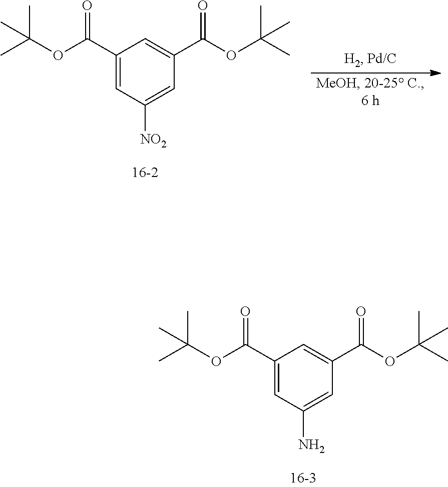

A solution of compound 16-2 (30 g, 92.3 mmol) in MeOH (360 mL) was degassed and purged with N₂, twice. 10 wt % Pd/C (3 g) was charged and the mixture was degassed and purged with H₂, twice. The mixture was allowed to agitate under H₂ atmosphere for not less than 6 hours until completion of reaction as confirmed by UPLC. The mixture was degassed and purged with N₂, filtered through a pad of celite. The filtrate was evaporated to complete dryness to give compound 16-3 (26 g, 95% yield) as off-white solid. m/z 294.2 [M+H]t ¹H NMR (400 MHz, Chloroform-d) δ 7.96 (d, J=1.7 Hz, 1H), 7.45 (d, J=1.5 Hz, 2H), 3.92 (s, 2H), 1.59 (s, 18H).

### Example 19. Synthesis of Compound 16-4

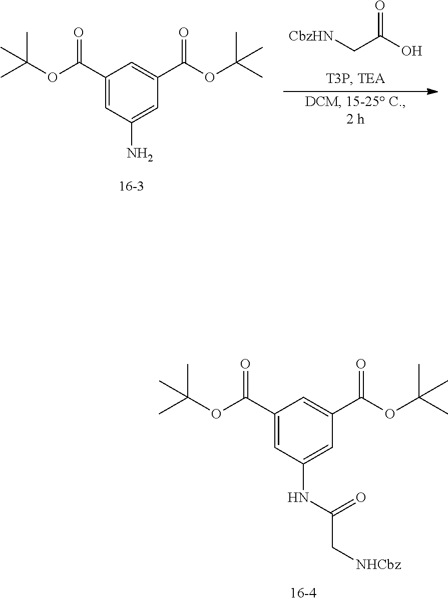

To a solution of compound 16-3 (23.7 g, 80.7 mmol) in DCM (355 mL), the following were charged sequentially with agitation at 15 to 25° C., N-carbobenzoxyglycine (23.7 g, 113 mmol), TEA (16.3 g, 161 mmol), T3P (92.3 g, 145 mmol, 50% solution in ethyl acetate). The mixture was agitated at 15° C. to 25° C. for not less than 2 hours until completion of reaction as confirmed by UPLC. The mixture was washed sequentially with water (240 mL), saturated aq.NaHCO₃ (240 mL), brine (240 mL), then dried over Na₂SO₄ (48 g) and evaporated to complete dryness to give compound 16-4 (46.2 g, 118% yield) as a pale yellow solid. m/z 484.2 [M+H]⁺. ¹H NMR (400 MHz, Chloroform-d) δ 8.95 (s, 1H), 8.34 (s, 2H), 8.29 (s, 1H), 7.36-7.27 (m, 5H), 5.99 (s, 1H), 5.16 (s, 2H), 4.12 (d, J=5.6 Hz, 2H), 1.57 (s, 18H).

### Example 20. Synthesis of Compound 16-5

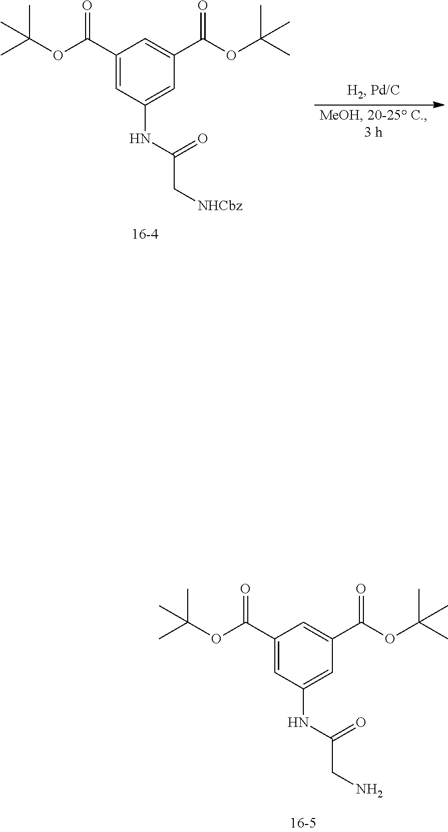

A solution of compound 16-4 (46.2 g, 95.3 mmol) in MeOH (1150 mL) was degassed and purged with N₂, twice. 10 wt % Pd/C (4.6 g) was charged and the mixture was degassed and purged with H₂, twice. The mixture was allowed to agitate under H₂ atmosphere for not less than 3 hours until completion of reaction as confirmed by UPLC. The mixture was degassed and purged with N₂, filtered through a pad of celite. The filtrate was evaporated, residue taken in methylene chloride (500 mL), evaporated to complete dryness to give compound 16-5 (32.4 g, 97% yield) as pale yellow solid. m/z 351.2 [M+H]⁺. ¹H NMR (600 MHz, Chloroform-d) δ 9.63 (s, 1H), 8.37 (d, J=1.6 Hz, 2H), 8.31 (t, J=1.5 Hz, 1H), 3.49 (d, J=18.7 Hz, 4H), 1.59 (s, 18H).

### Example 21. Synthesis of Compound 16D

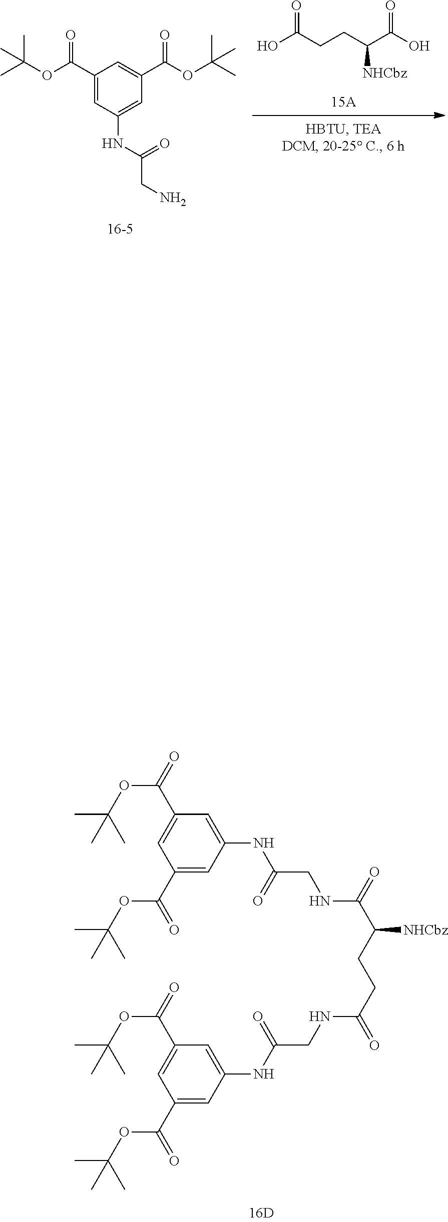

To a solution of compound 16-5 (32.5 g, 92.7 mmol) in methylene chloride (455 mL), compound 15A (11.7 g, 41.7 mmol) and HBTU (52.7 g, 139 mmol) were added. To this mixture TEA (28.1 g, 278 mmol) was added while maintaining internal temperature at 15° C. to 25° C. Reaction mixture allowed to stir at the same temperature for not less than 6 hours until completion of reaction as confirmed by UPLC. Reaction mixture was sequentially washed with water (320 mL), aqs. NaHCO₃ (320 mL), brine (320 mL), dried over Na₂SO₄ and evaporated to dryness. The crude residue was purified by column chromatography (30% to 100% EA/Hexanes) to give compound 16D (45.2 g, 51% Yield) as a white solid. m/z 946.5 [M+H]t ¹H NMR (600 MHz, DMSO-d₆) δ 8.39 (d, J=15.5 Hz, 5H), 8.22 (t, J=5.7 Hz, 1H), 8.05 (s, 2H), 7.35-7.24 (m, 5H), 5.06 (t, J=9.3 Hz, 2H), 4.05 (q, J=7.1 Hz, 1H), 3.98-3.87 (m, 4H), 2.28 (hept, J=7.9, 7.1 Hz, 2H), 1.96 (dt, J=18.0, 6.7 Hz, 1H), 1.82 (dd, J=14.5, 7.2 Hz, 1H), 1.53 (d, J=3.5 Hz, 36H).

### Example 22. Synthesis of Compound 16E

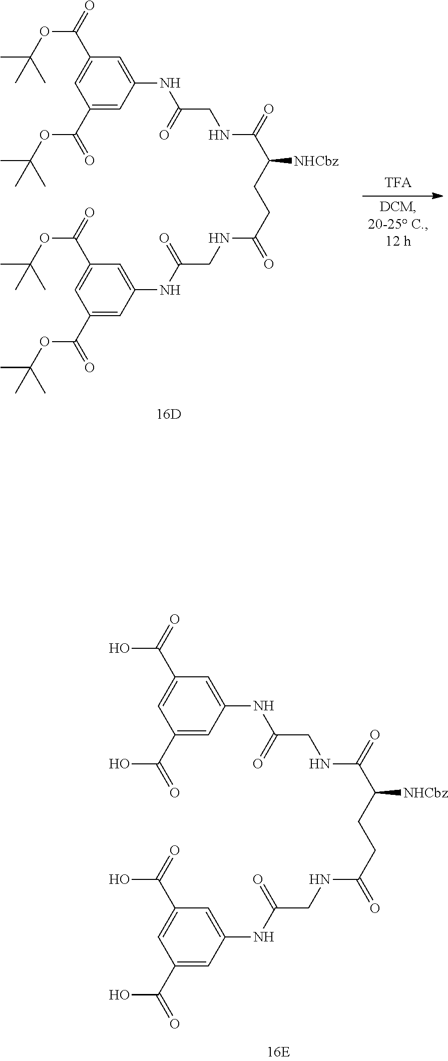 [SMILES: CC(C)(C)OC(=O)c1cc(NC(=O)CNC(=O)CC[C@H](NC(=O)OCc2ccccc2)C(=O)NCC(=O)Nc2cc(C(=O)OC(C)(C)C)cc(C(=O)OC(C)(C)C)c2)cc(C(=O)OC(C)(C)C)c1 ; "DCM,20-25° C.,12 h" ; O=C(CC[C@H](NC(=O)OCc1ccccc1)C(=O)NCC(=O)Nc1cc(C(=O)O)cc(C(=O)O)c1)NCC(=O)Nc1cc(C(=O)O)cc(C(=O)O)c1 ; "TFA"]

To a solution of compound 16D (30.0 g, 31.7 mmol) in methylene chloride (600 mL), TFA (108.4 g, 951 mmol) was added while maintaining internal temperature at 15° C. to 25° C. Reaction mixture was allowed to stir at 20° C. to 25° C. for not less than 12 hours until completion of reaction as confirmed by ¹H NMR. Mixture was evaporated to dryness, residue taken in methylene chloride (300 mL) and evaporated again to dryness. The resulting residue was partitioned between methylene chloride (300 mL) and 8 wt % aqs. NaHCO₃ solution (600 mL). Organic layer separated, aqueous layer washed again with methylene chloride (300 mL). Methylene chloride layers discarded. Aqueous layer acidified with 3N HCl (˜600 mL) to adjust to pH 3-4. The solids formed were filtered, washed with water and dried at 45° C. for not less than 12 hours to give compound 16E (16.4 g, 95% yield) as a white solid. m/z 722.2 [M+H]⁺. ¹H NMR (600 MHz, DMSO-d₆) δ 10.31 (s, 1H), 10.11 (s, 1H), 8.48-8.38 (m, 5H), 8.30 (s, OH), 8.25 (t, J=5.8 Hz, 1H), 8.15 (dt, J=3.4, 1.6 Hz, 2H), 7.67 (d, J=6.9 Hz, 1H), 7.37-7.24 (m, 5H), 5.12-5.01 (m, 2H), 4.03 (q, J=7.1 Hz, 1H), 3.91 (dd, J=21.2, 6.1 Hz, 4H), 2.49 (s, OH), 2.35-2.22 (m, 2H), 1.95 (ddt, J=15.0, 9.0, 5.9 Hz, 1H), 1.87-1.79 (m, 1H).

### Example 23. Synthesis of Compound 16

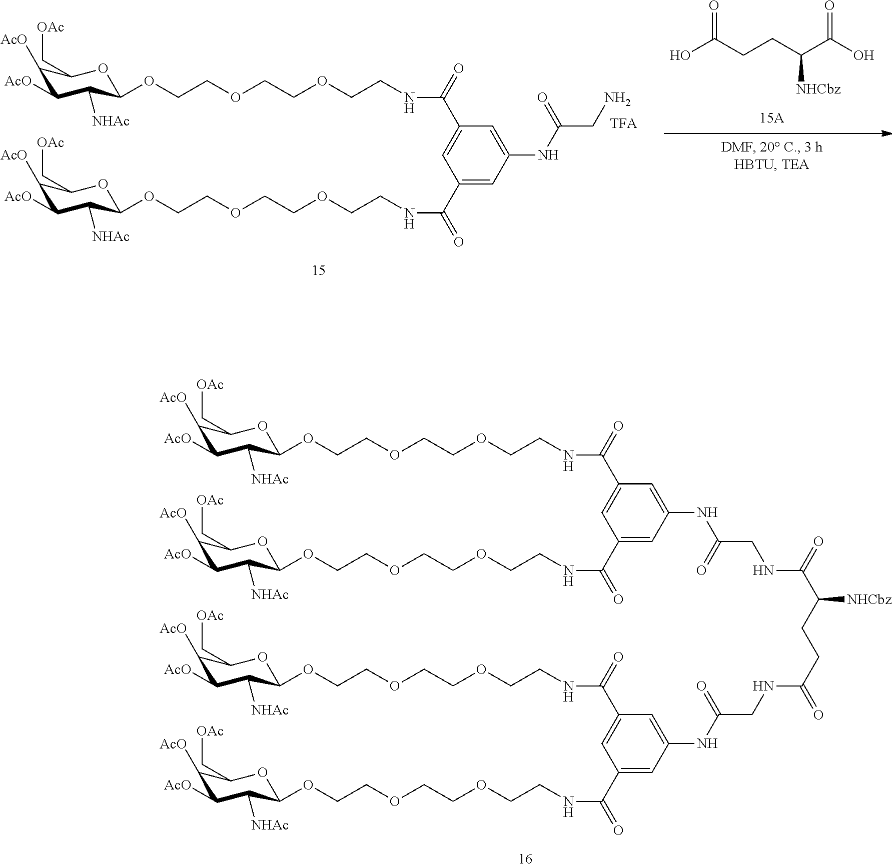

To a solution of compound 15 (40 g, 31.82 mmol) in DMF (400 mL), compound 15A (4.0 g, 14.32 mmol) and HBTU (14.5 g, 38.18 mmol) were added. The reaction mixture was cooled to 10° C. to 15° C. TEA (10.6 mL, 76.36 mmol) was added while maintaining internal temperature at 10° C. to 15° C. The reaction mixture allowed to warm to 20° C. to 25° C. and stir for not less than 3 hours until completion of reaction as confirmed by UPLC. He reaction mixture was quenched by addition of water (400 mL) and Ethyl acetate (400 mL). The aqueous layer was separated and extracted with DCM (3×400 mL). The DCM layers were pooled, washed with water (5×200 mL), dried over Na₂SO₄, filtered and evaporated to complete dryness. Compound 16 (31.7 g, 87% yield) was obtained as light-yellow foam solid. m/z 2563.9 [M+H]⁺

### Example 24. Synthesis of Compound 16

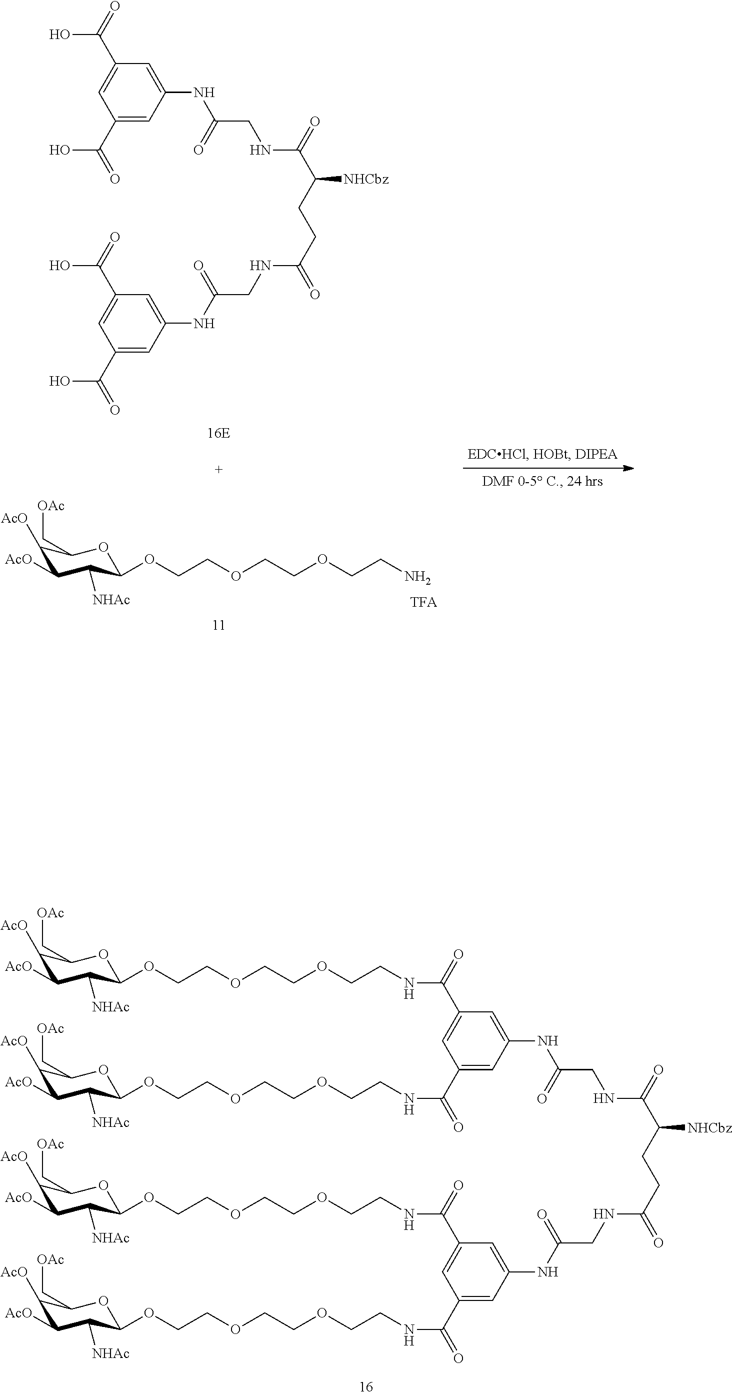

To a solution of compound 11 (1.0 g, 1.74 mmol) in anhydrous DMF (9 mL), compound 16E (0.21 g, 0.29 mmol)), EDC (333 mg, 1.74 mmol), HOBt (265 mg, 1.74 mmol) were sequentially added and the mixture cooled to 0° C. to 5° C. DIPEA (450 mg, 3.48 mmol) was added maintaining the internal temperature at 0° C. to 5° C. and allowed to stir at the same temperature for not less than 24 hours until completion of reaction as confirmed by UPLC. Reaction mixture diluted with water (11 mL), extracted with methylene chloride (50 mL). The methylene chloride layer was washed with water (2×5 mL), dried over Na₂SO₄ and evaporated to dryness. The residue was purified by column (2 to 15% MeOH/DCM) to give pure compound 16 (360 mg, 49.5% yield) as foamy solid.

### Example 25. Synthesis of Compound 16

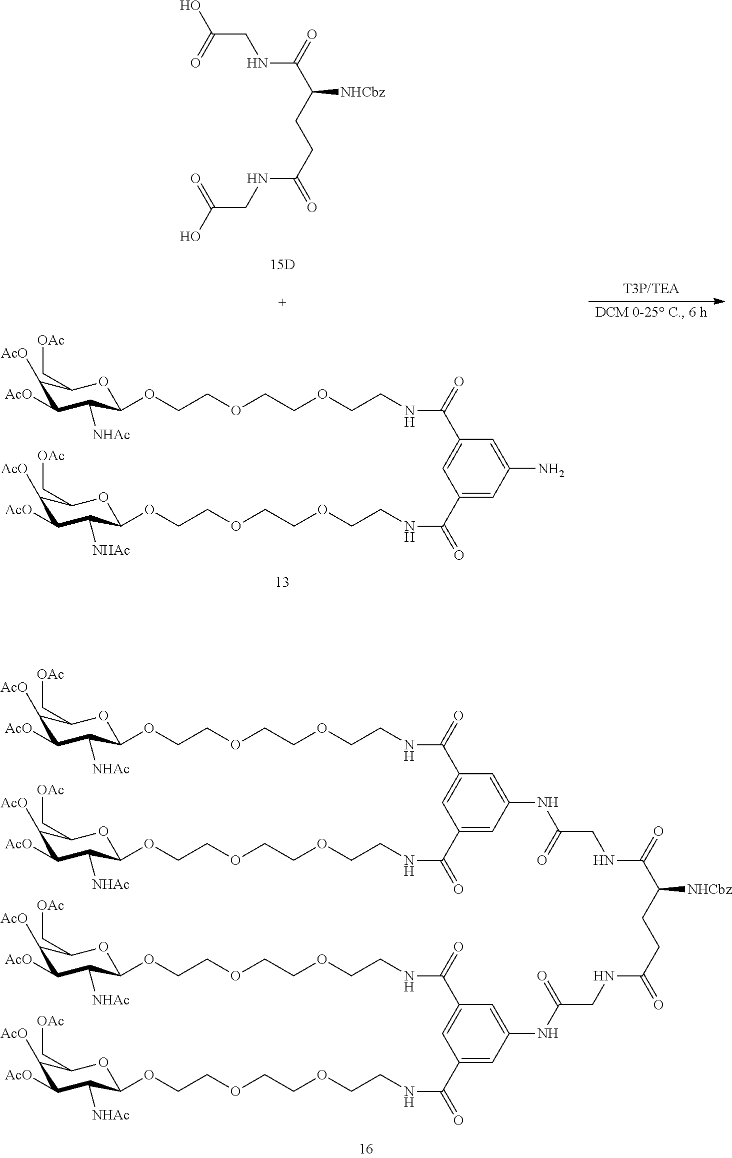

A solution of compound 15D (1.6 g, 4.08 mmol) and compound 13 (10 g, 9.07 mmol) in DCM (100 mL) was cooled to 0° C. to 10° C. TEA (1.84 g, 18.14 mmol) and T3P (10.34 mL, 16.3 mmol, 50% solution in ethyl acetate) were charged sequentially with agitation at 0-10° C. The reaction mixture was agitated at 25° C. to 35° C. for not less than 6 hours until completion of reaction as confirmed by UPLC. The reaction was quenched by addition of water (200 mL). The aqueous layer was separated and extracted with DCM (50 mL). The DCM layers were pooled and washed sequentially with saturated aq.NaHCO₃ (200 mL), 1.0 N HCl (200 mL), 10% aq. NaCl solution (200 mL), then dried over Na₂SO₄ (25 g) and evaporated to about 20 g. MTBE (50 mL) was added and evaporated to complete dryness and further dried in vacuum at 45° C. Compound 16 (10.3 g, 89% yield) was obtained as a pale yellow solid.

### Example 26. Synthesis of Compound 17

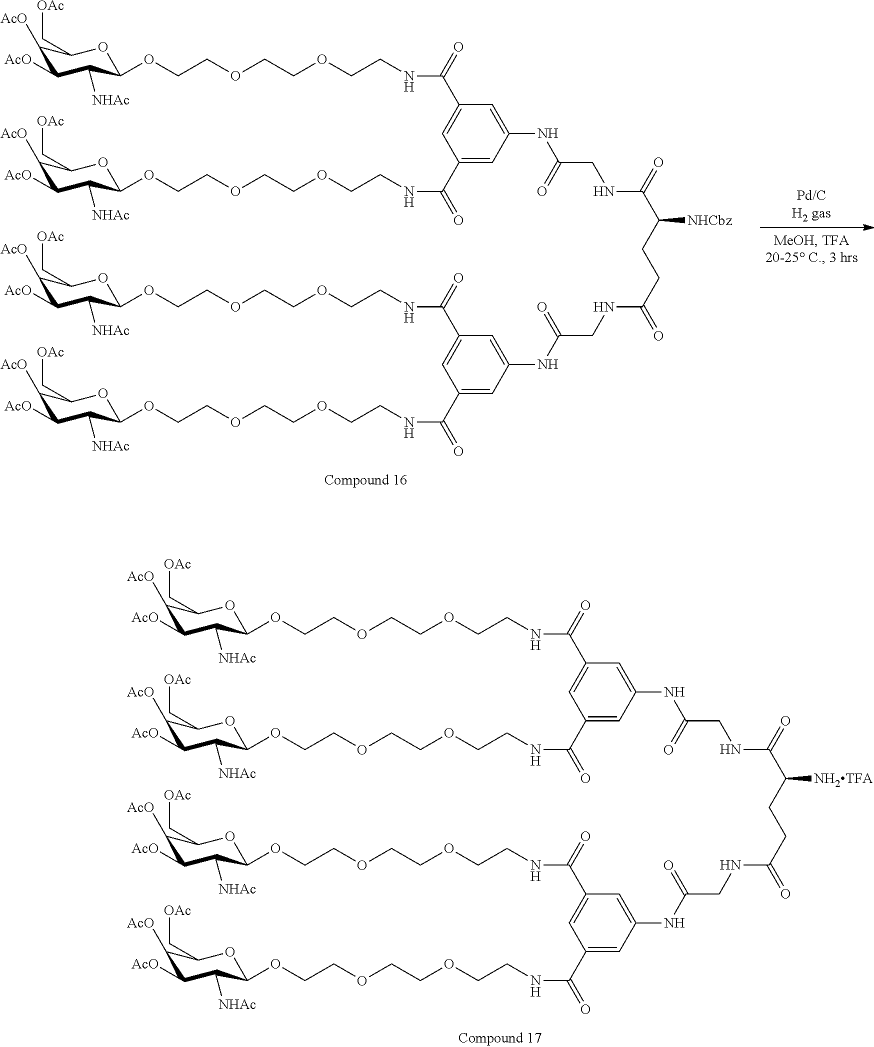

A solution of compound 16 (2.7 g, 1.05 mmol) in MeOH (27 mL) was degassed and purged with N₂, twice. 10 wt % Pd/C (0.27 g) and TFA (156 mg, 1.37 mmol) were charged and mixture was purged with H₂. The reaction mixture was allowed to agitate under H₂ atmosphere for not less than 3 hours until completion of reaction as confirmed by UPLC. The mixture was degassed and purged with N₂, filtered through a pad of celite. The filtrate was evaporated, residue taken in methylene chloride (25 mL), evaporated to complete dryness to give compound 17 (2.4 g, 90% yield) as grey foamy solid. m/z 2428.9 [M+H]⁺. ¹H NMR (600 MHz, DMSO-d₆) δ 8.54 (q, J=5.2 Hz, 1H), 8.21 (d, J=5.3 Hz, 1H), 8.14 (t, J=1.8 Hz, 1H), 7.95 (d, J=8.7 Hz, 1H), 7.80 (d, J=9.2 Hz, 1H), 5.19 (d, J=3.4 Hz, 1H), 4.95 (dd, J=11.2, 3.4 Hz, 1H), 4.52 (d, J=8.5 Hz, 1H), 4.07-3.97 (m, 3H), 3.94-3.82 (m, 2H), 3.76 (m, 1H), 3.59-3.47 (m, 8H), 3.44 (m, 3H), 2.38 (t, J=7.8 Hz, 1H), 2.08 (s, 3H), 1.98 (s, 3H), 1.87 (s, 3H), 1.75 (s, 3H).

### Example 27. Synthesis of Compound 18

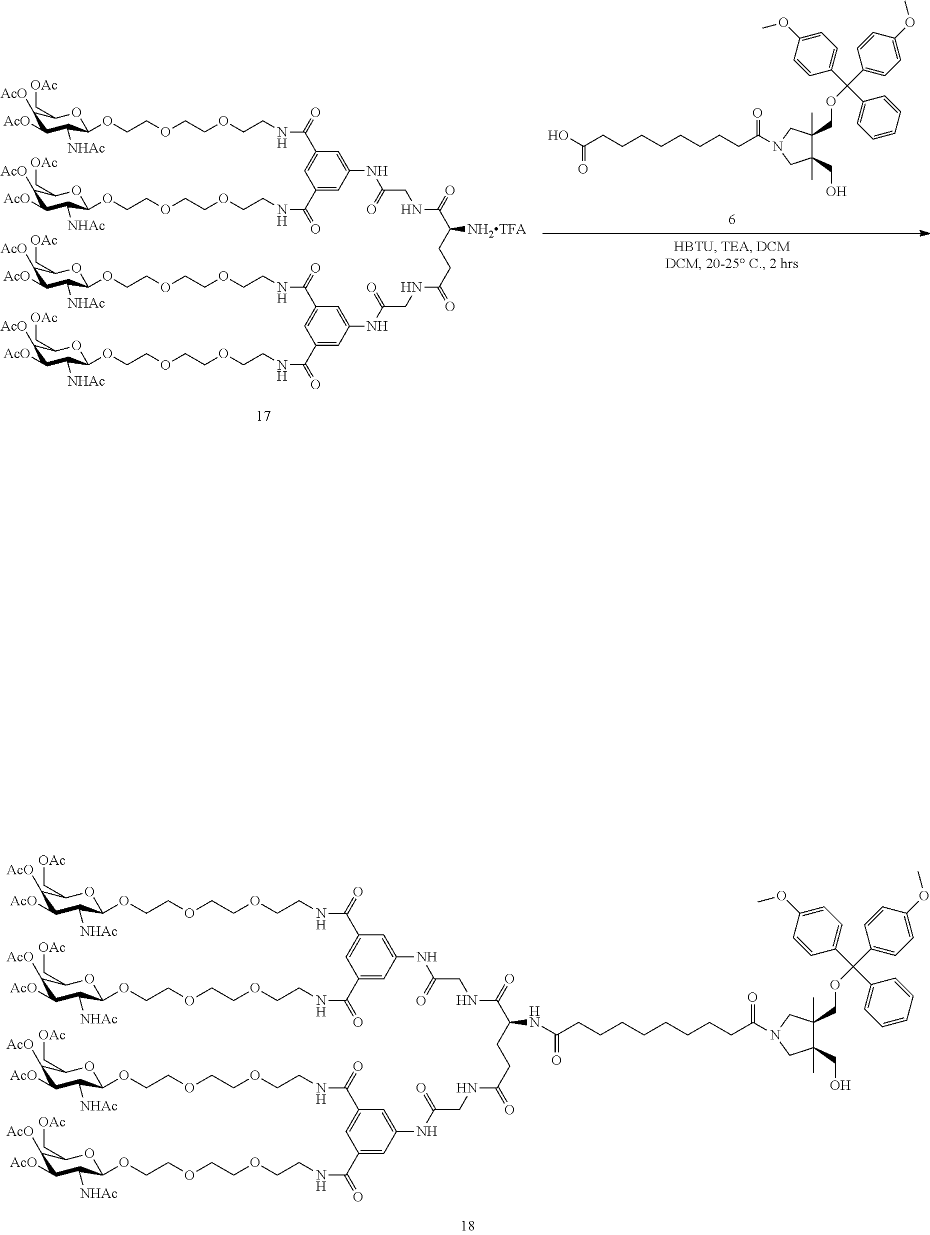

To a solution of compound 17 (1.0 g, 0.39 mmol) in DCM (25 mL), compound 6 (0.29 g, 0.44 mmol), HBTU (186 mg, 0.49 mmol), were added and mixture cooled to 15° C. to 25° C. DIPEA (151 mg, 1.17 mmol) was added maintaining the internal temperature at 15° C. to 25° C. and then allowed to stir at 20° C. to 25° C. for not less than 2.5 hours until completion of reaction as confirmed by UPLC. The reaction mixture diluted with DCM (5 mL) washed with water (10 mL), aq. NaHCO₃ solution (3×8 mL), brine (10 mL), dried over Na₂SO₄ and evaporated to dryness. The residue was purified by column (2 to 18% MeOH/DCM) to give pure compound 18 (860 mg, 72.5% yield) as off-white foamy solid. m/z (z=2) 1378.5 [M−DMTr+2H]²⁺.

### Example 28. Synthesis of Compound 18

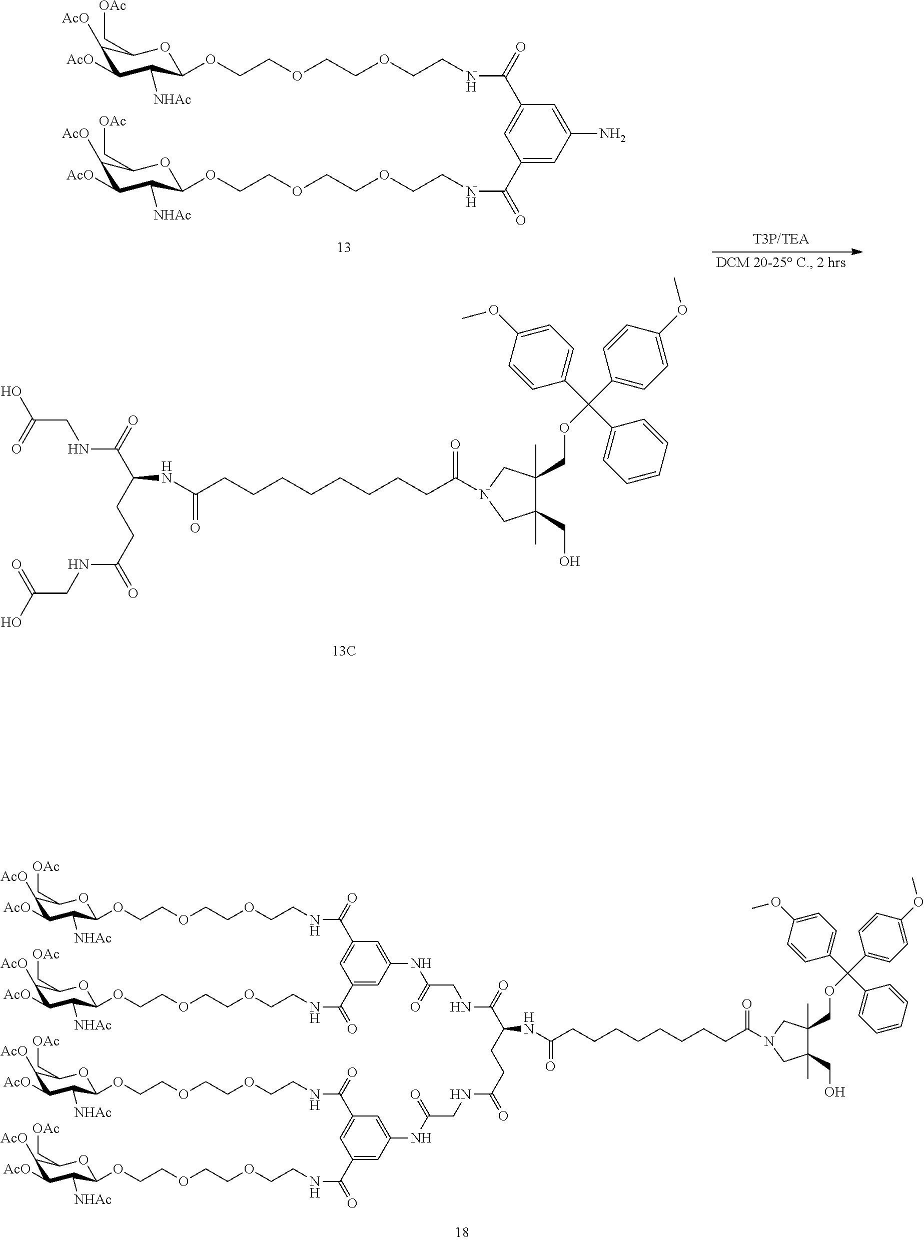

A solution of compound 13C (39.8 g, 40.2 mmol) and compound 13 (93 g, 84.39 mmol) in THF (800 mL) was cooled to 0 to 10° C. TEA (0.5 mL, 3.6 mmol) and T3P (20.3 g, 200.9 mmol, 50% solution in ethyl acetate) were charged sequentially with agitation at 0-10° C. The reaction mixture was agitated at 25 to 35° C. for not less than 18 hours until completion of reaction as confirmed by UPLC. The reaction mixture was quenched by addition of saturated aq.NaHCO₃ (800 mL) (10 mL) and 2-MeTHF (800 mL). The aqueous layer was separated. The organic layer was washed sequentially with 5% NaH2PO4 (800 mL), 10% aq. NaCl solution (800 mL), then dried over Na₂SO₄ and evaporated. Solvent swapped to MTBE (400 mL), allowed to stir for 3-4 h and evaporated to complete dryness and further dried in vacuum at 45° C. Crude compound 18 (120 g) was obtained as a solid. Crude material is purified by column chromatography to >97% purity and taken to next step. m/z (z=2) 1378.5 [M-DMTr+2H]²⁺.

### Example 29. Synthesis of Compound 19

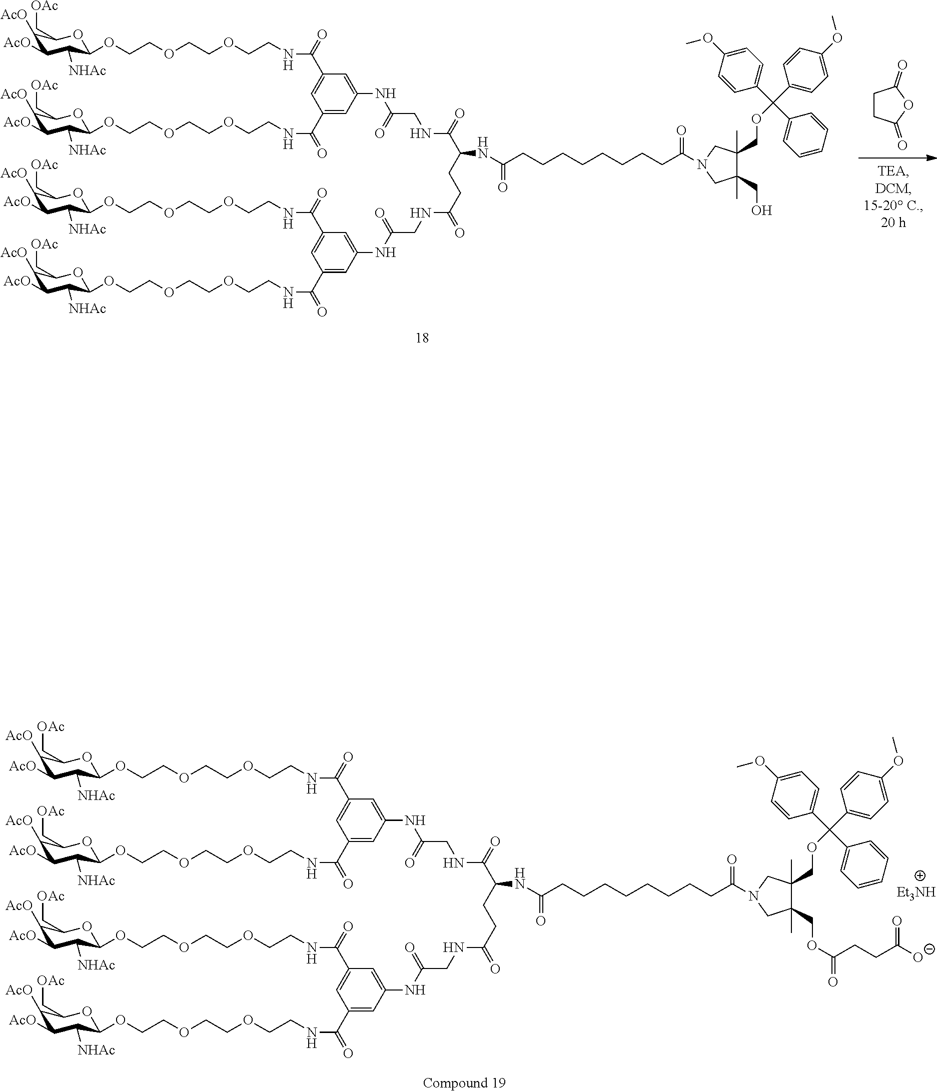

To a solution of compound 18 (1.1 Kg, 163.6 mmol) in DCM (1.1 L), TEA (126 g, 1260 mmol) was charged slowly charged keeping the temperature at 25° C. Compound 18A (126 g, 1260 mmol) was then charged in portions maintaining the temperature at 25° C. The resulting mixture was agitated at 40 to 45° C. for not less than 72 hours until completion of reaction as confirmed by UPLC. The reaction mixture was cooled to 20° C. to 25° C. and washed with aq. NaHCO₃ solution (2×5 L), dried over Na₂SO₄, filtered and evaporated to complete dryness. Compound 19 was obtained as off-white solid (1.0 Kg). For free acid: m/z (z=2) 1428.5 [M−DMTr+2H]²⁺.

### Example 30. Synthesis of the Crystalline Potassium Salt of the Compound of Formula 13CC

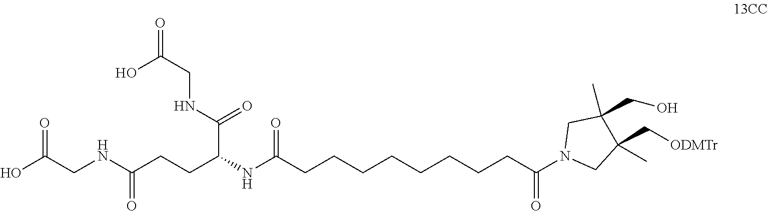

5.0 g (1.0 equiv) of Compound 13BB, and 25 mL (5 V) of MeOH were charged to a reactor (100 mL). After dissolution, the contents were adjusted to 0-5° C. 653 mg (2.2 equiv) of KOH was dissolved with 25 mL (5 V) of MeOH in another reactor. KOH in MeOH was slowly charged to the contents, and the reaction mixture was slowly adjusted to 40° C. The reaction mixture was agitated until the reaction was complete. After concentration to minimum volume, 10 V of CPME was charged and the contents were agitated at 50-60° C. During the agitation, a pale yellow slurry formed. The slurry was concentrated to minimum volume under reduced pressure. After charging 10 V of heptane, the slurry was agitated at 50-60° C. for 1 hour and slowly adjusted to 0-5° C. After agitation for 1 hour, the contents were filtered via filter paper and the wet cake was washed with 2 V of heptane. 5.2 g of product was obtained as a off-white solid.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for preparing a compound of formula 1: 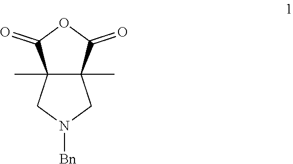 [SMILES: C[C@@]12CN(Cc3ccccc3)C[C@]1(C)C(=O)OC2=O]
comprising reacting a compound of formula 1-1: 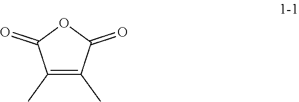
with a compound of formula 1-2: 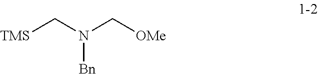 [SMILES: COCN(Cc1ccccc1)C[Si](C)(C)C]
at a temperature of 40° C. or greater.

2. The method of claim 1 wherein the compound of formula 1-1 is reacted with the compound of formula 1-2 in a solvent that comprises tetrahydrofuran.

3. The method of claim 1 or 2 wherein the compound of formula 1-1 is reacted with the compound of formula 1-2 in a solvent that comprises tetrahydrofuran at a temperature of 60° C. or greater.

4. A method for preparing a crystalline form of compound 3: 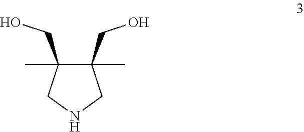
comprising converting a compound of formula 1: 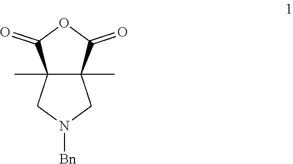 [SMILES: C[C@@]12CN(Cc3ccccc3)C[C@]1(C)C(=O)OC2=O]
to the crystalline form of compound 3 without using column chromatography during the conversion.

5. A crystalline form of compound 3:

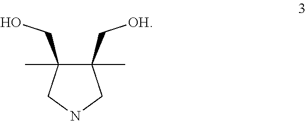

6. A method for preparing a compound of formula 9: 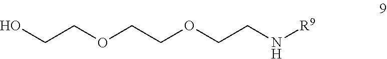
wherein R⁹ is an optionally substituted benzyloxycarbonyl group, comprising converting a compound of formula 8: 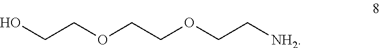
or a salt thereof to the compound of formula 9.

7. The method of claim 6 wherein R⁹ is benzyloxycarbonyl or nitrobenzyloxycarbonyl.

8. The method of claim 6 wherein the compound of formula 8 is converted to the compound of formula 9 by treating the compound of formula 8 with benzyloxycarbonyl chloride in a suitable solvent in the presence of a suitable base.

9. A method for preparing a compound of formula 10: 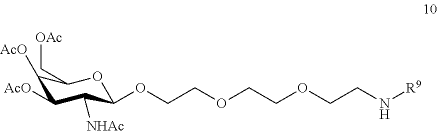
wherein R⁹ is an optionally substituted benzyloxycarbonyl group, comprising converting a corresponding compound of formula 9: 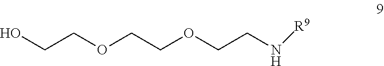
to the compound of formula 10.

10. The method of claim 9 wherein the compound of formula 9 is converted to the compound of formula 10 by treatment with a compound of formula 7: 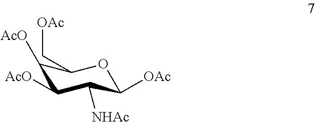
in the presence of a suitable catalyst and a suitable solvent.

11. The method of claim 10 wherein the catalyst is Sc(OTf)₃ and wherein the suitable solvent comprises dichloroethane.

12. A method for preparing a compound of formula 10: 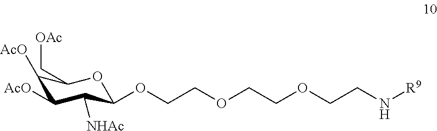
wherein R⁹ is an optionally substituted benzyloxycarbonyl group, comprising converting a compound of formula 8: 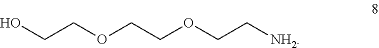
or a salt thereof to a corresponding compound of formula 9; 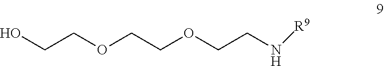
and subsequently converting the corresponding compound of formula 9 to the compound of formula 10, without purifying the compound of formula 9 by chromatography.

13. A method for preparing a salt of formula 11: 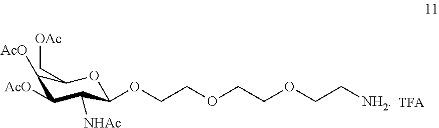
comprising treating a compound of formula 10: 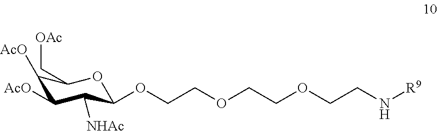
wherein R⁹ is an optionally substituted benzyloxycarbonyl group, with hydrogen and trifluoroacetic acid in the presence of a suitable catalyst and in the presence of a suitable solvent.

14. The method of claim 13 wherein the suitable catalyst comprises palladium on carbon and wherein the suitable solvent comprises tetrahydrofuran.

15. A method for preparing a compound of formula 15D: 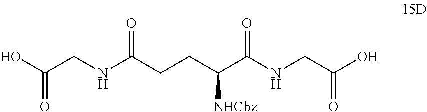 [SMILES: O=C(O)CNC(=O)CC[C@H](NC(=O)OCc1ccccc1)C(=O)NCC(=O)O]
or a salt thereof, comprising converting a compound of formula 15C: 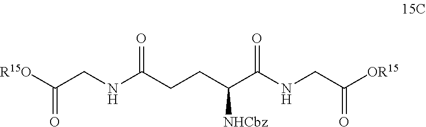
wherein each R¹⁵ is a (C₁-C₆)alkyl, to the compound of formula 15D or the salt thereof.

16. A method for preparing a compound of formula 15C: 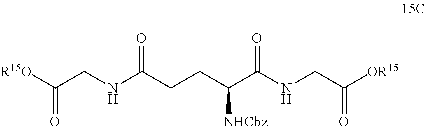
wherein each R¹⁵ is a (C₁-C₆)alkyl, comprising reacting a compound of formula 15A: 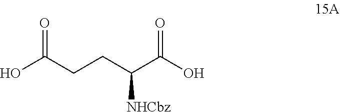 [SMILES: O=C(O)CC[C@H](NC(=O)OCc1ccccc1)C(=O)O]
or a salt thereof, with a corresponding compound of formula 15B: 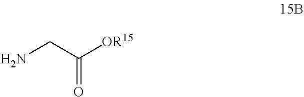
or a salt thereof, to provide the compound of formula 15C.

17. A method for preparing a compound of formula 13A: 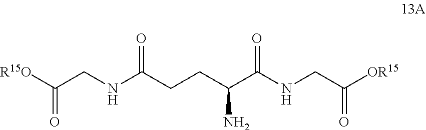
wherein each R¹⁵ is a (C₁-C₆)alkyl, comprising converting a corresponding compound of formula 15C: 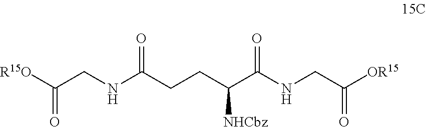
wherein each R¹⁵ is a (C₁-C₆)alkyl, to the compound of formula 13A.

18. A method for preparing a compound of formula 13B: 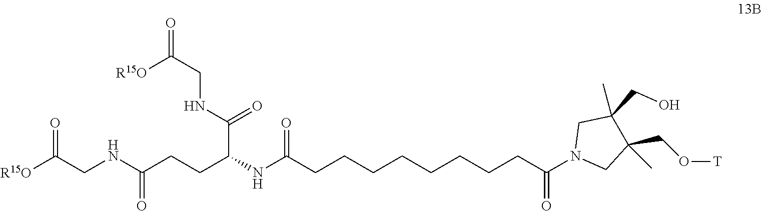
wherein each R¹⁵ is a (C₁-C₆)alkyl and T is an optionally substituted triphenylmethyl group, comprising converting a corresponding compound of formula 13A: 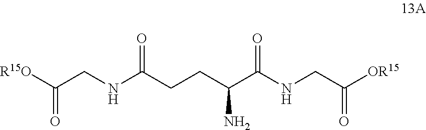
to the compound of formula 13B.

19. The method of claim 18 wherein the compound of formula 13A is converted to the compound of formula 13B, by treating the compound of formula 13A with a corresponding compound of formula 6: 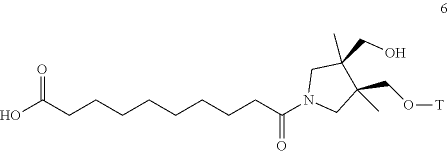
or a salt thereof, under suitable amide forming conditions.

20. A method for preparing a compound of formula 13CC: 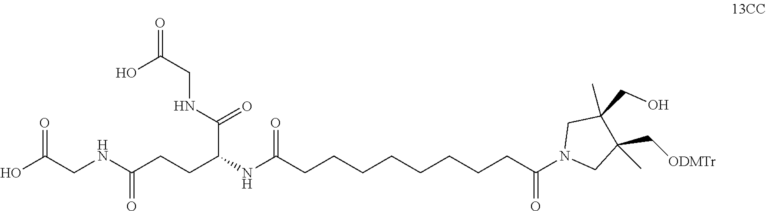
comprising converting a compound of formula 13BB: 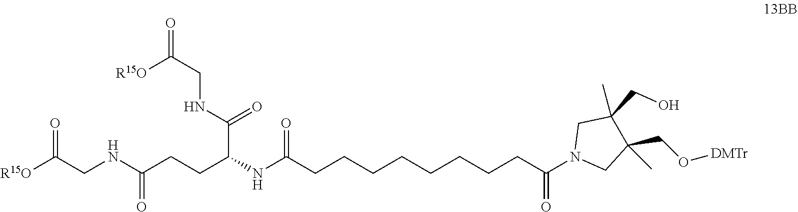
wherein each R¹⁵ is a (C₁-C₆)alkyl, to the compound of formula 13CC.

21. The method of claim 20 wherein the compound of formula 13BB is converted to the compound of formula 13CC by treatment with lithium hydroxide in a suitable solvent.

22. A method for preparing a potassium salt of a compound of formula 13CC: 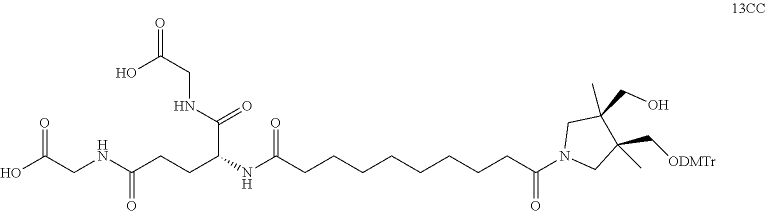
comprising treating a compound of formula 13CC or a salt thereof with potassium carbonate in a suitable solvent to provide the potassium salt of the compound of formula 13CC.

23. A method for preparing a compound of formula 11B: 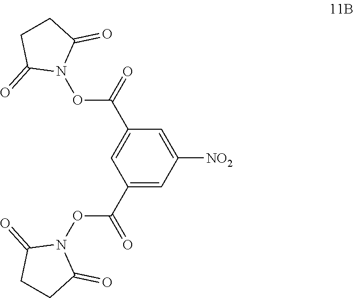 [SMILES: O=C(ON1C(=O)CCC1=O)c1cc(C(=O)ON2C(=O)CCC2=O)cc([N+](=O)[O-])c1]
comprising converting a compound of formula 11A: 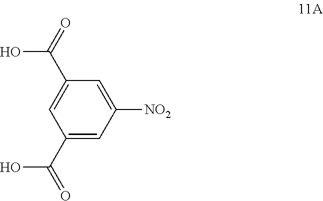 [SMILES: O=C(O)c1cc(C(=O)O)cc([N+](=O)[O-])c1]
or a salt thereof, to the compound of formula 11B.

24. The method of claim 23 wherein the compound of formula 11A is converted to the compound of formula 11B by treating the compound of formula 11A or the salt thereof with ethyl-3-(3-dimethylaminopropyl)carbodiimide in a suitable solvent.

25. A method for preparing a compound of formula 12: 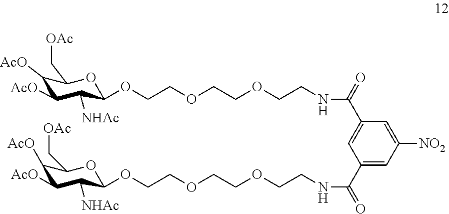
comprising converting a compound of formula 11B: 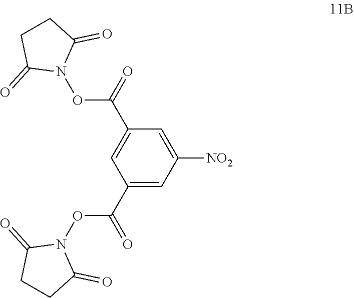 [SMILES: O=C(ON1C(=O)CCC1=O)c1cc(C(=O)ON2C(=O)CCC2=O)cc([N+](=O)[O-])c1]
to the compound of formula 12.

26. The method of claim 25, wherein the compound of formula 11B is converted to the compound of formula 12 by treating the compound of formula 11B with a compound of formula 11: 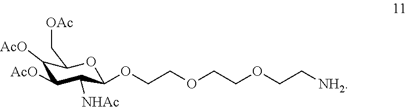
or a salt thereof, in the presence of a suitable base and a suitable solvent.

27. A method for preparing a compound of formula 13: 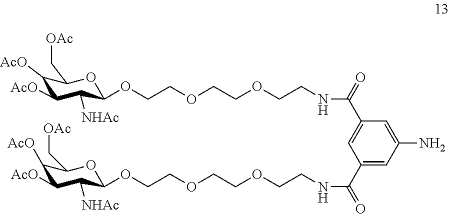
or a salt thereof, comprising reducing a compound of formula 12: 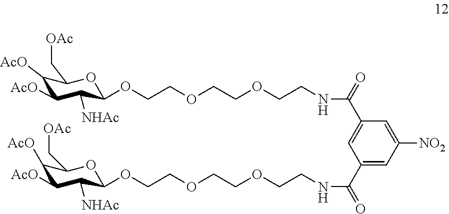
to provide the compound of formula 13 or the salt thereof.

28. The method of claim 27, wherein the compound of formula 13 or a salt thereof, is a trifluoroacetic acid salt of the following formula:

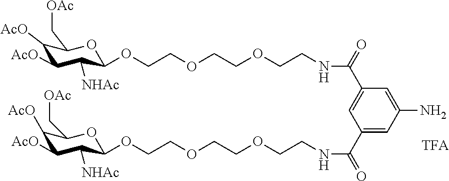

29. A method for preparing a compound of formula 14: 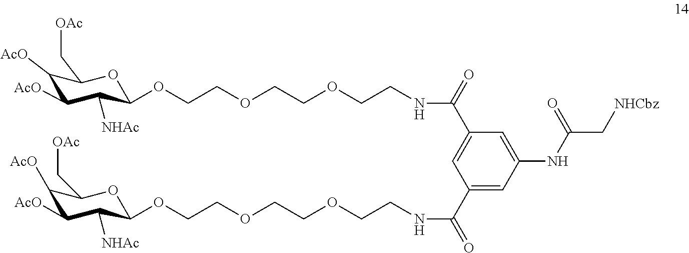
comprising converting a compound of formula 13: 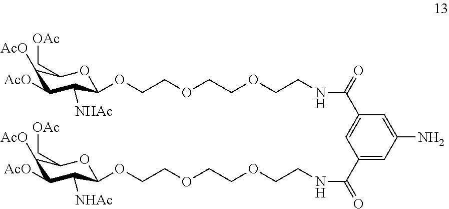
or a salt thereof to the compound of formula 14.

30. The method of claim 29, wherein the compound of formula 13 is converted to the compound of formula 14, by treating the compound of formula 13 with a compound of formula: 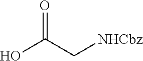 [SMILES: O=C(O)CNC(=O)OCc1ccccc1]
or a salt thereof, under suitable coupling conditions.

31. A method for preparing a compound of formula 16: 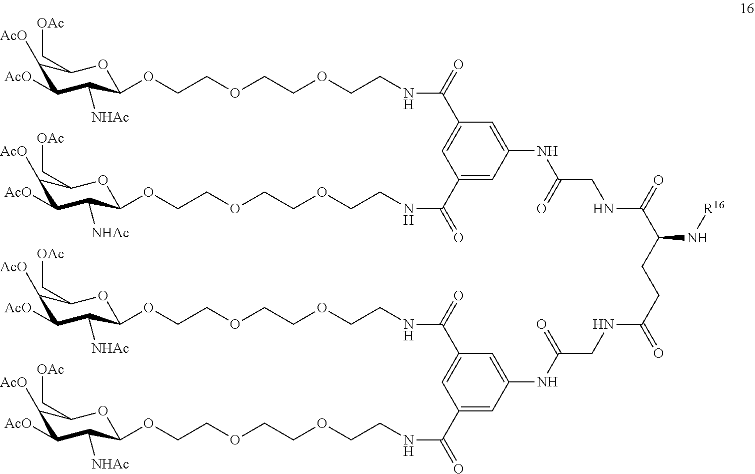
wherein R¹⁶ is an amine protecting group, comprising converting a compound of formula 13: 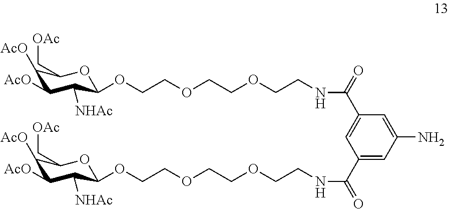
or a salt thereof, to the compound of formula 16.

32. The method of claim 31, wherein the compound of formula 13 or the salt thereof is converted to the compound of formula 16, by treating the compound of formula 13 with a compound of formula 15DD: 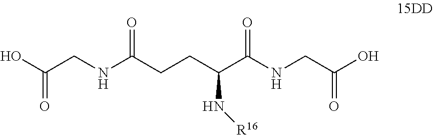
wherein R¹⁶ is an amine protecting group, or a salt thereof, under suitable coupling conditions.

33. The method of claim 31, wherein a trifluoroacetic acid salt of a compound of formula 13: 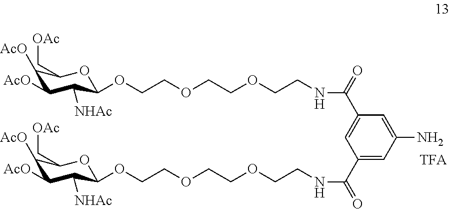
is treated with a compound of formula 15D: 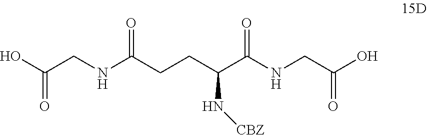
under suitable coupling conditions to provide a compound of formula 16: 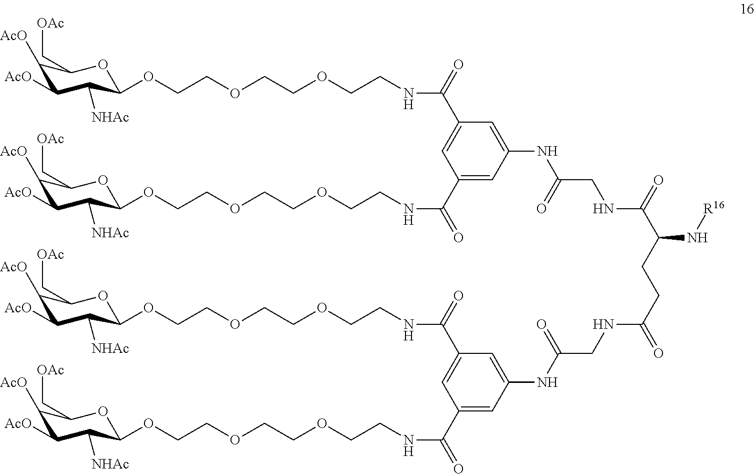
wherein R¹⁶ is benzyloxycarbonyl.

34. The method of any one of claims 31-33, wherein the compound of formula 13 is treated with the compound of formula 15D or 15DD in the presence of propanephosphonic acid anhydride and a solvent comprising dichloromethane to provide the compound of formula 16.

35. A method for preparing a compound of formula 18: 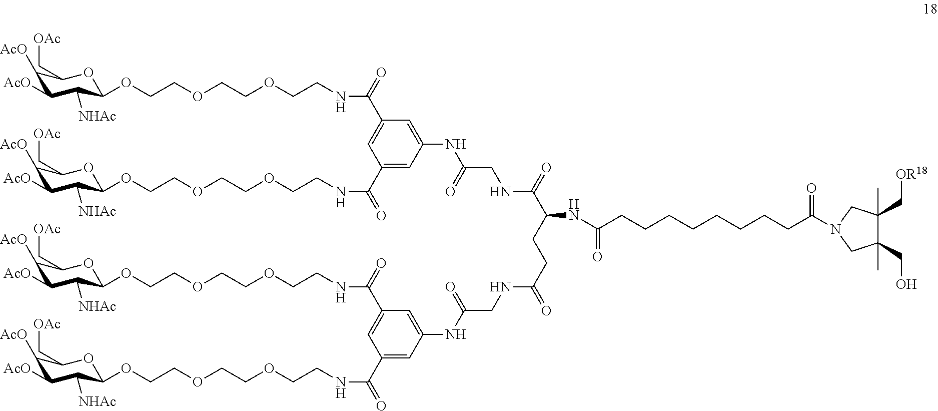
wherein R¹⁸ is a suitable protecting group, comprising converting a compound of formula 13: 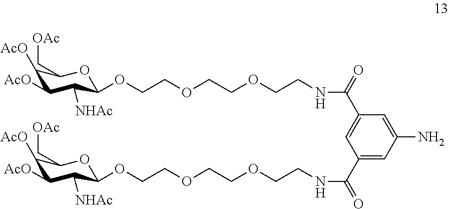
or a salt thereof, to the compound of formula 18.

36. The method of claim 35, wherein the compound of formula 13 or the salt thereof is converted to the compound of formula 18, by treating the compound of formula 13 with a compound of formula 13CCC: 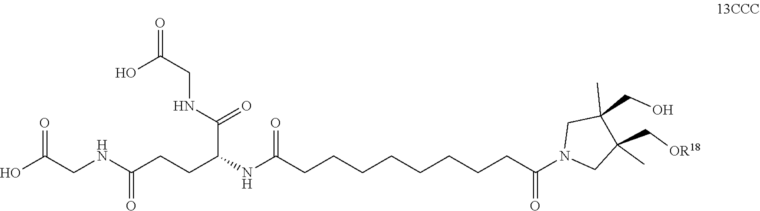
wherein R¹⁸ is a suitable protecting group, or a salt thereof, under suitable coupling conditions.

37. The method of claim 35, wherein a trifluoroacetic acid salt of a compound of formula 13: 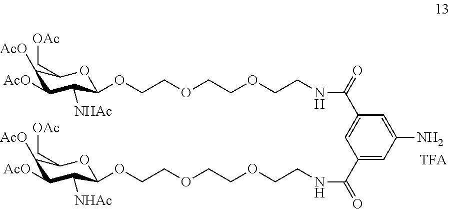
is treated with a compound of formula 13CCC, wherein R¹⁸ is 4,4-dimethoxytriphenylmethyl under suitable coupling conditions to provide a compound of formula 18: 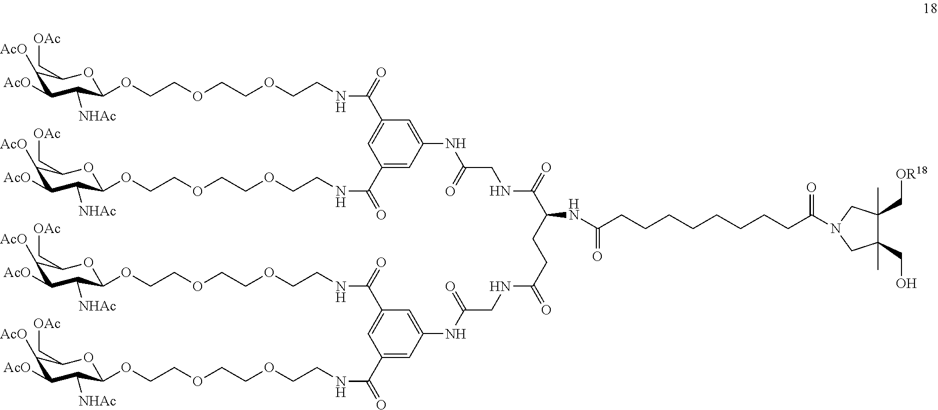
wherein R¹⁸ is 4,4-dimethoxytriphenylmethyl.

38. The method of any one of claims 35-37, wherein the compound of formula 13 is treated with the compound of formula 13CCC in the presence of propanephosphonic acid anhydride and a solvent comprising dichloromethane to provide the compound of formula 18.

39. A method for preparing a compound of formula 16-2: 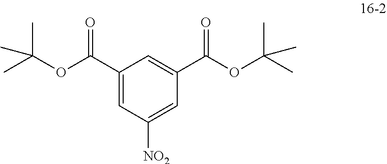 [SMILES: CC(C)(C)OC(=O)c1cc(C(=O)OC(C)(C)C)cc([N+](=O)[O-])c1]
comprising converting a compound of formula 16-1: 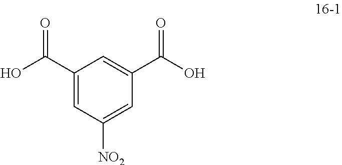 [SMILES: O=C(O)c1cc(C(=O)O)cc([N+](=O)[O-])c1]
or a salt thereof, to the compound of formula 16-2.

40. A method for preparing a compound of formula 16-3: 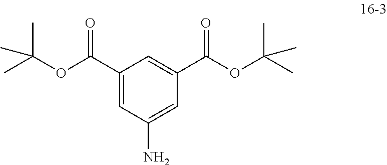 [SMILES: CC(C)(C)OC(=O)c1cc(N)cc(C(=O)OC(C)(C)C)c1]
comprising converting a compound of formula 16-2: 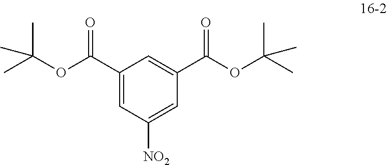 [SMILES: CC(C)(C)OC(=O)c1cc(C(=O)OC(C)(C)C)cc([N+](=O)[O-])c1]
to the compound of formula 16-3.

41. A method for preparing a compound of formula 16-4: 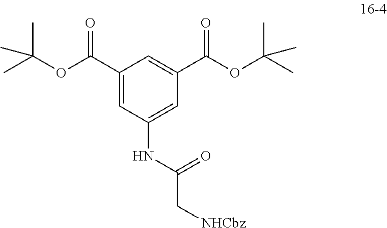 [SMILES: CC(C)(C)OC(=O)c1cc(NC(=O)CNC(=O)OCc2ccccc2)cc(C(=O)OC(C)(C)C)c1]
comprising converting a compound of formula 16-3: 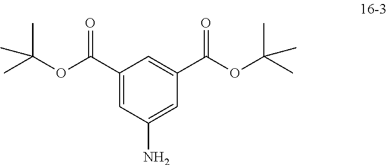 [SMILES: CC(C)(C)OC(=O)c1cc(N)cc(C(=O)OC(C)(C)C)c1]
to the compound of formula 16-4.

42. A method for preparing a compound of formula 16-5: 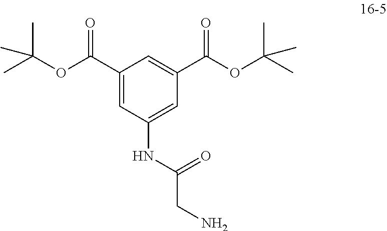 [SMILES: CC(C)(C)OC(=O)c1cc(NC(=O)CN)cc(C(=O)OC(C)(C)C)c1]
or a salt thereof, comprising converting a compound of formula 16-4: 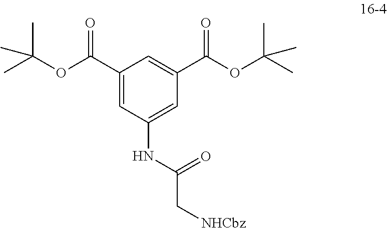 [SMILES: CC(C)(C)OC(=O)c1cc(NC(=O)CNC(=O)OCc2ccccc2)cc(C(=O)OC(C)(C)C)c1]
to the compound of formula 16-5.

43. A method for preparing a compound of formula 16D: 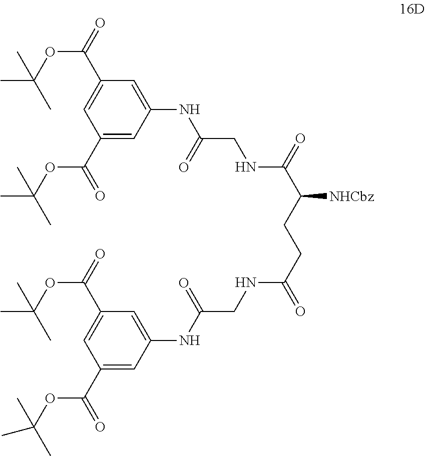 [SMILES: CC(C)(C)OC(=O)c1cc(NC(=O)CNC(=O)CC[C@H](NC(=O)OCc2ccccc2)C(=O)NCC(=O)Nc2cc(C(=O)OC(C)(C)C)cc(C(=O)OC(C)(C)C)c2)cc(C(=O)OC(C)(C)C)c1]
or a salt thereof, comprising converting a compound of formula 16-5: 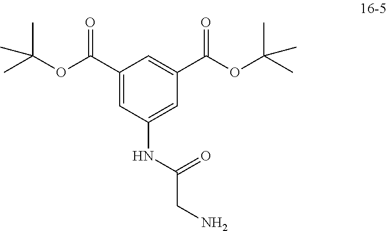 [SMILES: CC(C)(C)OC(=O)c1cc(NC(=O)CN)cc(C(=O)OC(C)(C)C)c1]
to the compound of formula 16D.

44. A method for preparing a compound of formula 16E: 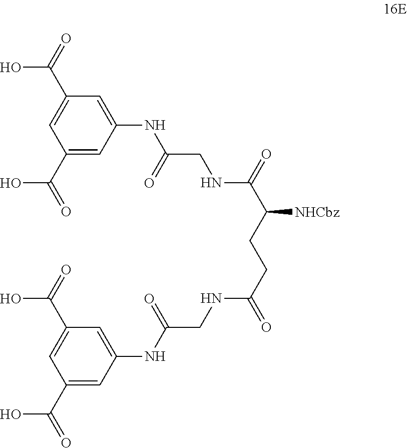 [SMILES: O=C(CC[C@H](NC(=O)OCc1ccccc1)C(=O)NCC(=O)Nc1cc(C(=O)O)cc(C(=O)O)c1)NCC(=O)Nc1cc(C(=O)O)cc(C(=O)O)c1]
or a salt thereof, comprising converting a compound of formula 16D: 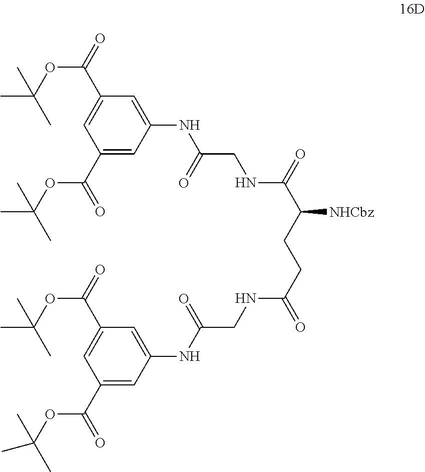 [SMILES: CC(C)(C)OC(=O)c1cc(NC(=O)CNC(=O)CC[C@H](NC(=O)OCc2ccccc2)C(=O)NCC(=O)Nc2cc(C(=O)OC(C)(C)C)cc(C(=O)OC(C)(C)C)c2)cc(C(=O)OC(C)(C)C)c1]
or a salt thereof, to the compound of formula 16D.

45. A method for preparing a compound of formula 16: 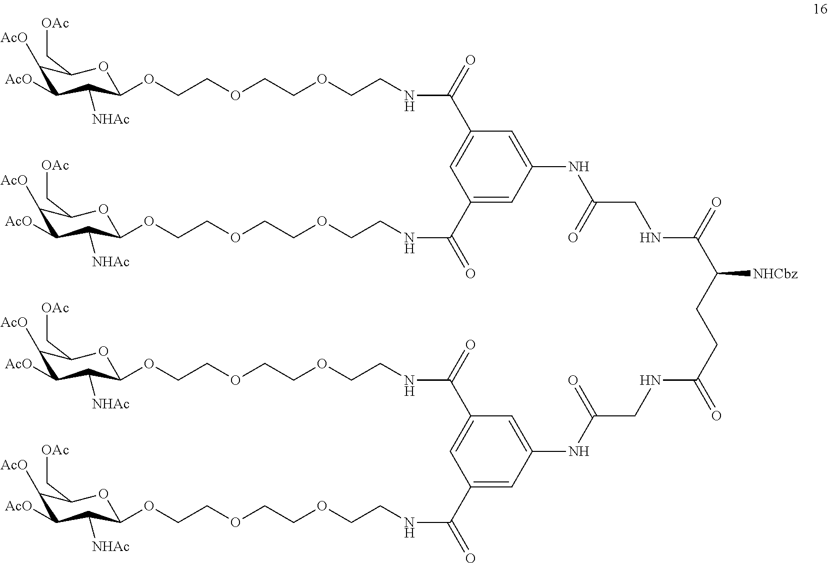
or a salt thereof, comprising converting a compound of formula 16E: 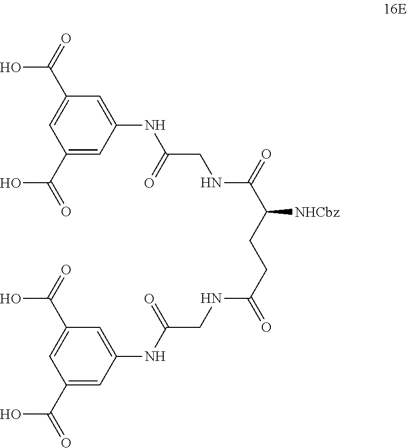 [SMILES: O=C(CC[C@H](NC(=O)OCc1ccccc1)C(=O)NCC(=O)Nc1cc(C(=O)O)cc(C(=O)O)c1)NCC(=O)Nc1cc(C(=O)O)cc(C(=O)O)c1]
or a salt thereof, to the compound of formula 16.

46. The method of claim 45, wherein the compound of formula 16E or the salt thereof is converted to the compound of formula 16 or the salt thereof, by reacting the compound of formula 16E or the salt thereof with a compound of formula 11: 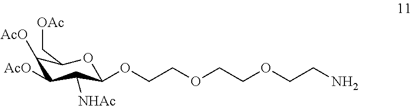
or a salt thereof, under suitable coupling conditions.

47. A compound selected from the group consisting of: 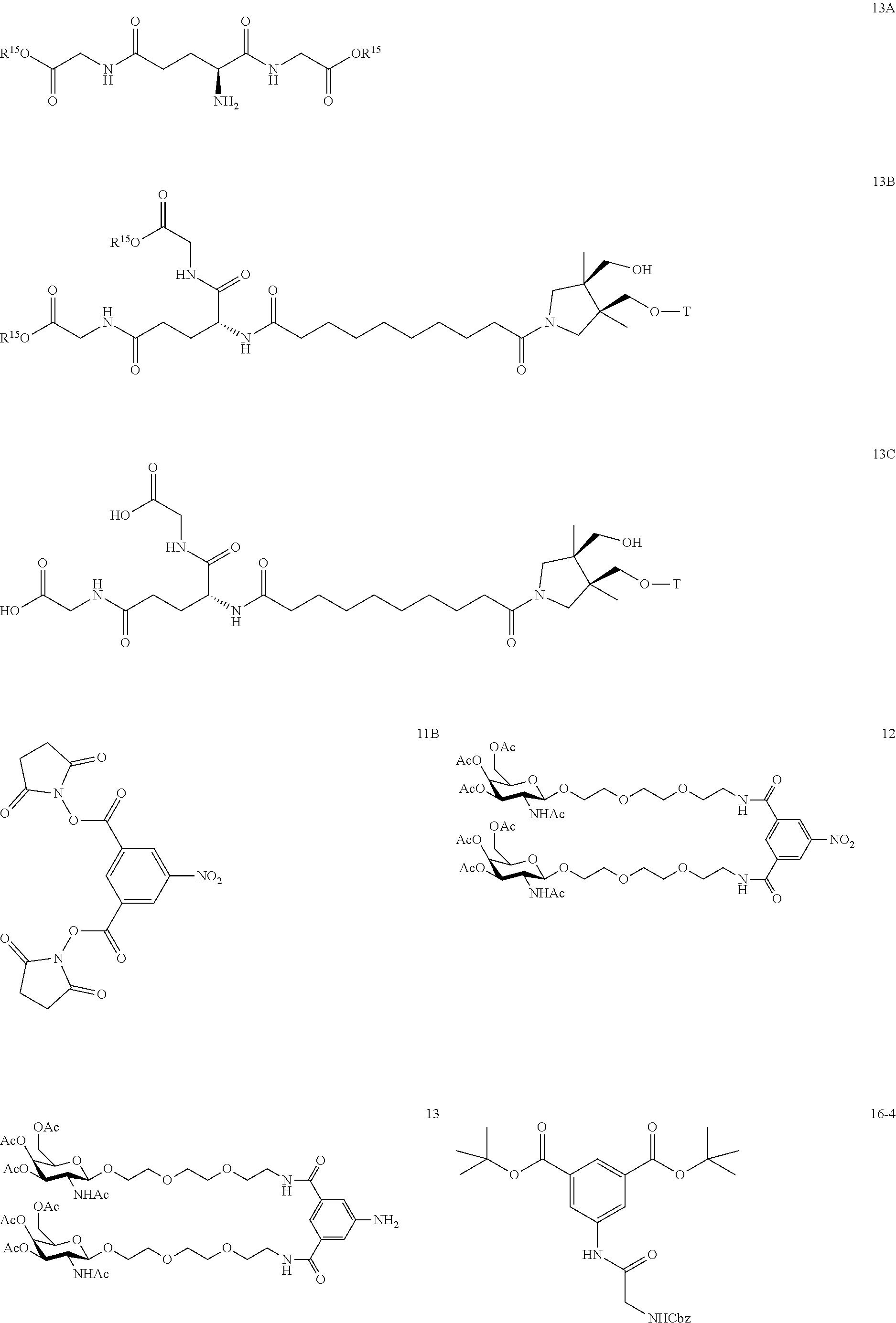 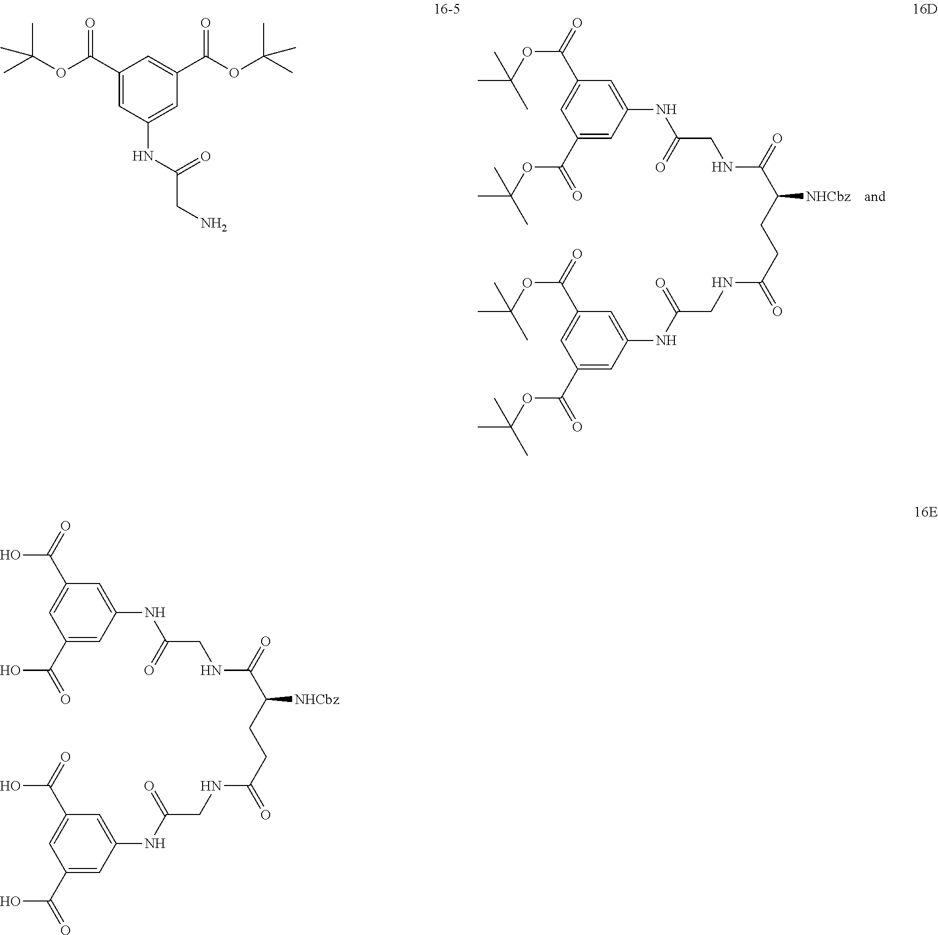 [SMILES: CC(C)(C)OC(=O)c1cc(NC(=O)CN)cc(C(=O)OC(C)(C)C)c1 ; CC(C)(C)OC(=O)c1cc(NC(=O)CNC(=O)CC[C@H](NC(=O)OCc2ccccc2)C(=O)NCC(=O)Nc2cc(C(=O)OC(C)(C)C)cc(C(=O)OC(C)(C)C)c2)cc(C(=O)OC(C)(C)C)c1 ; "and" ; O=C(CC[C@H](NC(=O)OCc1ccccc1)C(=O)NCC(=O)Nc1cc(C(=O)O)cc(C(=O)O)c1)NCC(=O)Nc1cc(C(=O)O)cc(C(=O)O)c1]
or a salt thereof, wherein each R¹⁵ is (C₁-C₆)alkyl and each T is an optionally substituted triphenylmethyl group.

48. A compound selected from the group consisting of: 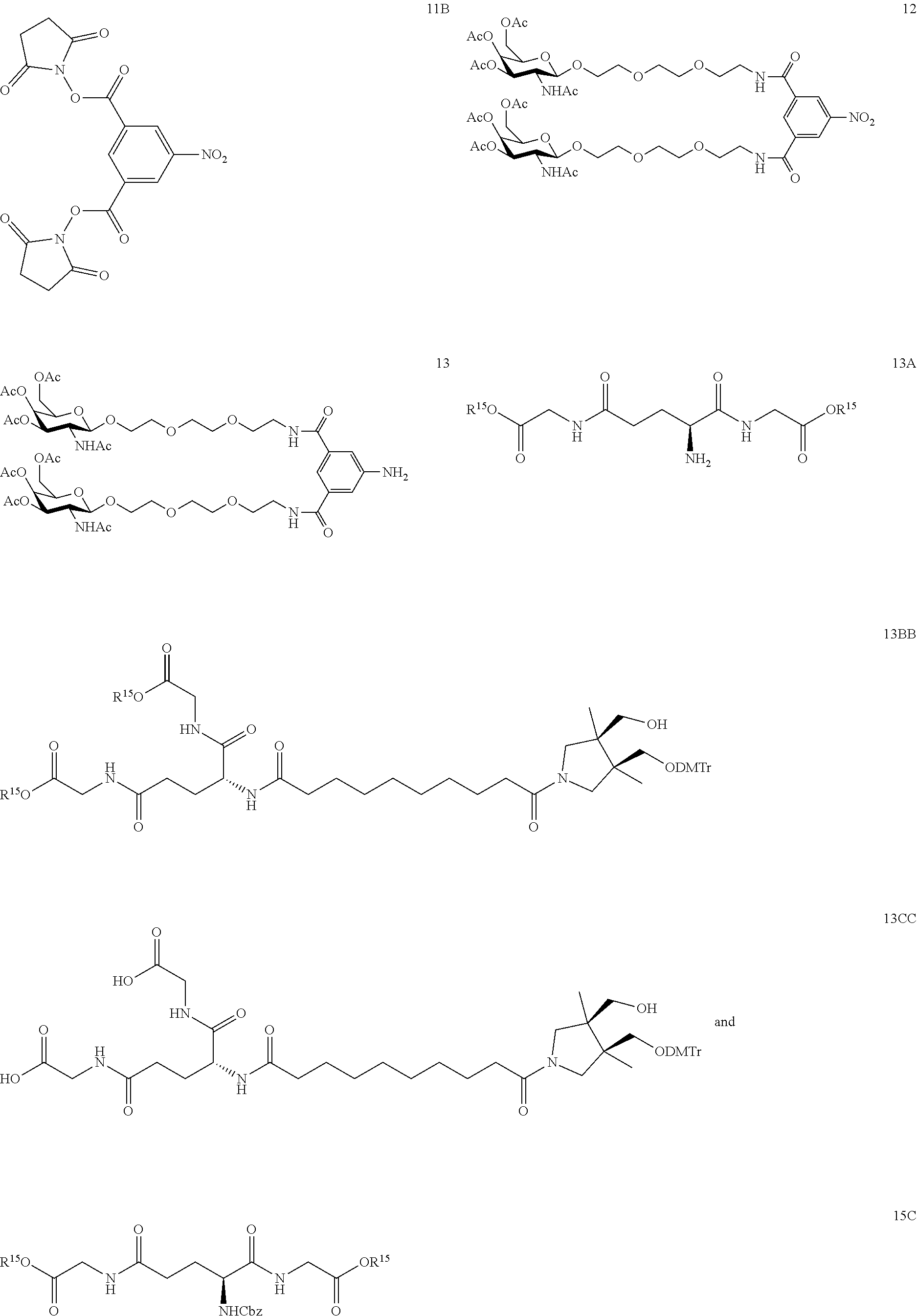
or a salt thereof, wherein each R¹⁵ is (C₁-C₆)alkyl.

49. The salt:

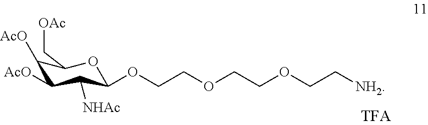

50. The method of any one of claims 35-38, further comprising converting the compound of formula 18 to a compound of formula 19: 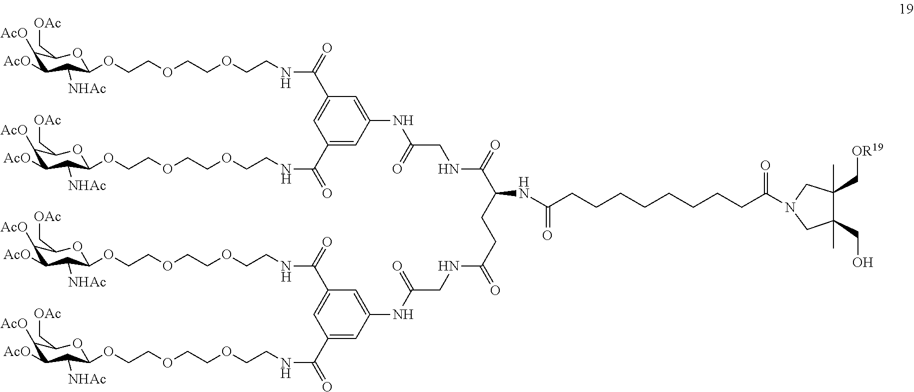
wherein R¹⁹ is a group that comprises an siRNA that is suitable for treating HBV and/or HDV.

51. The method of claim 50, wherein the compound of formula 19 is a compound of formula 20: 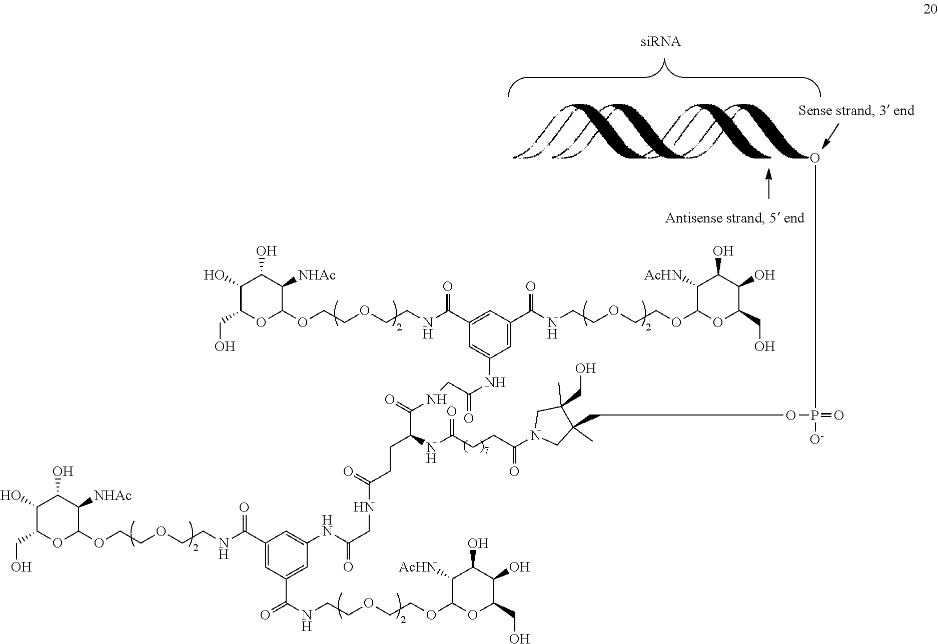
wherein the siRNA is suitable for treating HBV and/or HDV.

52. A method for treating HBV and/or HDV infection in a human subject comprising administering to the human subject, a therapeutically effective amount of compound of formula 19 or formula 20 that is prepared as described in claim 50 or 51, and a second therapeutic agent that is useful for treating HBV and/or HDV.

53. The method of claim 52 wherein the second therapeutic agent is an HBV capsid formation inhibitor or an HBV RNA destabilizer.

54. The method of claim 53 wherein the HBV RNA destabilizer is an HBV surface antigen inhibitor.

55. The method of any one of claims 52-54 wherein the compound of formula 19 or formula 20 and the second therapeutic agent are administered separately.
